(19) United States
(12) Patent Application Publication
(10) Pub. No.: US 2006/0178307 A1
(43) Pub. Date: Aug. 10, 2006

(54) Modulation of NMDA receptor currents via orexin receptor and/or CRF receptor

(75) Inventors: Selena Bartlett, Berkeley, CA (US); Antonello Bonci, San Francisco, CA (US); Stephanie Borgland, San Francisco, CA (US); Howard Fields, Berkeley, CA (US); Sharif Taha, Berkeley, CA (US)

Correspondence Address: QUINE INTELLECTUAL PROPERTY LAW GROUP, P.C., P O BOX 458, ALAMEDA, CA, 94501, US

(73) Assignee: The Regents of the University of California

(21) Appl. No.: 11/343,259

(22) Filed: Jan. 25, 2006

Related U.S. Application Data
(60) Provisional application No. 60/647,748, filed on Jan. 26, 2005.

(51) Int. Cl. A61K 38/22; A61K 31/495; A61K 31/4745; A61K 31/4706; A61K 31/17

(52) U.S. Cl. 514/12; 514/255.01; 514/300; 514/313; 514/585; 514/595

(57) ABSTRACT

This invention pertains to the discoveries that orexin and/or CRF increase NMDAR (N-methyl-D-aspartate receptor)-mediated currents at excitatory synapses onto a subset of dopamine cells in the ventral tegmental area (VTA) in the mammalian brain. The orexin effect can be blocked by an orexin receptor type 1 (OXR1). The CRF effect can be blocked by a CRF receptor 2 (CRF-R2) antagonist or by an inhibitor of the CRF-binding protein (CRF-BP). Methods are provided that exploit these discoveries to modulate NMDAR-mediated currents in vivo and in vitro and to screen for modulators (upregulators or downregulators) of NMDA-mediated currents. In vivo methods include the use of modulators of the orexin and CRF pathways to of mitigate a symptom of substance abuse. The invention also provides methods and compositions for co-administration of modulators that act via the orexin and CRF pathways.

### CROSS-REFERENCE TO RELATED APPLICATIONS

This application claims the benefit of U.S. Provisional Application No. 60/647,748, filed Jan. 26, 2005, which is hereby incorporated by reference in its entirety.

### STATEMENT AS TO RIGHTS TO INVENTIONS MADE UNDER FEDERALLY SPONSORED RESEARCH AND DEVELOPMENT

This invention was made with government support under grant no. NIH 1RO1DA15096-01. The Government may have certain rights in the invention.

### FIELD OF THE INVENTION

This invention pertains to the field of neurobiology. In particular this invention pertains to the discovery that the orexin receptor and/or the CRF receptor can potentiate activity at an NMDA receptor and to methods of screening for agents that modulate such potentiation.

### BACKGROUND OF THE INVENTION

Orexins (hypocretins) are two alternatively spliced neuropeptides that are synthesized solely in the lateral hypothalamus (LHA) and appear to be critically involved in arousal, feeding and motivation, and behaviors that are linked to corticolimbic dopamine function (de Lecea et al., 1998; Sakurai et al., 1998). Several lines of evidence suggest that orexin modulates dopaminergic neurotransmission. Previous morphological analysis has shown that terminals of LHA orexin neurons are apposed to dendrites and somata of dopaminergic neurons of ventral tegmental area (VTA; Fadel and Deutch, 2002). Secondly, the dopamine receptor antagonist, haloperidol, blocks hyperlocomotion and stereotypy induced by intracerebroventricular orexin (Nakamura et al., 2000). Finally, orexin increased firing rate of VTA neurons and in some cases, caused burst firing (Korotkova et al., 2003), which is associated with amplified dopamine release (Overton and Clark, 1997).

The actions of orexin A and B are mediated by two G protein-coupled receptors termed orexin receptor type 1 (OXR1) and type 2 (OXR2); OXR1 shows higher affinity for orexin A, while OXR2 shows equal affinity for the two ligands (Sakurai et al., 1998).

Corticotrophin-releasing factor (CRF), a 41 amino acid peptide, plays an obligatory role in the activation of the hypothalamic-pituitary-adrenal axis and the subsequent release of glucocorticoids in response to stressful events (Koob and Heinrichs (1999) Brain Res. 848: 141-152; Kaufman et al. (2000) Biol. Psychiatry 48: 778-790; Behan et al. (1995) Nature 378: 284-287; Sarnyai et al. (2001) Pharmacol. Rev. 53: 209-243). In addition, extra-hypothalamic CRF mediates many behavioral responses to stress (Koob and Heinrichs (1999) Brain Res. 848: 141-152). Altered CRF levels are seen in a number of psychiatric and neurological disorders, such as depression and Alzheimer's disease (Kaufman et al. (2000) Biol. Psychiatry 48: 778-790; Behan et al. (1995) Nature 378: 284-287 (1995)). CRF is elevated in animal models of withdrawal from drugs of abuse and plays a key role in stress-induced relapse to drug taking (Sarnyai et al. (2001) Pharmacol. Rev. 53: 209-243). The cellular effects of CRF are mediated via two receptors (CRF-R1 and CRF-R2) (Dautzenberg and Hauger (2002) Trends Pharmacol. Sci. 23, 71-77); CRF also binds to a binding protein (CRF-BP), which is thought to inactivate ‘free’ CRF (Kemp et al. (1998) Peptides 19: 1119-1128). It has been suggested that CRF-BP inhibitors, which elevate ‘free’ CRF levels, may provide potential treatments for disorders where CRF levels are depressed, such as Alzheimer's disease and Parkinson's disease (Behan et al. (1995) Nature 378: 284-287). It is notable that many of these disorders involving elevated CRF levels are also thought to involve elevated dopamine levels and that CRF increases dopamine release in both limbic and cortical projection areas (Koob and Heinrichs (1999) Brain Res. 848: 141-152; Kaufman et al. (2000) Biol. Psychiatry 48: 778-790; Dunn and Berridge (1987) Pharmacol. Biochem. Behav. 27: 685-691). How CRF modulates dopaminergic activity, however, is unclear.

Dopamine neurons in the ventral tegmental area (VTA) are under important regulatory control from excitatory glutamatergic projections from a number of brain regions, such as the prefrontal cortex and amygdala, and modulation of these synapses is involved in both short- and long-term changes in dopaminergic activity (Bonci and Malenka (1999) J Neurosci. 19: 3723-3730; Overton et al. (1999) Neuroreport. 10: 221-226; Ungless et al. (2001) Nature 411: 584-587). In particular, N-methyl-D-aspartate receptors (NMDARs) play a key role in regulating burst firing and the induction of long-term synaptic potentiation in these neurons (8. Bonci and Malenka (1999) J Neurosci. 19: 3723-3730; Overton et al. (1999) Neuroreport. 10: 221-226; Ungless et al. (2001) Nature 411: 584-587; Overton and Clark (1997) Brain Res. Brain Res. Rev. 25: 312-334). Interestingly, stress-induced activation of the dopamine system requires NMDAR activity (Morrow et al. (1993) Eur. J. Pharmacol. 238: 255-262), and repeated stress induces increases in NMDAR and α-amino-3-hydroxy-5-methyl-isoxazolepropionic acid receptor (AMPAR) subunits in the VTA (Fitzgerald et al. (1996) J Neurosci. 16: 274-282). Although CRF has been shown to modulate neuronal excitability in a number of brain regions (Aldenhoff et al. (1983) Science 221: 875-877; Valentino et al. (1983) Brain Res. 270: 363-367), increase field potentials and prime population-spike long-term potentiation (LTP) in the hippocampus (Wang et al. (1998) Eur. J. Neurosci. 10: 3428-3437; Blank et al. (2002) J. Neurosci. 22: 3788-3794), its role in the modulation of excitatory synaptic transmission is poorly understood.

### SUMMARY OF THE INVENTION

The invention provides a method of modulating a N-methyl-D-aspartate receptor (NMDAR)-mediated current that entails administering to a mammal, an orexin receptor agonist or antagonist in a concentration sufficient to alter said NMDAR-mediated current. Also provided is a method of mitigating a symptom of substance abuse in a mammal that entails administering to the mammal, an orexin receptor antagonist in a concentration sufficient to reduce or prevent a symptom of substance abuse. The invention also includes a method of modulating a N-methyl-D-aspartate receptor (NMDAR)-mediated current in a dopaminergic neuron by modulating binding between orexin and the orexin receptor type 1 (OXR1). In another embodiment, the invention provides a method of modulating the activity of corticotrophin-releasing factor (CRF) on a dopaminergic neuron by modulating binding between orexin and the orexin receptor type 1 (OXR1). When an orexin receptor agonist or antagonist is administered to a mammal, preferably the mammal is one that is not being treated for an eating disorder.

In embodiments wherein the method comprises administering an orexin receptor antagonist to downregulate the NMDAR-mediated current, any suitable antagonist can be employed. Exemplary antagonists include tetrahydroisoquinolines, aroyl piperazine derivatives, 1-(2-methylbenzoxazol-6-yl)-3-[1,5]naphthyridin-4-yl urea hydrochloride (SB-334867-A), N-(6,8-difluoro-2-methyl-4-quinolinyl)-N′-[4-(dimethylamino) phenyl]urea (SN-408124), phenyl urea derivatives, and phenyl thiourea derivatives.

In embodiments wherein the method comprises administering an orexin receptor agonist to upregulate the NMDAR-mediated current, any suitable agonist can be employed. Exemplary agonists include orexin A, orexin B, and [Ala11,D-Leu15]-orexin B.

When an orexin receptor agonist or antagonist is administered to reduce or prevent a symptom of substance abuse, the substance of abuse can be, but is not limited to, an opioid, a psychostimulant, a sedative-hypnotic drug, a cannabinoid, an empathogen, a dissociative drug, alcohol, and nicotine. In particular embodiments, the substance of abuse is morphine, a barbiturate, cocaine, an amphetamine, alcohol, or nicotine. Exemplary symptoms of substance abuse that can be addressed using the methods of the invention include reward, incentive salience, craving, preference, seeking, and/or intake (self-administration) of said substance of abuse; relapse; and a symptom of withdrawal.

In a variation of the above-described methods, a CRF receptor agonist or antagonist can be administered in conjunction with an orexin receptor agonist or antagonist. More specifically, (1) an orexin receptor antagonist can be administered in conjunction with a CRF receptor antagonist; (2) an orexin receptor antagonist can be administered in conjunction with a CRF receptor agonist; (3) an orexin receptor agonist can be administered in conjunction with a CRF receptor antagonist, and (4) an orexin receptor agonist can be administered in conjunction with a CRF receptor agonist. The orexin receptor agonist or antagonist can be administered simultaneously, or sequentially, with the CRF receptor agonist or antagonist.

Another aspect of the invention is composition including an orexin receptor agonist or antagonist combined with a CRF receptor agonist or antagonist. Such compositions can include any of the specific combinations noted above, with respect to the coadministration method.

In particular embodiments of the above-described methods and compositions of the invention, the orexin receptor agonist or antagonist is selective for the orexin receptor type 1 (OXR1).

The invention also provides a method of screening for an agent that modulates orexin potentiation of N-methyl-D-aspartate receptor (NMDAR)-mediated currents. In one embodiment, the method entails: (1) contacting a cell with a test agent; and (2) detecting the expression or activity of an orexin receptor type 1 (OXR1); wherein an alteration of expression or activity of an OXR1 receptor as compared to a control indicates that said test agent is an agent that modulates orexin potentiation of NMDAR-mediated currents. In various embodiments, the cell employed in the screening method can, for example, be: a nerve cell, a cell in a neurological tissue, a cell in a brain slice preparation, and/or a nerve cell in culture.

Detection, in the screening method, can include detecting an electrophysiological signal from a nerve cell. In particular embodiments, an electrophysiological signal is detected from a dopamine neuron, and more particularly, one in a ventral tegmental area (VTA).

In certain embodiments, detection includes detecting an OXR1 receptor nucleic acid, preferably by nucleic acid hybridization. Examples of nucleic acid hybridization assays useful in the invention include a Northern blot, a Southern blot using DNA derived from a OXR1 receptor RNA, an array hybridization, an affinity chromatography, and an in situ hybridization.

Detection can include detecting an OXR1 receptor protein. In particular embodiments, detection is accomplished by binding a OXR1 receptor protein with a detectable label. Examples of protein-based assays useful in the invention include capillary electrophoresis, a Western blot, mass spectroscopy, ELISA, immunochromatography, and immunohistochemistry.

Preferably, the control employed in the screening methods includes a cell contacted with the test agent at a lower concentration or a cell that is not contacted with the test agent.

In another embodiment, the invention provides a method of screening for an agent that modulates the activity of orexin on a dopaminergic neuron, wherein the method entails: (1) contacting a test agent with an orexin and/or an orexin receptor type 1 (OXR1); and (2) detecting an increase or decrease in interaction between said orexin and said OXR1 receptor where an increase or decrease in said interaction, as compared to a control, indicates that said test agent modulates the activity of orexin on a dopaminergic neuron. In particular embodiments, the interaction is in vitro, e.g., in a cultured neural cell or a brain slice preparation.

In preferred embodiments of this screening method, detection is carried out by detecting specific binding of said test agent to one or more of said components. Examples of assays that can be employed in the screening method include a two-hybrid system and a gel-shift assay.

The preferred test agent for the screening methods of the invention is a small organic molecule.

Definitions

A “symptom of substance abuse” includes any symptom, i.e., any effect or behavior, arising from substance abuse. Thus, a symptom of substance abuse arises from the previous, and/or ongoing, use of a substance. Examples include, but are not limited to, elevated: reward, incentive salience, craving, preference, seeking, and/or intake (self-administration) of the substance, as compared to that in a normal population (i.e., one that is not using the substance in a maladaptive manner) and relapse, as well as any of the individual symptoms of substance dependence and/or addiction listed below.

“Substance dependence” includes a maladaptive pattern of substance use, leading to clinically significant impairment or distress, as manifested by three (or more) of the following symptoms, occurring at any time in the same 12-month period:

(1) Tolerance, as defined by either of the following: (a) a need for markedly increased amounts of the substance to achieve intoxication or desired effect, or (b) markedly diminished effect with continued use of the same amount of the substance;

(2) Withdrawal, as manifested by either of the following: (a) the characteristic withdrawal syndrome for the substance, or (b) the same (or closely related) substance is taken to relieve or avoid withdrawal symptoms;

(3) The substance is often taken in larger amounts or over a longer period than was intended;

(4) There is a persistent desire or unsuccessful efforts to cut down or control substance use;

(5) A great deal of time is spent in activities necessary to obtain the substance (e.g., visiting multiple doctors or driving long distances), use the substance (e.g., chain-smoking), or recover from its effects;

(6) Important social, occupational, or recreational activities are given up or reduced because of substance use; and

(7) The substance use is continued despite knowledge of having a persistent or recurrent physical or psychological problem that is likely to have been caused or exacerbated by the substance (e.g., current cocaine use despite recognition of cocaine-induced depression, or continued drinking despite recognition that an ulcer was made worse by alcohol consumption). (See American Psychiatric Association, Diagnostic Criteria for DSM-IV, Washington D.C., APA, 1994.)

“Substance addiction” includes a maladaptive pattern of substance use leading to clinically significant impairment or distress, as manifested by one (or more) of the following, occurring within a 12-month period:

(1) recurrent substance use resulting in a failure to fulfill major role obligations at work, school, or home (e.g., repeated absences or poor work performance related to substance use; substance-related absences, suspensions, or expulsions from school; neglect of children or household);

(2) recurrent substance use in situations in which it is physically hazardous (e.g., driving an automobile or operating a machine when impaired by substance use);

(3) recurrent substance-related legal problems (e.g., arrests for substance-related disorderly conduct); and

(4) continued substance use despite having persistent or recurrent social or interpersonal problems caused or exacerbated by the effects of the substance (e.g., arguments with spouse about consequences of intoxication, physical fights). (See American Psychiatric Association, Diagnostic Criteria for DSM-UV, Washington D.C., APA, 1994.)

As used with respect to substance abuse, the term “reward” refers to the tendency of a substance to cause pleasurable effects that induce a subject to alter their behavior to obtain more of the substance.

As used with respect to substance abuse, the term “incentive salience” refers to a particular form of motivation to consume a previously experienced substance that results from a hypersensitive neural state thought to be mediated by dopaminergic systems.

As used with respect to substance abuse, the term “craving” refers to the desire to experience the effects of a previously experienced substance or to ameliorate the negative symptoms of substance withdrawal by taking more of a previously experienced substance.

As used with respect to substance abuse, the term “preference” refers to the tendency to consume a substance that produces pleasurable effects, a opposed than a control substance that does not produce such effects (drug preference for alcohol can be tested, for example, by allowing an animal access to two bottles, one containing an alcohol solution, and one containing water and comparing the amount of each the animal consumes).

As used with respect to substance abuse, the term “seeking” refers to behavior aimed at obtaining a substance, even in the face of negative health and social consequences. Drug seeking, for example, is often uncontrollable and compulsive.

As used with respect to substance abuse, the terms “intake” or “consumption” refers to the amount of substance consumed by a subject (generally self-administered) over a selected period of time. Drug consumption, for example, is often uncontrollable and compulsive.

A “substance of abuse” includes any substance, the excessive consumption or administration of which can result in a symptom of substance abuse, dependence, or addiction as defined herein or substance dependence and abuse as defined by the current DSM Criteria promulgated by the American Psychiatric Association or equivalent criteria. Substances of abuse include, without limitation, an opioid, a psychostimulant, a sedative-hypnotic drug, a cannabinoid, an empathogen, a dissociative drug, alcohol, and nicotine. Thus, for example, morphine, heroin, cocaine, methamphetamines, barbiturates, cannabis (e.g. marijuana, hashish), 3-4 methylenedioxy-methamphetamine (MDMA), phencyclidine (PCP), ketamine, ethanol, and substances that mediate agonist activity at the dopamine D2 receptor are all drugs of abuse, as defined herein. Substances of abuse include, but are not limited to addictive drugs.

A “non-selective” modulator of a particular receptor or receptor subtype (e.g., an orexin receptor or a CRF receptor) is an agent that modulates other receptors and/or other receptor subtypes at the concentrations typically employed for modulation of the particular receptor or receptor subtype.

A “selective” modulator of a particular receptor or receptor subtype significantly modulates one or more of the normal functions of the particular receptor or receptor subtype at a concentration at which other receptors and/or receptor subtypes are not significantly modulated. Thus, a modulator can be selective for, e.g., an orexin receptor or can be selective for an orexin receptor subtype, such as, for example, the orexin receptor type 1 (OXR1).

A modulator “acts directly on” a receptor or its ligand when the modulator binds to the receptor or ligand, respectively.

A modulator “acts indirectly on” a receptor or its ligand when the modulator binds to a molecule other than the receptor or ligand, which binding results in modulation of receptor or ligand function, respectively.

An “inhibitor” or “antagonist” of a receptor is an agent that reduces, by any mechanism, any function of the receptor, as compared to that observed in the absence (or presence of a smaller amount) of the agent. An inhibitor of a receptor can affect: (1) the expression; mRNA stability; or protein trafficking, modification (e.g., phosphorylation), or degradation of a receptor or one or more of its subunits or of the ligand for the receptor, or (2) one or more of the normal activities of the receptor. An inhibitor of a receptor can be non-selective or selective. Preferred inhibitors (antagonists) are generally small molecules that act directly on, and are selective for, the target receptor.

An “enhancer” or “agonist” is an agent that increases, by any mechanism, any function of the receptor, as compared to that observed in the absence (or presence of a smaller amount) of the agent. An enhancer of a receptor can affect: (1) the expression; mRNA stability; or protein trafficking, modification (e.g., phosphorylation), or degradation of a receptor or one or more of its subunits or of the ligand for the receptor, or (2) one or more of the normal activities of the receptor. An enhancer of a receptor can be non-selective or selective. Preferred enhancers (agonists) are generally small molecules that act directly on, and are selective for, the target receptor.

The term “gene product” refers to a molecule that is ultimately derived from a gene. The molecule can be a polypeptide encoded by the gene, an mRNA encoded by a gene, a cDNA reverse transcribed from the mRNA, and so forth.

The terms “polypeptide,” “peptide” and “protein” are used interchangeably herein to refer to a polymer of amino acid residues. The terms apply to amino acid polymers in which one or more amino acid residue is an artificial chemical analogue of a corresponding naturally occurring amino acid, as well as to naturally occurring amino acid polymers.

The term “antibody,” as used herein, includes various forms of modified or altered antibodies, such as an intact immunoglobulin, an Fv fragment containing only the light and heavy chain variable regions, an Fv fragment linked by a disulfide bond (Brinkmann et al. (1993) Proc. Natl. Acad. Sci. USA, 90: 547-551), an Fab or (Fab)′2 fragment containing the variable regions and parts of the constant regions, a single-chain antibody and the like (Bird et al. (1988) Science 242: 424-426; Huston et al. (1988) Proc. Nat. Acad. Sci. USA 85: 5879-5883). The antibody may be of animal (especially mouse or rat) or human origin or may be chimeric (Morrison et al. (1984) Proc Nat. Acad. Sci. USA 81: 6851-6855) or humanized (Jones et al. (1986) Nature 321: 522-525, and published UK patent application #8707252).

The terms “binding partner,” or “capture agent,” or a member of a “binding pair” refers to molecules that specifically bind other molecules to form a binding complex such as antibody-antigen, lectin-carbohydrate, nucleic acid-nucleic acid, biotin-avidin, etc.

The term “specifically binds,” as used herein, when referring to a biomolecule (e.g., protein, nucleic acid, antibody, etc.), refers to a binding reaction which is determinative of the presence of the biomolecule in heterogeneous population of molecules (e.g., proteins and other biologics). Thus, under designated conditions (e.g. immunoassay conditions in the case of an antibody or stringent hybridization conditions in the case of a nucleic acid), the specified ligand or antibody binds to its particular “target” molecule and does not bind in a significant amount to other molecules present in the sample.

The terms “nucleic acid” or “oligonucleotide” or grammatical equivalents herein refer to at least two nucleotides covalently linked together. A nucleic acid of the present invention is preferably single-stranded or double-stranded and will generally contain phosphodiester bonds, although in some cases, as outlined below, nucleic acid analogs are included that may have alternate backbones, comprising, for example, phosphoramide (Beaucage et al. (1993) Tetrahedron 49(10):1925) and references therein; Letsinger (1970) J. Org. Chem. 35:3800; Sprinzl et al. (1977) Eur. J. Biochem. 81: 579; Letsinger et al. (1986) Nucl. Acids Res. 14: 3487; Sawai et al. (1984) Chem. Lett. 805, Letsinger et al. (1988) J. Am. Chem. Soc. 110: 4470; and Pauwels et al. (1986) Chemica Scripta 26: 141 9), phosphorothioate (Mag et al. (1991) Nucleic Acids Res. 19:1437; and U.S. Pat. No. 5,644,048), phosphorodithioate (Briu et al. (1989) J. Am. Chem. Soc. 111:2321, O-methylphophoroamidite linkages (see Eckstein, Oligonucleotides and Analogues: A Practical Approach, Oxford University Press), and peptide nucleic acid backbones and linkages (see Egholm (1992) J. Am. Chem. Soc. 114:1895; Meier et al. (1992) Chem. Int. Ed. Engl. 31: 1008; Nielsen (1993) Nature, 365: 566; Carlsson et al. (1996) Nature 380: 207). Other analog nucleic acids include those with positive backbones (Denpcy et al. (1995) Proc. Natl. Acad. Sci. USA 92: 6097; non-ionic backbones (U.S. Pat. Nos. 5,386,023, 5,637,684, 5,602,240, 5,216,141 and 4,469,863; Angew. (1991) Chem. Intl. Ed. English 30: 423; Letsinger et al. (1988) J. Am. Chem. Soc. 110:4470; Letsinger et al. (1994) Nucleoside & Nucleotide 13:1597; Chapters 2 and 3, ASC Symposium Series 580, “Carbohydrate Modifications in Antisense Research”, Ed. Y. S. Sanghui and P. Dan Cook; Mesmaeker et al. (1994), Bioorganic & Medicinal Chem. Lett. 4: 395; Jeffs et al. (1994) J. Biomolecular NMR 34:17; Tetrahedron Lett. 37:743 (1996)) and non-ribose backbones, including those described in U.S. Pat. Nos. 5,235,033 and 5,034,506, and Chapters 6 and 7, ASC Symposium Series 580, Carbohydrate Modifications in Antisense Research, Ed. Y. S. Sanghui and P. Dan Cook. Nucleic acids containing one or more carbocyclic sugars are also included within the definition of nucleic acids (see Jenkins et al. (1995), Chem. Soc. Rev. pp 169-176). Several nucleic acid analogs are described in Rawls, C & E News Jun. 2, 1997 page 35. These modifications of the ribose-phosphate backbone may be done to facilitate the addition of additional moieties such as labels, or to increase the stability and half-life of such molecules in physiological environments.

The terms “hybridizing specifically to” and “specific hybridization” and “selectively hybridize to,” as used herein refer to the binding, duplexing, or hybridizing of a nucleic acid molecule preferentially to a particular nucleotide sequence under stringent conditions. The term “stringent conditions” refers to conditions under which a probe will hybridize preferentially to its target subsequence, and to a lesser extent to, or not at all to, other sequences. Stringent hybridization and stringent hybridization wash conditions in the context of nucleic acid hybridization are sequence dependent, and are different under different environmental parameters. An extensive guide to the hybridization of nucleic acids is found in, e.g., Tijssen (1993) Laboratory Techniques in Biochemistry and Molecular Biology—Hybridization with Nucleic Acid Probes part I, chapt 2, Overview of principles of hybridization and the strategy of nucleic acid probe assays, Elsevier, N.Y. (Tijssen). Generally, highly stringent hybridization and wash conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Very stringent conditions are selected to be equal to the Tₘ for a particular probe. An example of stringent hybridization conditions for hybridization of complementary nucleic acids which have more than 100 complementary residues on an array or on a filter in a Southern or northern blot is 42° C. using standard hybridization solutions (see, e.g., Sambrook (1989) Molecular Cloning: A Laboratory Manual (2nd ed.) Vol. 1-3, Cold Spring Harbor Laboratory, Cold Spring Harbor Press, NY, and detailed discussion, below), with the hybridization being carried out overnight. An example of highly stringent wash conditions is 0.15 M NaCl at 72° C. for about 15 minutes. An example of stringent wash conditions is a 0.2×SSC wash at 65° C. for 15 minutes (see, e.g., Sambrook supra for a description of SSC buffer). Often, a high stringency wash is preceded by a low stringency wash to remove background probe signal. An exemplary medium stringency wash for a duplex of, e.g., more than 100 nucleotides, is 1×SSC at 45° C. for 15 minutes. An example of a low stringency wash for a duplex of, e.g., more than 100 nucleotides, is 4× to 6×SSC at 40° C. for 15 minutes.

The term “test agent” refers to an agent that is to be screened in one or more of the assays described herein. The agent can be virtually any chemical compound. It can exist as a single isolated compound or can be a member of a chemical (e.g. combinatorial) library. In a particularly preferred embodiment, the test agent will be a small organic molecule.

The term “small organic molecule” refers to a molecule of a size comparable to those organic molecules generally used in pharmaceuticals. The term excludes biological macromolecules (e.g., proteins, nucleic acids, etc.). Preferred small organic molecules range in size up to about 5000 Da, more preferably up to 2000 Da, and most preferably up to about 1000 Da.

The term “database” refers to a means for recording and retrieving information. In preferred embodiments the database also provides means for sorting and/or searching the stored information. The database can comprise any convenient media including, but not limited to, paper systems, card systems, mechanical systems, electronic systems, optical systems, magnetic systems or combinations thereof. Preferred databases include electronic (e.g. computer-based) databases. Computer systems for use in storage and manipulation of databases are well known to those of skill in the art and include, but are not limited to “personal computer systems”, mainframe systems, distributed nodes on an inter- or intra-net, data or databases stored in specialized hardware (e.g. in microchips), and the like.

The phrase “expression or activity of a gene” (e.g. Tsp42Ee gene) refers to the production of a gene product (e.g. the production of an mRNA and/or a protein) or to the activity of a gene product (i.e., the activity of a protein encoded by the gene).

The term “expression” refers to protein expression, e.g., mRNA and/or translation into protein. The term “activity” refers to the activity of a protein. Activities include but are not limited to phosphorylation, signaling activity, activation, catalytic activity, protein-protein interaction, transportation, etc. The expression and/or activity can increase or decrease. Expression and/or activity can be activated or inhibited directly or indirectly.

A “CRF, and/or CRF-BP, and/or CRF2 nucleic acid or polypeptide” refers to a polypeptide that is CRF, CRF-BP or CRF2 and/or to fragments thereof and/or to nucleic acids that encode the CRF, and/or CRF-BP, and/or CRF2 and/or to nucleic acids derived therefrom.

An “orexin and/or orexin receptor (e.g., OXR1) nucleic acid or polypeptide” refers to a polypeptide that is orexin or an orexin receptor and/or to fragments thereof and or to nucleic acids that encode the orexin and/or orexin receptor and/or to nucleic acids derived therefrom.

The term “detecting,” particularly when used with reference to electrophysiological methods includes, but is not limited to recording an electrophysiological signal from one or more cells.

The phrase “in conjunction with” when used in reference to the use of orexin receptor agonists or antagonists and CRF receptor antagonists or agonists indicates that the orexin receptor agonist/antagonist and the CRF receptor agonist/antagonist are administered so that there is at least some chronological overlap in their physiological activity on the organism. Thus the orexin receptor agonist/antagonist and the CRF receptor agonist/antagonist can be administered simultaneously and/or sequentially. In sequential administration there may even be some substantial delay (e.g., minutes or even hours or days) before administration of the second agent as long as the first administered agent has exerted, or is exerting, some physiological alteration on the organism when the second administered agent is administered or becomes active in the organism.

### BRIEF DESCRIPTION OF THE DRAWINGS

FIG. 1A-F. Orexin A potentiates NMDAR-mediated synaptic transmission in VTA dopamine neurons. (A) Orexin A (100 nM) increases NMDAR EPSCs evoked at +40 mV (n=12, 10/12 neurons significantly potentiated). Inset shows representative traces of NMDAR EPSCs before and 25 min after application of orexin A. Scale bars, 50 pA, 50 ms. (B) Orexin A dose-dependently increases NMDAR EPSCs. Each bar represents the mean and s.e.m. of EPSCs over a period of 2 min, 25 min after application of orexin A at concentrations of 1 (n=6, p<0.05), 10 (n=6, p<0.001), and 100 nM (n=6; p<0.001, one-way ANOVA, peak concentration effect versus baseline). (C) Orexin A-mediated increase in NMDAR EPSCs is blocked by the selective OXR1 antagonist SB 334857 (1 μM; n=8). (D) Orexin A (100 nM) does not increase AMPAR EPSCs evoked at −70 mV (n=8). Inset shows representative traces of AMPAR EPSCs before and 25 min after application of orexin A. Scale bars 20 pA, 20 ms. (E) After a 30-min application, orexin A (100 nM) increases NMDAR EPSCs for a prolonged period of time (n=6). (F) Orexin A increases NMDAR EPSCs in TH-positive cells (7/8 TH+ neurons were orexin A responders, 1/8 TH+ did not respond, and 2/2 were TH− and non-responders). TH was labeled with anti-tyrosine hydroxylase antibodies and FITC. Biocytin was labeled with streptavidin-conjugated Texas Red.

FIG. 2A-F. Orexin A potentiates NMDAR EPSCs via activation of phospholipase C and protein kinase C. (A) Intracellular application of the phospholipase C inhibitor, U73122 (filled circles, 1 μM, n=6) or chelerythrin, a protein kinase C inhibitor (open circles, 1 μM, n=6), blocked orexin A-mediated potentiation of NMDAR EPSCs. (B) An example trace of NMDAR EPSCs recorded in the presence of chelerythrin. (C) An example recording of NMDAR EPSCs in the presence of U73122. Intracellular application of the protein kinase A inhibitor, PKI (filled squares, 20 μM, n=8) or the cAMP inhibitor, Rp-cAMPS (open squares, 100 μM, n=5) did not block orexin A-mediated potentiation of NMDAR EPSCs. (E) An example trace of NMDAR EPSCs recorded in the presence of rp-cAMPS. (F) An example recording of NMDAR EPSCs in the presence of PKI.

FIG. 3A-F. Orexin A potentiation of NMDAR synaptic transmission is mostly due to alteration of NR2A subunits. (A) After bath application of NVP-AAM077 (0.4 μM), orexin A-mediated potentiation of NMDAR EPSCs was 20±6% (n=7). (B) Expression of orexin A-mediated potentiation of NMDAR EPSCs was reversed by application of NVP-AAM077 (n=6). (C) Orexin A-mediated potentiation was mostly blocked in the presence of the selective NR2A inhibitor, Zn²⁺ (300 nM, n=5). (D) Orexin A-mediated potentiation was not affected after bath application of the NR2B subunit selective antagonist, ifenprodil (3 μM; n=6). (E) After NVP-AAM077 (0.4 μM) and ifenprodil (3 μM) co-application, orexin A (100 nM) potentiation of NMDAR EPSCs was blocked (n=6). (F) After co-application of NVP-AAM077 (0.4 μM) and ifenprodil (3 μM), PPDA (1 μM) further inhibited NMDAR current, and blocked orexin A- (100 nM) mediated NMDAR potentiation (n=6).

FIG. 4A-D. Orexin A stimulates movement of NMDAR to the synapse. (A) Activity-dependent NMDAR antagonist, MK-801 (10 μM) was applied to VTA cells in the absence of stimulation until bath concentration equilibrated. The stimulator was switched on and MK-801 progressively blocked NMDAR EPSCs. Once EPSCs were blocked, stimulation was ceased for 40 min while MK-801 was washed out. Application of orexin A (100 nM, filled squares, n=6) during this washout period induced a significant potentiation of NMDAR-mediated EPSCs (control: open circles, n=5, p<0.05, t-test comparing averaged data from 61-67 min of control to orexin-treated cells). (B) An example recording of EPSCs during and after the application of MK-801. (C) MK-801 (20 μM) was co-applied with NMDA (500 μM) in the absence of stimulation to block both synaptic and extrasynaptic receptors. When the stimulator was turned on, NMDAR currents were fully blocked. MK-801 and NMDA were washed out in the absence of stimulation. Application of orexin A (100 nM, filled circles, n=6) during this washout period induced a significant potentiation of NMDAR-mediated EPSCs compared to controls (open circles, n=6, p<0.05). (D) An example recording of NMDAR EPSCs during and after co-application of MK-801 and NMDA.

FIG. 5A-B. OXR1 antagonist, SB 334867 blocks cocaine-associated plasticity in the VTA. (A) Rats were treated for 5 days with i.p. injections of either saline (open bars) or cocaine (closed bars, 15 mg/kg). Cocaine treatment (n=4) increased the AMPAR/NMDAR ratio compared to the saline-treated rats (n=4, p<0.05, t-test). In the presence of pre-administered SB 334867 (10 mg/kg, i.p.), cocaine-induced enhancement of the AMPAR/NMDAR ratio, measured in VTA slices 24 hrs later, was blocked by SB 334867 (saline, n=5; cocaine, n=11, p>0.05, t-test). (B) Orexin A (100 nM) was bath-applied for 5 min and slices were recorded 15 min later or at 3-4 hours after orexin application. AMPAR/NMDAR ratio was significantly increased 3-4 hours after orexin A application (n=8) compared to control (n=8) or 15 min after orexin A exposure (n=8, p<0.01). Bars represent mean and s.e.m.

FIG. 6A-I. Orexin A causes late-phase AMPAR-mediated plasticity. Orexin A (100 nM) was bath applied for 5 min then recorded 15 min or 3-4 hours after orexin A application. Example traces of mEPSCs and averaged mEPSCs from the same cell are shown (A) without orexin. (B) 15 min after or (C) 3-4 hrs after orexin A application. Scale bars, 20 pA, 100 ms for mEPSC traces and 5 pA, 5 ms for averaged mEPSCs. (D) Neurons recorded 3-4 hours after orexin A application had increased AMPAR mEPSC amplitude (n=7, filled bars) compared to 15 min after (n=6, shaded bars) or controls (n=7, open bars, p<0.05). The orexin-mediated increase in amplitude at 3-4 hours was blocked when APV (50 μM) was applied 3 min prior to and during orexin application (n=7, dotted bars, p>0.05). (E) AMPAR mEPSC frequency increased in neurons recorded 3-4 hours after orexin A application (n=7) compared to 15 min after (n=6) or control (n=7) conditions (p<0.05). AMPAR mEPSC frequency at 3-4 hours was also elevated with prior APV treatment (n=7, p<0.05). (F) A cumulative probability plot of amplitude for mEPSCs from example cells in control (black), 15 min (red), 3-4 hours (blue), or 3-4 hours after orexin A (100 nM) application with prior APV (50 μM) treatment (green). (G) A cumulative probability of frequency plot for mEPSCs from example cells in control, 15 min, 3-4 hours, or 3-4 hours after orexin application with prior APV (50 μM) treatment (green). (H) Changes in holding current were measured after bath application of AMPA (2 μM) in the presence of cyclothiazide (100 μM). There was no change in current when AMPA (2 μM) was superfused onto slices for 30 s between controls (n=9) and neurons recorded 15 min after orexin A application (n=5, p>0.05). (I) Bath-application of AMPA (2 μM) elicited a greater inward current in neurons recorded 3-4 hours after orexin A application compared to controls (n=6 p<0.05).

FIG. 7A-I. Orexin A causes an early increase in NMDAR short-term plasticity. Orexin A (100 nM) was bath-applied for 5 min then recorded 15 min or 3-4 hours after orexin A application. NMDAR mEPSCs were recorded in Mg²⁺-free aCSF with 20 μM glycine, 10 μM CNQX and 500 μM lidocaine when the neurons were voltage-clamped at −40 mV. Example traces of mEPSCs were recorded (A) without orexin, (B) 15 min after or (C) 3-4 hours after orexin A application. Scale bars, 50 pA, 100 ms. Below each condition is an averaged mEPSC from each of the above example neurons. Scale bars, 5 pA, 5 ms. (D) Orexin A increased NMDAR mEPSC amplitude 15 min after orexin A (100 nM) application (n=8) compared to controls (n=8) or 3-4 hours after orexin A application (n=7; p<0.05). (E) NMDAR mEPSC frequency was not altered by orexin A (100 nM) application (p>0.05). (F) A cumulative probability of amplitude plot for NMDAR mEPSCs from a single cell in control (black), 15 min (red) or 3-4 hours (blue) after orexin application. (G) A cumulative probability of frequency plot for mEPSCs from a single cell in control (black), 15 min (red) or 3-4 hours (blue) after orexin application. (H) Changes in holding current were measured after NMDA (50 μM, 30s) was bath-applied to VTA slices. NMDA-induced current was significantly greater in neurons 15 min after orexin A treatment (n=7) compared to controls (n=9, p<0.05). (I) There was no difference in NMDA-induced current between controls and neurons 3-4 hours after orexin A treatment (n=6, p>0.05).

FIG. 8A-E. OXR1 antagonist, SB 334867 administered i.p. or intra-VTA blocks the development of locomotor sensitization to cocaine. (A) Locomotor activity was assessed after i.p. administration of cocaine (squares, 15 mg/kg) or saline (circles) injections with SB 334867 (open symbols, 10 mg/kg, i.p.) or vehicle (closed symbols, n=13 for all groups to day 5). Vehicle-treated rats were injected with SB 334867 (10 mg/kg) on day 6 (open circles, saline+SB 334867, n=7; open squares, cocaine+SB 334867, n=7). (B) Rats received intra-VTA SB 334867 (6 μg/0.3 μl; open symbols) or vehicle (closed symbols) and were tested for locomotor activity after receiving i.p. injections of cocaine (squares: SB 334867, n=12; vehicle, n=12) on days 1-7. Control rats received saline (circles: SB 334867, n=10; vehicle, n=8). (C,D) Individual rats' locomotor activity on days 1 and 7 for cocaine-treated rats in the presence (C) or absence (D) of intra-VTA SB 334867. Arrows indicate mean locomotor distance. (E) Reconstructed SB 334967 injection sites in the VTA are shown in coronal sections. Distance from bregma is shown to the right of each section (in mm).

FIG. 9 shows that CRF (6-33) significantly reduced limited access 10% ethanol voluntary consumption Results are presented as g/kg of ethanol consumed in a 1 hr period, n=14). See Example 2 for details.

FIG. 10 indicates that CRF (6-33) did not reduce water consumption. Results are presented as mL of ethanol consumed in a 24 hr period following infusion (n=14).

FIG. 11A-D shows that the OXR1 antagonist SB 334867 reduced cocaine reinforcement. Rats were trained to lever press for cocaine (0.5 mg/infusion) on an FR1 or FR3 schedule, and subsequently a progressive ratio schedule. Rats were given vehicle on the 2nd and 3rd day of progressive ratio testing and then SB 334867 (10 mg/kg, i.p.) on the 4th day. A, Natural log of active lever presses for cocaine was reduced after SB334867 administration (p<0.01, n=12). B, Raw active lever presses was reduced after SB334867 administration (p<0.05, n=12). C, The total number of cocaine infusions was decreased after SB334867 administration (p<0.01, n=12). D, The breakpoint was reduced after SB334867 administration (p<0.05, n=12). See Example 2 for details.

FIG. 12A-D shows that the OXR1 antagonist SB 334867 did not alter reinforcement for food. Rats were trained to lever press for food on an FR1 or FR3 schedule, and subsequently a progressive ratio schedule. Rats were given vehicle on the 2nd and 3rd day of progressive ratio testing and then SB 334867 (10 mg/kg, i.p.) on the 4th day. A, Natural log of active lever presses for food (p<0.05, n=10). B, Raw active lever presses (p<0.05, n=10). C, The total number of food pellets received was not altered after SB334867 administration (p<0.05, n=10). D, The breakpoint was unaltered after SB334867 administration (p<0.05, n=10).

FIG. 13 shows that CRF increased NMDAR EPSCs in a concentration dependent manner in mice. There was no potentiation of NMDARs after application of 10 nM CRF. Ungless et al., 2003 Neuron 39: 401-7.

FIG. 14A-D shows that orexin A potentiated the effect of CRF in rats. A, Orexin A at 1 nM potentiated NMDARs 5.7±1.6% (n=6). B, An example trace of a 5 min application of CRF (1 μM) on NMDAR eEPSCs in rats. C, An example trace of NMDAR eEPSCs after a 5 min co-application of orexin A (1 nM) with CRF (10 nM). D, Application of orexin A (1 nM) with CRF (10 nM) significantly potentiated NMDAR eEPSCs to a maximum of 123±12% (p<0.05, n=7).

### DETAILED DESCRIPTION

This invention pertains to the discovery that orexin receptor modulates N-methyl-D-aspartate (NMDA) currents and orexin receptor antagonists inhibit cocaine locomotor sensitization. Because of the NMDA modulation, this has broad implications for Parkinsons, Alzheimers, cognition, learning, addiction etc.

In addition, this invention pertains to the discovery that CRF increases NMDAR (N-methyl-D-aspartate receptor)-mediated currents at excitatory synapses onto a subset of dopamine cells in the ventral tegmental area (VTA) in the mammalian brain. This effect is not blocked by a CRF receptor 1 (CRF-R1) antagonist, but is blocked by a CRF receptor 2 (CRF-R2) antagonist. It was also discovered that an inhibitor of the CRF-binding protein (CRF-BP) blocks the effects of CRF, which indicates that CRF-BP, rather than inactivating “free” CRF, is necessary for CRF to potentiate NMDAR currents. Accordingly, Urocortin, which may be the endogenous CRF-R2 ligand and also binds CRF-BP, mimics CRF, while ovine CRF and Urocortin II, which do not bind CRF-BP, do not potentiate NMDAR currents. These results provide specific roles for CRF-R2 and CRF-BP in the modulation of neuronal activity.

In the treatment of alcohol abuse or other substance abuse, Alzheimer's disease, Parkinson's disease, etc., agonists or anatagonists of the orexin receptor and/or the CRF pathway can be administered to reduce or prevent one or more symptoms or behaviors associated with the pathology. Thus, for example, an orexin receptor antagonist can be used to reduce or prevent one or more behaviors associated with substance abuse. Such agents can be used, for example, in the treatment of substance abuse (e.g., self-administration of substances of abuse) and/or withdrawal from substances of abuse, and various neurological conditions characterized by overactivation, inactivation, and/or loss of dopinergic neurons (e.g. Alzheimer's disease, Parkinson's disease, etc.).

In various embodiments, orexin receptor modulators (e.g., agonists/antagonists) and modulators of the CRF pathway can be administered in conjunction with each other to affect NMDA activity while minimizing adverse side effects.

Other indications include, but are not limited to depression; anxiety; addictions; obsessive compulsive disorder; affective neurosis/disorder; depressive neurosis/disorder; anxiety neurosis; dysthymic disorder; behaviour disorder; mood disorder; sexual dysfunction; psychosexual dysfunction; sex disorder, sexual disorder, schizophrenia; manic depression; delerium; dementia; severe mental retardation and dyskinesias such as Huntington's disease and Gilles de la Tourett's syndrome; disturbed biological and circadian rhythms; feeding disorders, such as anorexia, bulimia, cachexia, and obesity; diabetes; appetite/taste disorders; vomiting/nausea; asthma; cancer; Parkinson's disease; Cushing's syndrome/disease; basophil adenoma; prolactinoma; hyperprolactinemia; hypopituitarism; hypophysis tumor/adenoma; hypothalamic diseases; Froehlich's syndrome; adrenohypophysis disease; hypophysis disease; hypophysis tumor/adenoma; pituitary growth hormone; adrenohypophysis hypofunction; adrenohypophysis hyperfunction; hypothalamic hypogonadism; Kallman's syndrome (anosmia, hyposmia); functional or psychogenic amenorrhea; hypopituitarism; hypothalamic hypothyroidism; hypothalamic-adrenal dysfunction; idiopathic hyperprolactinemia; hypothalamic disorders of growth hormone deficiency; idiopathic growth hormone deficiency; dwarfism; gigantism; acromegaly; and sleep disturbances associated with such diseases as neurological disorders, neuropathic pain and restless leg syndrome, heart and lung diseases; acute and congestive heart failure; hypotension; hypertension; urinary retention; osteoporosis; angina pectoris; myocardial infarction; ischaemic or haemorrhagic stroke; subarachnoid haemorrhage; head injury such as subarachnoid haemorrhage associated with traumatic head injury; ulcers; allergies; benign prostatic hypertrophy; chronic renal failure; renal disease; impaired glucose tolerance; migraine; hyperalgesia; pain; enhanced or exaggerated sensitivity to pain, such as hyperalgesia, causalgia and allodynia; acute pain; burn pain; atypical facial pain; neuropathic pain; back pain; complex regional pain syndromes I and II; arthritic pain; sports injury pain; pain related to infection, e.g. HIV, post-polio syndrome, and post-herpetic neuralgia; phantom limb pain; labour pain; cancer pain; post-chemotherapy pain; post-stroke pain; post-operative pain; neuralgia; conditions associated with visceral pain including irritable bowel syndrome, migraine and angina; urinary bladder incontinence e.g. urge incontinence; tolerance to narcotics or withdrawal from narcotics; sleep disorders; sleep apnea; narcolepsy; insomnia; parasomnia; jet-lag syndrome; and neurodegenerative disorders, which includes nosological entities such as disinhibition-dementia-parkinsonism-amyotrophy complex; pallido-ponto-nigral degeneration, epilepsy, and seizure disorders.

In certain embodiments, this invention provides methods of screening for agent(s) that modulate orexin and/or corticotrophin-releasing factor (CRF) potentiation of N-methyl-D-aspartate receptor (NMDAR) mediated currents. The methods typically involve contacting a cell, tissue or organism with one or more test agents and detecting the activity or expression of an orexin receptor and/or a CRF2 receptor, where an alteration of expression or activity of the orexin and/or CRF2 receptor as compared to a control indicates that the test agent is an agent that modulates orexin and/or CRF potentiation of NMDAR-mediated currents and is a good candidate compound for use in the treatment of substance abuse, withdrawal, and a variety of other conditions, e.g. as described herein.

CRF binding protein (CRF-BP), rather than inactivating ‘free’ CRF, is necessary for CRF to potentiate NMDAR currents and this potentiation is mediated via the CRF2 receptor, not the CRF1 receptor. Thus, CRF and CRF-BP both appear to be required to activate/potentiate NMDA receptors. Thus, the interaction between these three components (CRF, CRF-BP, and the CRF2 receptor) also provide effective targets for screening for modulators of NMDA potentiation.

Without being bound to a particular theory, it is believed that the interaction of orexin receptor activity and NMDA receptor activity provides a previously unknown link between orexin and dopamine activity.

Similarly, it is believed that the CRF/CRF-BP/CRF2R interaction provides a link between CRF and dopamine activity. It is believed that CRF2R agonists can provide a therapeutic modality for Parkinson's and/or Alzheimers disease (or other related pathologies) by activating NMDA receptors. In certain embodiments, such agonists will directly agonize/activate CRF2 receptors. In certain other embodiments, such agonists will act by binding both CRF2 receptors and CRF-BP.

Such CRF2R agonists or antagonists can be identified by screening for the ability to upregulate or inhibit expression or activity of the CRF2 receptor and/or CRF and/or CRF-BP and or the interaction of these components (e.g. by binding CRF, CRF-BP, CRF2R or a complex of two or more of these proteins).

I. Modulating NMDA Currents by Altering Expression, Activity, and/or Interaction of Orexin, Orexin Receptor, CRF, CRF-BP, and/or CRF Receptor.

The invention provides methods for modulating NMDAR-mediated currents by altering expression, activity, and/or interaction of orexin, orexin receptor, CRF, CRF-BP, and/or CRF receptor. Such methods may be carried out in vivo, for example, in prophylactic or therapeutic methods aimed at preventing or mitigating one or more symptoms of conditions amenable to treatment by modulating NMDA currents or to enhance performance in areas such as, e.g., cognition and learning. The methods of the invention are also useful in vivo, in standard animal model systems in studies aimed at furthering our understanding of modulation of dopaminergic neurotransmission, synaptic plasticity, and dopamine-mediated motivational behaviours. Alternatively, methods of the invention can be carried out in vitro, for example, in assays to elucidate interactions among various signalling systems with respect to dopaminergic neurotransmission and synaptic plasticity. In preferred in vivo methods, the subject is a mammal that is not being treated for an eating disorder. In particular embodiments, the subject is a mammal that is not being treated for a sleep/wakefulness disorder.

Accordingly, in one embodiment, the invention provides a method of modulating an NMDAR-mediated current that entails administering to a mammal, an orexin receptor agonist or antagonist in a concentration sufficient to alter the NMDAR-mediated current.

A) Method of Mitigating a Symptom of Substance Abuse.

In a particular embodiment of the invention, an orexin receptor agonist or antagonist is administered to a mammal in a concentration sufficient to reduce or prevent a symptom of substance abuse. In certain embodiments, an orexin receptor antagonist is administered to reduce or prevent a symptom of substance abuse. The antagonist can be non-selective with respect to orexin receptor subtype or can be selective for a particular orexin receptor subtype. In particular embodiments, an antagonist of the orexin receptor type 1 (OXR1) is employed to treat substance abuse.

This method can be used to treat any form of substance abuse in which NMDA currents play a role, including, but not limited to, abuse of opioids, sedative-hypnotics, psychostimulants, cannabinoids, empathogens, alcohol, and nicotine. Exemplary opioids include morphine, codeine, heroin, butorphanol, hydrocodone, hydromorphone, levorphanol, meperidine, nalbuphine, oxycodone, fentanyl, methadone, propoxyphene, remifentanil, sufentanil, and pentazocine. Sedative-hypnotics include, for example, benzodiazepines and barbiturates. Exemplary, benzodiazepines include, without limitation, alprazolam, chlordiazepoxide, chlordiazepoxide hydrochloride, chlormezanone, clobazam, clonazepam, clorazepate dipotassium, diazepam, droperidol, estazolam, fentanyl citrate, flurazepam hydrochloride, halazepam, lorazepam, midazolam hydrochloride, oxazepam, prazepam, quazepam, temazepam, and triazolam. Exemplary barbiturates include amobarbital, amobarbital sodium, aprobarbital, butabarbital sodium, hexobarbital sodium, mephobarbital, metharbital, methohexital sodium, pentobarbital, pentobarbital sodium, phenobarbital, phenobarbital sodium, secobarbital, secobarbital sodium, talbutal, thiamylal sodium, thiopental sodium, and the like. Psychostimulants include drugs that stimulate the central nervous system, such as, for example, amphetamine, cocaine, methamphetamine, methylphenidate (ritalin), and methylene dioxy-methamphetamine (MDMA). Exemplary cannabinioids include tetrahydrocannabinol (THC), dronabinol, and arachidonylethanolamide (anandamide, AEA). Empathogens include phenethylamines, such as, for example, MDMA, 3,4-methylenedioxy amphetamine (MDA), 3,4-methylenedioxy-N-ethylamphetamine (MDEA), 2,5-Dimethoxy-4-iodo-phenethylamine or 1-(2,5-dimethoxy-4-iodophenyl)-2-aminoethane (2C-I), 2,5-dimethoxy-4-bromo-phenethylamine (2C-B), and N-methyl-1-(3,4-methylenedioxyphenyl)-2-butanamine. Dissociative drugs include PCP and ketamine.

Examples of symptoms of substance abuse that are amenable to treatment in this manner include reward, incentive salience, craving, preference, seeking, and/or intake (self-administration) of said substance of abuse; relapse; and a symptom of withdrawal. Such symptoms can be treated during substance dependence or during withdrawal. For example, an orexin receptor antagonist can be administered to inhibit/modulate the consumption of, or to reduce the rewarding properties of, substances of abuse during drug dependence. Such antagonists are useful for reducing substance abuse-specific stress and anxiety associated with initial abstinence or withdrawal from substance abuse. Orexin receptor antagonists can also be administered to maintain abstinence from drug seeking following rehabilitation and/or to prevent substance abuse reinstatement. In this case, the treatment can be combined with appropriate behavioral therapy. In particular, orexin receptor antagonists can improve cognition/decision-making such that the otherwise uncontrollable urge to retake substances of abuse is reduced. Orexin receptor antagonists can also be employed to prevent substance abuse in individuals determined to be susceptible to substance abuse. For example, orexin receptor antagonists can inhibit the motivation of susceptible individuals to seek substances of abuse.

B) Method Modulating a NMDAR-Mediated Current in a Dopaminergic Neuron.

The invention also provides a method modulating a NMDAR-mediated current in a dopaminergic neuron that entails modulating binding between orexin and the orexin receptor type 1 (OXR1). As used herein, “modulating binding” encompasses inhibiting binding, as well as enhancing binding of the OXR1 receptor by orexin or an orexin agonist that acts directly on the OXR1 receptor.

C) Co-Administration of Orexin and CRF Modulators.

As CRF, like orexin, modulates NMDAR-mediated currents in a dopaminergic neuron, CRF and orexin action can be modulated in conjuction to provide a desired degree of modulation of NMDAR-mediated currents. Accordingly, in one embodiment, the invention provides a method of modulating a NMDAR-mediated current in a mammal that entails administering to the mammal an orexin receptor agonist or antagonist in conjunction with a CRF receptor agonist or antagonist. In a particular embodiment, an orexin receptor agonist or antagonist can be administered in conjunction with a CRF receptor agonist or antagonist, wherein the concentrations of agents administered are sufficient to reduce or prevent a symptom of substance abuse. The invention also provides a method of modulating the activity of CRF on a dopaminergic neuron that entails modulating binding between orexin and OXR1, e.g., using a direct orexin receptor agonist or antagonist.

In any of these co-administration methods, (1) an orexin receptor antagonist can be administered in conjunction with a CRF receptor antagonist; (2) an orexin receptor antagonist can be administered in conjunction with a CRF receptor agonist; (3) an orexin receptor agonist can be administered in conjunction with a CRF receptor antagonist, and (4) an orexin receptor agonist can be administered in conjunction with a CRF receptor agonist, depending on the degree of modulation of NMDAR-mediated current desired and other considerations (such as the condition being treated, other effects of the orexin and/or CRF agonist and/or antagonist, etc.). More specifically, orexin receptor agonists potentiate, and antagonists inhibit, NMDAR-mediated currents. Similarly, CRF receptor agonists potentiate, and antagonists inhibit, NMDAR-mediated currents. To treat certain symptoms of substance abuse, it is advantageous to reduce NMDAR-mediated currents and thus treatment with an orexin receptor antagonist and/or a CRF receptor antagonist is indicated. Co-administration of the two antagonists can allow the use of lower doses of the antagonists than would be required if either were administered alone, which can reduce undesirable side effects. As used herein, the term “CRF receptor agonists/antagonists” include those agents that via effects on CRF-BP. Thus, for example, CRF receptor agonists include agents that increase CRF-BP, and CRF receptor antagonists include agents that decrease CRF-BP.

Agents that act via effects on orexin or CRF (including CRF-BP) action can be co-administered by simultaneous administration or sequential administration. In the case of sequential administration, the first administered agent must have exerted, or be exerting, some physiological alteration on the organism when the second administered agent is administered or becomes active in the organism.

D) Modulators of Orexin and CRF.

1) In General.

Any orexin receptor and/or CRF receptor agonist and/or antagonist can be employed in the methods of the invention, provided that any agonist/antagonist employed in vivo should be sufficiently well tolerated to allow its use for the intended purpose.

Orexin, orexin receptor, CRF, CRF-BP, and/or CRF receptor expression can enhanced or inhibited using a wide variety of approaches known to those of skill in the art. For example, methods of inhibiting expression include, but are not limited to, antisense molecules, target-specific ribozymes, target-specific catalytic DNAs, intrabodies directed against target proteins, RNAi, gene therapy approaches that knock out orexin, orexin receptor, CRF, CRF-BP, and/or CRF, and small organic molecules that inhibit expression of the target gene(s).

Orexin, orexin receptor, CRF, CRF-BP, and/or CRF receptor expression and/or activity, and/or interaction can be enhanced by introducing constructs encoding orexin, orexin receptor, CRF, CRF-BP, and/or CRF receptor into the cell (e.g. using gene therapy approaches) or upregulating endogenous expression of orexin, orexin receptor, CRF, CRF-BP, and/or CRF receptor (e.g., using agents identified in the screening assays of this invention).

In certain embodiments, orexin, orexin receptor, CRF, CRF-BP, and/or CRF receptor expression and/or activity and/or interaction can be inhibited by the use of small organic molecules (e.g., molecules identified according to the screening methods described herein). Such molecules include, but are not limited to, molecules that specifically bind to the DNA comprising the orexin, orexin receptor, CRF, CRF-BP, and/or CRF receptor promoter and/or coding region, molecules that bind to and complex with orexin, orexin receptor, CRF, CRF-BP, and/or CRF receptor mRNA, molecules that bind to orexin, orexin receptor, CRF, CRF-BP, and/or CRF receptor proteins and/or complexes thereof, and the like.

Thus, the agonist/antagonist can act directly on the orexin or CRF receptor or indirectly (e.g., by acting on CRF-BP).

2) Orexin Modulators.

Examples of direct orexin receptor antagonists include, but are not limited to, tetrahydroisoquinolines (see, e.g., Koberstein et al. (2003) Chimia 57: 270-275 [incorporated by reference herein in its entirety]; U.S. Pat. No. 6,703,392, issued Mar. 9, 2004 to Aissaoui et al. [incorporated by reference herein in its entirety]), aroyl piperazine derivatives (see, e.g., U.S. Patent Publication US 20040242575 [incorporated by reference herein in its entirety]), 1-(2-methylbenzoxazol-6-yl)-3-[1,5]naphthyridin-4-yl urea hydrochloride (SB-334867-A; see, e.g., Smart et al (2001) J. Pharmacol. 132 1179; Porter et al (2001) Bioorg. Med. Chem. Lett. 11 1907 [incorporated by reference herein in their entireties]), N-(6,8-difluoro-2-methyl-4-quinolinyl)-N′-[4-(dimethylamino)phenyl]urea (SN-408124; Langmead et al (2004) Br. J. Pharmacol. 141 340 [incorporated by reference herein in its entirety]), GW649868 (GlaxoSmithKline), phenyl urea derivatives, and phenyl thiourea derivatives (see, e.g., PCT Publication WO 2000/47577 [incorporated by reference herein in its entirety]). Tetrahydroisoquinoline orexin receptor antagonists include compounds having the structure: 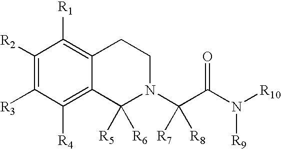
wherein:

R¹, R², R₃, R⁴ independently represent cyano, nitro, halogen, hydrogen, hydroxy, lower alkyl, lower alkenyl, lower alkoxy, lower alkenyloxy, trifluoromethyl, trifluoromethoxy, cycloalkyloxy, aryloxy, aralkyloxy, heterocyclyloxy, heterocyclylalkyloxy, R¹¹CO—, NR¹²R¹³CO—, R¹²R¹³N—, R¹¹OOC—, R¹¹SO₂NH— or R¹⁴—CO—NH—, or R² and R³ together as well as R¹ and R² together and R³ and R⁴ together may form with the phenyl ring a five, six or seven-membered ring containing one or two oxygen atoms;

R⁵ represents aryl, aralkyl, lower alkenyl, trifluoromethyl, cycloalkyl, heterocyclyl or heterocyclyl-lower alkyl;

R⁶ represents hydrogen, aryl, aralkyl, lower alkyl, lower alkenyl, trifluoromethyl, cycloalkyl, heterocyclyl or heterocyclyl-lower alkyl;

R⁷ and R⁸ independently represent hydrogen, aryl, aralkyl, lower alkyl, lower alkenyl, cycloalkyl, heterocyclyl or heterocyclyl-lower alkyl;

R⁹ represents aryl, aralkyl, lower alkyl, lower alkenyl, trifluoromethyl, cycloalkyl, heterocyclyl or heterocyclyl-lower alkyl;

R¹⁰ represents hydrogen, aryl, aralkyl, lower alkyl, lower alkenyl, trifluoromethyl, cycloalkyl, heterocyclyl or heterocyclyl-lower alkyl;

R¹¹ represents lower alkyl, aryl, aralkyl, heterocyclyl or heterocyclyl-lower alkyl;

R¹² and R¹³ independently represent hydrogen, alkyl, cycloalkyl, aryl, aralkyl, heterocyclyl or heterocyclyl-lower alkyl; and

R¹⁴ represents alkyl, aryl, cycloalkyl, heterocyclyl, R¹²R¹³N— or R¹¹O—, including optically pure enantiomers, mixtures of enantiomers, racemates, optically pure diastereoisomers, mixtures of diastereoisomers, diastereoisomeric racemates, mixtures of diastereolsomeric racemates, meso forms, and pharmaceutically acceptable salts thereof. Additional tetrahydroisoquinoline orexin receptor antagonists useful in the invention have the structure: 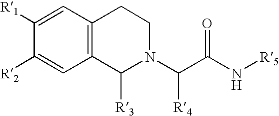
wherein:

R′¹ and R′² independently represent hydrogen, hydroxy, lower alkoxy or halogen or may form with the phenyl ring a five, six or seven membered-ring containing one or two oxygen atoms;

R′³ represents aryl, aralkyl, lower alkenyl, cycloalkyl, heterocyclyl or heterocyclyl-lower alkyl;

R′⁴ represents hydrogen, aryl, aralkyl, lower alkyl, lower alkenyl, cycloalkyl, heterocyclyl or heterocyclyl-lower alkyl; and

R′⁵ represents aryl, aralkyl, lower alkyl, lower alkenyl, cycloalkyl, heterocyclyl or heterocyclyl-lower alkyl;

including optically pure enantiomers, mixtures of enantiomers, racemates, optically pure diastereoisomers, mixtures of diastercoisomers, diastercoisomeric racemates, mixtures of diastereoisomeric racemates, meso forms, and pharmaceutically acceptable salts thereof. (See U.S. Pat. No. 6,703,392.)

Examples of direct orexin receptor agonists useful in the methods of the invention include, but are not limited to, orexin A, orexin B (see, e.g., Sakurai et al (1998) Cell 92 573 [incorporated by reference herein in its entirety]), and [Ala¹¹,D-Leu¹⁵]-orexin B (OXR2 agonist; see, e.g., Asahi et al (2003) Bioorg. Med. Chem. Lett. 13 111 [incorporated by reference herein in its entirety], Tocris). All of these orexin agonists are available from KOMA Biotech Inc., Seoul, Korea (http://www.komabiotech.com).

The orexin agonist/antagonist can be non-selective or selective for a particular receptor subtype (e.g., OXR1 or OXR2). In particular embodiments, the orexin receptor agonist or antagonist is selective for the orexin receptor type 1 (OXR1). Exemplary OXR1-selective antagonists include 1-(2-methylbenzoxazol-6-yl)-3-[1,5]naphthyridin-4-yl urea hydrochloride (SB-334867-A; Hayner et al (2000) Regul. Peptides 96 45 [incorporated by reference herein in its entirety], Banyu Pharmaceutical Co., Tocris) and N-(6,8-difluoro-2-methyl-4-quinolinyl)-N′-[4-(dimethylamino) phenyl]urea (SN-408124; Langmead et al (2004) Br. J. Pharmacol. 141 340 [incorporated by reference herein in its entirety]), both of which are available from KOMA Biotech Inc., Seoul, Korea (http://www.komabiotech.com).

Additional examples of OXR1-selective antagonists include morpholine derivatives (U.S. Pat. No. 6,943,160, issued Sep. 13, 2005 to Branch et al. [incorporated by reference herein in its entirety]), such as, fore example compounds having the structure: 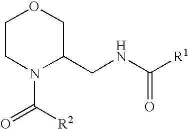
wherein:

R¹ is phenyl, naphthyl, a mono or bicyclic heteroaryl group containing up to 3 heteroatoms selected from N, O and S; any of which may be optionally substituted;

R² represents phenyl or a 5- or 6-membered heteroaryl group containing up to 3 heteroatoms selected from N, O and S, wherein the phenyl or heteroaryl group is substituted by R³, and further optional substituents; or R² represents an optionally substituted bicyclic aromatic or bicyclic heteroaromatic group containing up to 3 heteroatoms selected from N, O and S;

R³ represents an optionally substituted (C₁₋₄)alkoxy, halo, optionally substituted (C₁₋₆)alkyl, optionally substituted phenyl, or an optionally substituted 5- or 6-membered heterocyclic ring containing up to 3 heteroatoms selected from N, O and S; or a pharmaceutically acceptable salt thereof.

Other morpholine-based OXR1-selective antagonists include those having the structure:

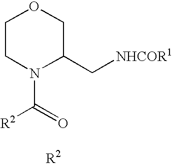

wherein R¹ and R² are selected from the following:

| R¹ | R ² |
|---|---|
| 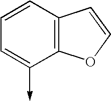 | 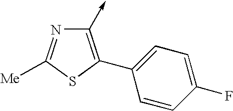 |
| 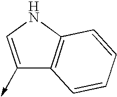 | 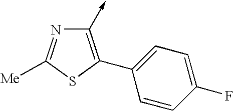 |
| 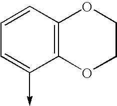 | 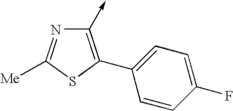 |
| 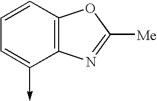 | 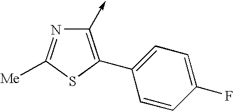 |
| 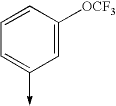 | 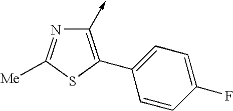 |
| 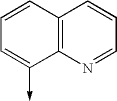 | 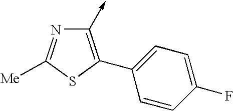 |
| 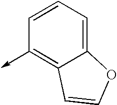 | 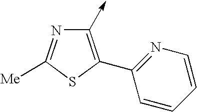 |
| 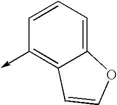 | 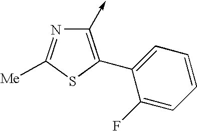 |
| 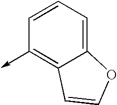 | 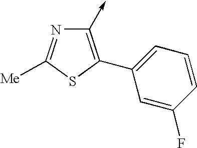 |
| 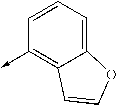 | 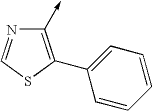 |

or a pharmaceutically acceptable salt of any one thereof.

Exemplary morpholine derivative useful in the invention include (RS)-3-(2-Methoxybenzamidomethyl)-4-((4-(2-methyl-5-phenyl)thiazolyl)carbonyl)morpholine, (RS)-3-((4-Benzofuranyl)carbonylaminomethyl)-4-((4-(2-methyl-5-(4-fluorophenyl))thiazolyl)carbonyl)morpholine, (RS)-3-(2-Methoxybenzamidomethyl)-4-((4-(2-methyl-5-(4-fluorophenyl))thiazolyl) carbonyl)morpholine, and pharmaceutically acceptable salts of any one thereof.

Other OXR1-selective antagonists are phenyl urea derivatives and phenyl thiourea derivatives (U.S. Pat. No. 6,699,879, issued Mar. 2, 2004 to Coulton et al.; U.S. Pat. No. 6,372,757, issued Apr. 16, 2002 to Johns et al.; U.S. Pat. No. 6,596,730, issued Jul. 22, 2003 to Coulton et al. [incorporated by reference herein in their entireties]). Examples include compounds having the structure: 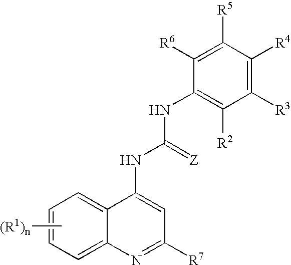
wherein:

Z represents oxygen or sulfur;

R¹ represents (C₁₋₆)alkyl, (C₂₋₆)alkenyl or (C₁₋₆)alkoxy, any of which may be optionally substituted; halogen, R⁸CO— or NR⁹R¹⁰CO—;

R², R³, R⁴, R⁵ and R⁶ independently represent (C₁₋₆)alkyl, (C₂₋₆)alkenyl, (C₁₋₆)alkoxy or (C₁₋₆)alkylthio, any of which may be optionally substituted; hydrogen, halogen, nitro, cyano, aryloxy, aryl(C₁₋₆)alkyloxy, aryl(C₁₋₆)alkyl, R⁸CO—, R⁸SO₂ NH—, R⁸SO₂O—, R⁸CON(R¹¹)—, NR⁹R¹⁰—, NR⁹R¹⁰CO—, —COOR⁹, R¹¹C(.dbd.NOR⁸), heterocyclyl or heterocyclyl(C₁₋₆)alkyl;

or an adjacent pair of R², R³, R⁴, R⁵ and R⁶ together with the carbon atoms to which they are attached form an optionally substituted carbocyclic or heterocyclic ring;

R⁷ is (C₁₋₆)alkyl, (C₂₋₆)alkenyl, (C₁₋₆)alkoxy or (C₁₋₆)alkylthio, any of which may be optionally substituted; halogen, hydroxy, nitro, cyano, NR⁹R¹⁰—, NR⁹R¹⁰CO—, N₃, —OCOR⁹ or R⁸CON(R¹¹)—;

R⁸ is (C₁₋₆)alkyl, (C₂₋₆)alkenyl, heterocyclyl, heterocyclyl(C₁₋₆)alkyl, heterocyclyl(C₂₋₆)alkenyl, aryl, aryl(C₁₋₆)alkyl or aryl(C₂₋₆)alkenyl, any of which maybe optionally substituted;

R⁹ and R¹⁰ independently represent hydrogen, (C₁₋₆)alkyl, (C₂₋₆)alkenyl, heterocyclyl, heterocyclyl(C₁₋₆)alkyl, aryl or aryl(C₁₋₆)alkyl, any of which maybe optionally substituted;

R¹¹ is hydrogen or (C₁₋₆)alkyl; and

n is 0, 1, 2, or 3;

or a pharmaceutically acceptable salt thereof. Examples of such compounds that are useful in the invention include 1-(2-methylbenzoxazol-6-yl)-3-(2-methylquinolin-4-yl)urea, 1-(4-dimethylaminophenyl)-3-(2-methylquinolin-4-yl)urea, 1-(2-methylbenzoxazol-6-yl)-3-(2-chloroquinolin-4-yl)urea, 1-(4-N,N-dimethylaminophenyl)-3-(2-chloroquinolin-4-yl)urea, 1-(3-butyryl-4-methoxyphenyl)-3-(5,8-difluoroquinolin-4-yl)urea, N-cyclopropylmethyl-5-[3-(8-fluoro-2-methyl-quinolin-4-yl)-ureido]-2-methoxy-benzamide hydrochloride, 1-(4-acetyl-phenyl)-3-(8-fluoro-2-methyl-quinolin-4-yl)-urea, 1-(6,8-difluoro-2-methyl-quinolin-4-yl)-3-(4-dimethylamino-phenyl)-urea, 1-(5,8-difluoro-2-methyl-quinolin-4-yl)-3-(5-oxo-5,6,7,8-tetrahydro-naphthalen-2-yl)-urea, N-cyclopropylmethyl-5-[3-(5,8-difluoro-2-methyl-quinolin-4-yl)-ureido]-2-methoxy-benzamide, 1-(3-chloro-4-methoxy-phenyl)-3-(5,8-difluoro-2-methyl-quinolin-4-yl)-urea, and pharmaceutically acceptable salts thereof.

Also useful as OXR1-selective antagonists are compounds having the structure: 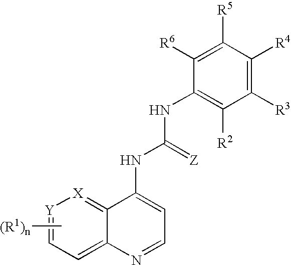
wherein:

one of X and Y is N and the other is CH;

Z represents oxygen or sulphur;

R¹ represents (C₁₋₆)alkyl, (C₂₋₆)alkenyl or (C₁₋₆)alkoxy, any of which may be optionally substituted; halogen, R⁷CO— or NR⁸R⁹CO—;

R², R³, R⁴, R⁵ and R⁶ independently represent (C₁₋₆)alkyl, (C₂₋₆)alkenyl, (C₁₋₆)alkoxy or (C₁₋₆)alkythio, any of which may be optionally substituted; hydrogen, halogen, nitro, cyano, aryloxy, aryl(C₁₋₆)alkyloxy, aryl(C₁₋₆)alkyl, R⁷CO—, R⁷SO₂NH—, R⁷CON(R¹⁰)—, NR⁸, R⁹—, NR⁸R⁹CO—, —COOR⁸, heterocyclyl or heterocyclyl(C₁₋₆)alkyl;

or an adjacent pair of R², R³, R⁴, R⁵ and R⁶ together with the carbon atoms to which they are attached form an optionally substituted carbocyclic or heterocyclic ring;

R⁷ is (C₁₋₆)alkyl or aryl;

R⁸ and R⁹ independently represent hydrogen, (C₁₋₆)alkyl, aryl or aryl(C₁₋₆)alkyl;

R¹⁰ is hydrogen or (C₁₋₆)alkyl; and

n is 0, 1, 2 or 3;

or a pharmaceutically acceptable salt thereof. (See U.S. Pat. No. 6,372,757.) Examples of such compounds include 1-(4-dimethylaminophenyl)-3-[1,5]naphthyridin-4-yl urea dihydrochloride, 1-(4-methylthiophenyl)-3-[1,5]naphthyridin-4-yl urea hydrochloride, 1-(4-dimethylaminophenyl)-3-[1,5]naphthyridin-4-yl thiourea dihydrochloride, 1-(4-dimethylaminophenyl)-3-[1,6]naphthyridin-4-yl urea, 1-(1-methylindol-5-yl)-3-[1,5]naphthyridin-4-yl urea hydrochloride, 1-(2-methylbenzoxazol-6-yl)-3-[1,5]naphthyridin-4-yl urea hydrochloride, 1-(4-methyl-3,4-dihydro-2H-benzo[1,4]oxazin-7-yl))-3-[1,5]naphthyridin-4-yl urea dihydrochloride, 1-(2-methylbenzoxzol-6-yl)-3-[1,6]naphthyridin-4-yl urea hydrochloride; and 1-(4-dimethylaminophenyl)-3-(6-methoxy-[1,5]naphthyridin-4-yl) urea.

Urea-based OXR1-selective antagonists also include the following compounds: 2-methoxy-5-[3-(2-methyl-[1,5]-naphthyridin-4-yl)ureido]benzoic acid methyl ester, N-cyclopropylmethyl-2-methoxy-5-[3-(2-methyl-[1,5]-naphthyridin-4-yl)ureido]benzamide hydrochloride, 1-(2-methyl-[1,5]-naphthyridin-4-yl)-3-(5-oxo-5,6,7,8-tetrahydronaphthalen-2-yl)urea, 1-(5-hydroxy-5,6,7,8-tetrahydronaphthalen-2-yl)-3-(2-methyl-[1,5]-naphthyridin-4-yl)urea, 1-(3-acetyl-4-methoxyphenyl)-3-(2-methyl-[1,5]-naphthyridin-4-yl)urea, 1-(3-butyryl-4-methoxyphenyl)-3-(2-methyl-[1,5]-naphthyridin-4-yl)urea, 1-(3-acetyl-4-chloro-phenyl)-3-(2-methyl-[1,5]naphthyridin-4-yl)-urea, 1-(3-chloro-4-methoxy-phenyl)-3-(2-methyl-[1,5]naphthyridin-4-yl)-urea, 1-(2-methyl-benzoxazol-6-yl)-3-(2-methyl-[1,5]naphthyridin-4-yl)-urea, methanesulfonic acid 2-chloro-4-[3-(2-methyl-[1,5]naphthyridin-4-yl)-ureido]-phenyl ester, 1-[3-chloro-4-(2-methoxy-ethoxy)-phenyl]-3-(2-methyl-[1,5]naphthyridin-4-yl)-urea, N-ethyl-2-methoxy-5-[3-(2-methyl-[1,5]naphthyridin-4-yl)-ureido]-benzamide, 1-(2-methyl-[1,5]naphthyridin-4-yl)-3-(4-propionyl-phenyl)-urea, 1-[4-(1-hydroxy-1-methyl-ethyl)-phenyl]-3-(2-methyl-[1,5]naphthyridin-4-yl)-urea, 1-(4-acetyl-phenyl)-3-(2-methyl-[1,5]naphthyridin-4-yl)-urea, 1-[4-(1-hydroxy-ethyl)-phenyl]-3-(2-methyl-[1,5]naphthyridin-4-yl)-urea, 1-[4-(1-hydroxy-propyl)-phenyl]-3-(2-methyl-[1,5]naphthyridin-4-yl)-urea, N-butyl-2-methoxy-5-[3-(2-methyl-[1,5]naphthyridin-4-yl)-ureido]-benzamide, 2-methoxy-N-(2-methyl-alkyl)-5-[3-(2-methyl-[1,5]naphthyridin-4-yl)-ureido]-benzamide, 2-methoxy-5-[3-(2-methyl-[1,5]naphthyridin-4-yl)-ureido]-N-thiophen-2-ylmethyl-benzamide, N-cyclopropyl-2-methoxy-5-[3-(2-methyl-[1,5]naphthyridin-4-yl)-ureido]-benzamide, 2-methoxy-5-[3-(2-methyl-[1,5]naphthyridin-4-yl)-ureido]-N-propyl-benzamide, N-benzyl-2-methoxy-5-[3-(2-methyl-[1,5]naphthyridin-4-yl)-ureido]-benzamide, 2-methoxy-N-(2-methoxy-ethyl)-5-[3-(2-methyl-[1,5]naphthyridin-4-yl)-ureido]-benzamide, 2-methoxy-N-(3-methoxy-benzyl)-5-[3-(2-methyl-[1,5]naphthyridin-4-yl)-ureido]-benzamide, 4-[3-(2-methyl-[1,5]naphthyridin-4-yl)-ureido]-benzoic acid ethyl ester, 1-(2-methyl-[1,5]naphthyridin-4-yl)-3-(4-methylsulfanyl-phenyl)-urea, 1-(4-dimethylamino-phenyl)-3-(2-methyl-[1,5]naphthyridin-4-yl)-urea, N-furan-2-ylmethyl-2-methoxy-5-[3-(2-methyl-[1,5]naphthyridin-4-yl)-ureido]-benzamide, 1-(2-cyclopropylmethyl-4-methyl-1-oxo-1,2-dihydro-isoquinolin-7-yl)-3-(2-methyl-[1,5]naphthyridin-4-yl)urea, 1-[3-((E)-3-furan-2-yl-allanoyl)-4-methoxy-phenyl]-3-(2-methyl-[1,5]naphthyridin-4-yl)-urea, or a pharmaceutically acceptable salt thereof. (See U.S. Pat. No. 6,596,730.)

OXR1-selective antagonists useful in the invention include piperdines (U.S. Pat. No. 6,677,354, issued Jan. 13, 2004 to Branch et al. [incorporated by reference herein in its entirety]), such as compounds having the structure: 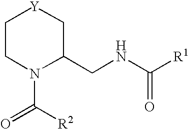
wherein:

Y represents a group (CH₂)ₙ, wherein n represents 0, 1 or 2;

R¹ is phenyl, naphthyl, a mono or bicyclic heteroaryl group containing up to 3 heteroatoms selected from N, O and S; or a group NR³R⁴ wherein one of R³ and R⁴ is hydrogen or optionally substituted (C₁₋₄)alkyl and the other is phenyl, naphthyl or a mono or bicyclic heteroaryl group containing up to 3 heteroatoms selected from N, O and S, or R³ and R⁴ together with the N atom to which they are attached form a 5 to 7-membered cyclic amine which has an optionally fused phenyl ring; any of which R¹ groups may be optionally substituted;

R² represents phenyl or a 5- or 6-membered heteroaryl group containing up to 3 heteroatoms selected from N, O and S, wherein the phenyl or heteroaryl group is substituted by R⁵, and further optional substituents; or R² represents an optionally substituted bicyclic aromatic or bicyclic heteroaromatic group containing up to 3 heteroatoms selected from N, O and S;

R⁵ represents an optionally substituted (C₁₋₄)alkoxy, halo, optionally substituted (C₁₋₆)alkyl, optionally substituted phenyl, or an optionally substituted 5- or 6-membered heterocyclic ring containing up to 3 heteroatoms selected from N, O and S;

or a pharmaceutically acceptable salt thereof. Examples of such compounds include (RS)-2-(benzamidomethyl)-1-((4-(2-methyl-5-phenyl)thiazolyl)carbonyl-piperidine, (RS)-1-((4-(2-methyl-5-phenyl)thiazolyl)carbonyl)-2-((3-phenylureido)methyl)piperidine, (RS)-2-((2-furyl)carbonylaminomethyl)-1-((4-(2-methyl-5-phenyl)thiazolyl) carbonyl)piperidine, (RS)-2-(2-pyridylamidomethyl)-1-((4-(2-methyl-5-phenyl)thiazolyl)carbonyl)piperidine, (RS)-2-((3-((4-fluoro)phenyl)ureido)methyl)-1-((4-(2-methyl-5-phenyl)thiazolyl)carbonyl)piperidine, (RS)-2,3-dihydroindole-1-carboxylic acid (1-(1-(2-(3-methyl-(1,2,4)-oxadiazol-5-yl)-phenyl)-methanoyl)piperidin-2-ylmethyl)amide, (S)-2-(((4-fluoro)phenyl)carbonylaminomethyl)-1-((4-(2-methyl-5-phenyl)thiazolyl)carbonyl)piperidine, (S)-2-((3-((4-fluoro)phenyl)ureido)methyl)-1-((4-(2-methyl-5-phenyl)thiazolyl)carbonyl)piperidine, (S)-2-((7-benzofuranyl)carbonylaminomethyl)-1-((4-(2-methyl-5-(4-fluorophenyl))thiazolyl)carbonyl)piperidine, (S)-2-((4-benzofuranyl)carbonylaminomethyl)-1-((4-(2-methyl-5-(4-fluorophenyl))thiazolyl) carbonyl)piperidine, (S)-2-(((3,4-difluoro)phenyl)carbonylaminomethyl)-1-((4-(2-hydroxymethyl-5-(4-(fluorophenyl))thiazolyl)carbonyl)piperidine, and pharmaceutically acceptable salts thereof.

Exemplary OXR2-selective antagonists include N-acyltetrahydroiso-quinoline derivatives (U.S. Pat. No. 6,838,465, issued Jan. 4, 2005 to Yamada et al. [incorporated by reference herein in its entirety]), such as compounds having the structure: 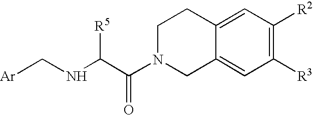
wherein:

R₂ and R₃, each independently, represent lower alkoxy groups;

R₅ represents a benzyl group or a tert-butyl group;

and Ar represents a monocyclic or bicyclic aryl or heteroaryl group optionally having substituent(s) selected from the group consisting of lower alkyl group(s), lower alkoxy group(s), halogen atom(s), halogenated lower alkyl group(s), hydroxyl group(s), carboxyl group(s), lower alkoxy carbonyl group(s), nitro group(s), amino group(s), lower alkylamino group(s), cyano group(s) and methylenedioxy group(s),

or a pharmaceutically acceptable salt thereof.

Additional OXR2-selective antagonists include substituted 4-phenyl-[1,3]-dioxanes (U.S. Pat. No. 6,951,882, issued Oct. 4, 2005 to Carruthers et al. [incorporated by reference herein in its entirety]), such as compounds having the structure: 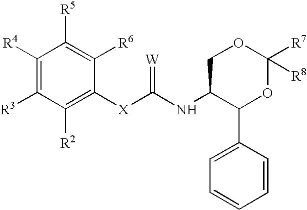
wherein:

R² is H, F, Cl, Br, I, cyano, nitro, COR^{a}, COOR^{a}, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene; wherein R^{a} is H, C₁₋₆ alkyl, C₃₋₇ cycloalkyl, or (C₃₋₇ cycloalkyl)-C₁₋₆ alkylene;

R³ is H, F, Cl, Br, I, cyano, hydroxy, nitro, amino, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene

or R² and R³ taken together with the phenyl ring to which they are attached form a naphthyl;

R⁴ is H, F, Cl, Br, I, cyano, hydroxy, nitro, amino, COR^{b}, COOR^{b}, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene; wherein R^{b} is H, C₁₋₆ alkyl, C₃₋₇ cycloalkyl, or (C₃₋₇ cycloalkyl)-C₁₋₆ alkylene;

R⁵ is H, F, Cl, Br, I, cyano, hydroxy, nitro, amino, C₁₋₆ alkyl, C₁₋₄ alkoxy, C₁₋₆ alkylthio, C₁₋₆ haloalkyl, or C₃₋₇ cycloalkyl;

R⁶ is H, F, Cl, Br, I, cyano, hydroxy, nitro, amino, C₁₋₆ alkyl, C₁₋₆ alkoxy, C₁₋₆alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene;

X is NH, O, or CH₂;

W is S, O, or ═N—CN;

each of R⁷ and R⁸ is independently selected from H, C₁₋₆ alkyl, C₃₋₇ cycloalkyl, (C₃₋₇ cycloalkyl)-C₁₋₆ alkylene, phenyl, and (phenyl)-C₁₋₆ alkylene, provided at least one of R⁷ and R⁸ is not H;

wherein each of the above hydrocarbyl or heterocarbyl moieties can be optionally substituted with between 1 and 3 substituents selected from F, Cl, Br, I, cyano, hydroxy, nitro, amino, COR^{c}, COOR^{c}, C₁₋₃ alkyl, C₁₋₃ alkoxy, C₁₋₃ alkylthio, C₁₋₃ haloalkyl, and C₃₋₆ cycloalkyl; wherein R^{c} is H or C₁₋₆ alkyl;

provided when W is O, X is NH, and R⁷ and R⁸ are each methyl, and R³, R⁴, R⁵, and R⁶ are each H, then R² is not H, 2-chlorophenyl, or 3-quinolinyl;
and pharmaceutically acceptable salts, esters, amides, and hydrates thereof. Additional examples of dioxane-based OXR2-selective antagonists include those having the structure: 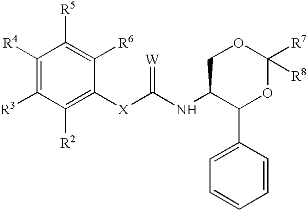
wherein:

R is H, F, Cl, Br, I, cyano, nitro, COR^{a}, COOR^{a}, C₁₋₆ alkyl, C₁₋₆alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene; wherein R^{a} is H, C₁₋₆ alkyl, C₃₋₇ cycloalkyl, or (C₃₋₇ cycloalkyl)-C₁₋₆ alkylene;

R³ is H, F, Cl, Br, I, cyano, hydroxy, nitro, amino, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene

or R² and R³ taken together with the phenyl ring to which they are attached form a naphthyl;

R⁴ is H, F, Cl, Br, I, cyano, hydroxy, nitro, amino, COR^{b}, COOR^{b}, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene; wherein R^{b} is H, C₁₋₆ alkyl, C₃₋₇ cycloalkyl, or (C₃₋₇ cycloalkyl)-C₁₋₆ alkylene;

R⁵ is H, F, Cl, Br, I, cyano, hydroxy, nitro, amino, C₁₋₆ alkyl, C₁₋₆ alkoxy, C₁₋₆ alkylthio, C₁₋₆ haloalkyl, or C₃₋₇ cycloalkyl;

R⁶ is H, F, Cl, Br, I, cyano, hydroxy, nitro, amino, C₁₋₆ alkyl, C₁₋₆ alkoxy, C₁₋₆ alkylthio, C₁₋₆ haloalkyl, C₃₋₇ cycloalkyl, phenyl, C₂₋₉ heterocyclyl, (phenyl)-C₁₋₆ alkylene, (C₂₋₉ heterocyclyl)-C₁₋₆ alkylene, or (C₃₋₇ cycloalkyl)-C₁₋₆ alkylene;

X is NH, O, or CH₂;

W is S, O, or ═N—CN;

each of R⁷ and R⁸ is independently selected from H, C₁₋₆ alkyl, C₃₋₇ cycloalkyl, (C₃₋₇ cycloalkyl)-C₁₋₆ alkylene, phenyl, and (phenyl)-C₁₋₆ alkylene, provided at least one of R⁷ and R⁸ is not H;

wherein each of the above hydrocarbyl or heterocarbyl moieties can be optionally substituted with between 1 and 3 substituents selected from F, Cl, Br, I, cyano, hydroxy, nitro, amino, COR^{c}, COOR^{c}, C₁₋₃ alkyl, C₁₋₃ alkoxy, C₁₋₃ alkylthio, C₁₋₃ haloalkyl, and C₃₋₆ cycloalkyl; wherein R^{c} is H or C₁₋₆ alkyl;

provided when W is O, X is NH, and R⁷ and R⁵ are each methyl, and R³, R⁴, R⁵, and R⁶ are each H, then R² is not H, Br, phenyl, 2-chlorophenyl, or 3-quinolinyl;

provided when W is 0, X is NH, and R⁷ and R⁸ are each methyl, and R⁴, R⁵, and R⁶ are each H, then R³ is not Cl nor is R³ taken together with R²; and

provided when W is O, X is NH, and R⁷ and R⁸ are each methyl, and R², R⁵, and R⁶ are each H, then R⁴ is not Cl;

and pharmaceutically acceptable salts, esters, amides, and hydrates thereof. An exemplary dioxane-based OXR2-selective antagonist is 1-(2,4-dibromo-phenyl)-3-((4S,5S)-2,2-dimethyl-4-phenyl-[1,3]dioxan-5-yl)-urea (McAtee et al (200) Bioorganic & Medicinal Chem Lett 14 4225 [incorporated by reference herein in its entirety]; Johnson & Johnson R&D).

Exemplary OXR2-selective antagonists include N-acyl 6,7-dimethoxy-1,2,3,4-tetrahydroisoquinoline (Hirose et al. (2003) Bioorganic & Medicinal Chem Lett 13 4497 [incorporated by reference herein in its entirety]; Banyu Pharmaceutical Co.).

Orexin A is an example of an agonist that is selective for the OXR1 receptor. [Ala¹¹,D-Leu¹⁵]-orexin B is an OXR2-selective agonist.

3) CRF Modulators.

Examples of direct CRF receptor antagonists useful in the methods of the invention include, but are not limited to, Antisauvagine-30, Astressin ([D-Phe12, Nle21,38, Glu30, Lys33]-CRF (12-41) (see, e.g., Gulyas et al (1995) Proc. Natl. Acad. Sci. USA 92 10575 [incorporated by reference herein in its entirety]), α-helical CRF 9-41 (see, e.g., Swerdlow et al (1989) Neuropsychopharmacology 2 285 [incorporated by reference herein in its entirety]), K 41498 (see, e.g., Ruhmann et al (2002) Peptides 23 453 [incorporated by reference herein in its entirety]), NBI 27914 hydrochloride (5-Chloro-N-(cyclopropylmethyl)-2-methyl-N-propyl-N′-(2,4,6-trichlorophenyl)-4,6-pyrimidinediamine hydrochloride (see, e.g., Chen et al (1996) J. Med. Chem. 39 4358 [incorporated by reference herein in its entirety]), cortagene (see, e.g., Tezval et al. (2004) Proc Natl Acad Sci USA. 101 9468 [incorporated by reference herein in its entirety]), CP-154,526 (see, e.g., Chen, et al. (1997) J. Med. Chem. 40 1749 [incorporated by reference herein in its entirety]); substituted 4-thio-5-oxo-3-pyyrazoline derivatives (Abreu et al., U.S. Pat. No. 5,063,245 [incorporated by reference herein in its entirety]); and substituted 2-aminothiazole derivatives (Courtemanche et al., Australian Patent No. AU-A-41399/93 [incorporated by reference herein in its entirety]). Additional CRF receptor antagonists are described in: Rivier et al., U.S. Pat. No. 4,605,642 (peptide antagonists; [incorporated by reference herein in its entirety]); Rivier et al., Science 224:889, 1984 [incorporated by reference herein in its entirety]; and Haddach et al., U.S. Patent Publication No. 20040266799 (small-molecule antagonists; [incorporated by reference herein in its entirety]).

CRF (6-33) is an exemplary indirect CRF receptor antagonist that acts by displacing CRF from CRF-BP (see e.g., Heinrichs et al (2001) Behav. Brain Res. 122 43 [incorporated by reference herein in its entirety]). Other examples of antagonists that share this mechanism are found in U.S. Pat. No. 5,959,109 (issued Sep. 28, 1999 to Whitten et al. [incorporated by reference herein in its entirety]) and U.S. Pat. No. 6,133,276 (issued Oct. 17, 2000 to Whitten et al [incorporated by reference herein in its entirety].).

U.S. Pat. No. 5,959,109 describes CRF antagonists having the structure: 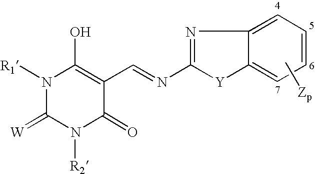
including keto tautomers, stereoisomers and pharmaceutically acceptable acid addition salts thereof, wherein:

W is selected from S and O;

R₁′ and R₂′ are the same or different and independently selected from C₁₋₈ alkyl, C₁₋₈ alkyloxy C₁₋₈ alkyl, aryl, substituted aryl, aryl C₁₋₈ alkyl, substituted aryl C₁₋₈ alkyl, C₃₋₆ cycloalkyl, C₃₋₆ cycloalkyl C₁₋₈ alkyl, C₁₋₁₂ heteroaryl, substituted C₁₋₁₂ heteroaryl, C₁₋₁₂ heteroaryl C₁₋₈ alkyl, substituted C₁₋₁₂ heteroaryl C₁₋₈ alkyl, C₁₋₁₂ heteroaryl C₂₋₈ alkenyl and substituted C₁₋₁₂ heteroaryl C₂₋₈ alkenyl;

Y is selected from NH, S, O and N(CH₃); and

Z is a substituent, p is 0, 1, 2 or 3 and represents the number of Z substituents, and each occurrence of Z is independently selected from halo, nitro, C₁₋₈ alkyloxy, C₁₋₈ alkyl, C₂₋₈ alkenyl, C₂₋₈ alkynyl and C₁₋₈ haloalkyl.

U.S. Pat. No. 6,133,276 describes CRF antagonists having the structure: 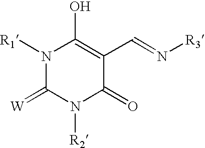
including keto tautomers, stereoisomers and pharmaceutically acceptable acid addition salts thereof, wherein

W is selected from S and O;

R₁′ and R₂′ are the same or different and independently selected from C₁₋₈ alkyl, C₁₋₈ alkyloxyC₁₋₈ alkyl, aryl, substituted aryl, arylC₁₋₈ alkyl, substituted arylC₁₋₈ alkyl, C₃₋₆ cycloalkyl, C₃₋₆ cycloalkylC₁₋₈ alkyl, C₁₋₁₂ heteroaryl, substituted C₁₋₁₂ heteroaryl, C₁₋₁₂ heteroarylC₁₋₈ alkyl, substituted C₁₋₁₂ heteroarylC₁₋₈ alkyl, C₁₋₁₂ heteroarylC₂₋₈ alkenyl and substituted C₁₋₁₂ heteroarylC₂₋₈ alkenyl; and

R₃′ is selected from C₁₋₁₂ heteroaryl, substituted C₁₋₁₂ heteroaryl, C₁₋₁₂ heteroarylC₁₋₈ alkyl, substituted C₁₋₁₂ heteroarylC₁₋₈ alkyl, C₁₋₁₂ heteroarylC₂₋₈ alkenyl, substituted C₁₋₁₂ heteroarylC₂₋₈ alkenyl and the following structures:

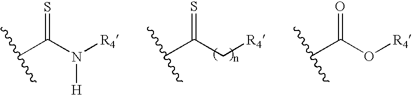

wherein n is 0, 1 or 2; and

R₄′ is selected from aryl and substituted aryl with one or more substituents independently selected from halo and C₁₋₈ alkyloxy.

Examples of direct CRF receptor agonists include, but are not limited to, CRF (see, e.g., Tache et al (1983) Science 222 935 [incorporated by reference herein in its entirety]), Sauvagine (see, e.g., Montecucchi and Henschen (1981) Int. J. Pept. Protein Res. 18 113 [incorporated by reference herein in its entirety]), Stressin I (see, e.g., Rivier et al (2001) 413-17 [incorporated by reference herein in its entirety]), and Urocortin (see, e.g., Perrin and Vale (1999) Ann. N.Y. Acad. Sci. 885 312 [incorporated by reference herein in its entirety]).

The CRF agonist/antagonist can be non-selective or selective for a particular receptor subtype (e.g., CRF1 or CRF2). In particular embodiments, the CRF receptor agonist or antagonist is selective for CRF2. Exemplary CRF2-selective antagonists include Antisauvagine-30 and K 41498. Most of the above-described CRF agonists/antagonists are available from KOMA Biotech Inc., Seoul, Korea (http://www.komabiotech.com).

E) Administration of Orexin and CRF Modulators.

The mode of administration of the orexin, orexin receptor, CRF, CRF-BP, and/or CRF receptor modulators depends on the nature of the particular agent. Antisense molecules, catalytic RNAs (ribozymes), catalytic DNAs, small organic molecules, and other molecules (e.g. lipids, antibodies, etc.) used as orexin, orexin receptor, CRF, CRF-BP, and/or CRF receptor antagonists can be formulated as pharmaceuticals (e.g. with suitable excipient) and delivered using standard pharmaceutical formulation and delivery methods as described below. Antisense molecules, catalytic RNAs (ribozymes), catalytic DNAs, and additionally, knockout constructs, and constructs encoding intrabodies can be delivered and (if necessary) expressed in target cells (e.g. vascular endothelial cells) using methods of gene therapy, e.g. as described below.

In order to carry out the methods of the invention, one or more inhibitors or enhancers of orexin, orexin receptor, CRF, CRF-BP, and/or CRF receptor expression and/or activity and/or interaction (e.g. ribozymes, antibodies, antisense molecules, small organic molecules, etc.) are administered to an individual to modulate NMDA receptor potentiation (e.g. to modulate a behavioral response to the consumption of alcohol and/or other substances of abuse). While this invention is described generally with reference to human subjects, veterinary applications are contemplated within the scope of this invention.

Various modulators may be administered, if desired, in the form of salts, esters, amides, prodrugs, derivatives, and the like, provided the salt, ester, amide, prodrug or derivative is suitable pharmacologically, i.e., effective in the present method. Salts, esters, amides, prodrugs and other derivatives of the active agents may be prepared using standard procedures known to those skilled in the art of synthetic organic chemistry and described, for example, by March (1992) Advanced Organic Chemistry; Reactions, Mechanisms and Structure, 4th Ed. N.Y. Wiley-Interscience.

F) Formulations.

The active agents and various derivatives and/or formulations thereof are useful for parenteral, topical, oral, or local administration, such as by aerosol or transdermally, for prophylactic and/or therapeutic treatment of substance abuse or any of the other conditions listed above. The pharmaceutical compositions can be administered in a variety of unit dosage forms depending upon the method of administration. Suitable unit dosage forms, include, but are not limited to powders, tablets, pills, capsules, lozenges, suppositories, etc.

The active agent(s) and various derivatives and/or formulations thereof are typically combined with a pharmaceutically acceptable carrier (excipient) to form a pharmacological composition. Pharmaceutically acceptable carriers can contain one or more physiologically acceptable compound(s) that act, for example, to stabilize the composition or to increase or decrease the absorption of the active agent(s). Physiologically acceptable compounds can include, for example, carbohydrates, such as glucose, sucrose, or dextrans, antioxidants, such as ascorbic acid or glutathione, chelating agents, low molecular weight proteins, compositions that reduce the clearance or hydrolysis of the active agents, or excipients or other stabilizers and/or buffers.

Other physiologically acceptable compounds include wetting agents, emulsifying agents, dispersing agents or preservatives which are particularly useful for preventing the growth or action of microorganisms. Various preservatives are well known and include, for example, phenol and ascorbic acid. One skilled in the art would appreciate that the choice of pharmaceutically acceptable carrier(s), including a physiologically acceptable compound depends, for example, on the route of administration of the active agent(s) and on the particular physio-chemical characteristics of the active agent(s). The excipients are preferably sterile and generally free of undesirable matter. These compositions may be sterilized by conventional, well known sterilization techniques.

The concentration of active agent(s) in the formulation can vary widely, and will be selected primarily based on fluid volumes, viscosities, body weight and the like in accordance with the particular mode of administration selected and the patient's needs.

In therapeutic applications, the compositions of this invention are administered to a patient suffering from a condition in an amount sufficient to cure or at least partially arrest the condition and/or mitigate its symptoms (e.g. to reduce relapse to drug abuse, etc.) An amount adequate to accomplish this is defined as a “therapeutically effective dose.” Amounts effective for this use will depend upon the severity of the disease and the general state of the patient's health. Single or multiple administrations of the compositions may be administered depending on the dosage and frequency as required and tolerated by the patient. In any event, the composition should provide a sufficient quantity of the active agents of the formulations of this invention to effectively treat (ameliorate one or more symptoms) the patient.

In certain preferred embodiments, the orexin, orexin receptor, CRF, CRF-BP, and/or CRF receptor modulators are administered orally (e.g. via a tablet) or as an injectable in accordance with standard methods well known to those of skill in the art. In other preferred embodiments, the orexin, orexin receptor, CRF, CRF-BP, and/or CRF receptor modulators can also be delivered through the skin using conventional transdermal drug delivery systems, i.e., transdermal “patches” wherein the active agent(s) are typically contained within a laminated structure that serves as a drug delivery device to be affixed to the skin. In such a structure, the drug composition is typically contained in a layer, or “reservoir,” underlying an upper backing layer. It will be appreciated that the term “reservoir” in this context refers to a quantity of “active ingredient(s)” that is ultimately available for delivery to the surface of the skin. Thus, for example, the “reservoir” may include the active ingredient(s) in an adhesive on a backing layer of the patch, or in any of a variety of different matrix formulations known to those of skill in the art. The patch may contain a single reservoir, or it may contain multiple reservoirs.

In one embodiment, the reservoir comprises a polymeric matrix of a pharmaceutically acceptable contact adhesive material that serves to affix the system to the skin during drug delivery. Examples of suitable skin contact adhesive materials include, but are not limited to, polyethylenes, polysiloxanes, polyisobutylenes, polyacrylates, polyurethanes, and the like. Alternatively, the drug-containing reservoir and skin contact adhesive are present as separate and distinct layers, with the adhesive underlying the reservoir which, in this case, may be either a polymeric matrix as described above, or it may be a liquid or hydrogel reservoir, or may take some other form. The backing layer in these laminates, which serves as the upper surface of the device, preferably functions as a primary structural element of the “patch” and provides the device with much of its flexibility. The material selected for the backing layer is preferably substantially impermeable to the active agent(s) and any other materials that are present.

The foregoing formulations and administration methods are intended to be illustrative and not limiting. It will be appreciated that, using the teaching provided herein, other suitable formulations and modes of administration can be readily devised.

II. Assays for Agents that Modulate Orexin and/or CRF Potentiation of NMDA Currents.

As indicated above, in one aspect, this invention pertains to the discovery of mechanisms underlying orexin and/or CRF potentiation of NMDA-mediated currents. These effects are mediated via the interaction of orexin and the orexin receptor (e.g., OXR1) and the interaction of CRF, CRF binding protein (CRF-BP), and the CRF receptor (e.g., CRF2). Thus, agents that modulate the interaction of orexin and/or the orexin receptor and/or that modulate (e.g., upregulate and/or downregulate) the expression and/or activity of orexin and/or the orexin receptor are expected to have prophylactic and/or therapeutic utility as described herein. Similarly, agents that modulate the interaction of CRF and/or CRF-BP and/or the CRF receptor (e.g., CRF2R) and/or that modulate (e.g., upregulate and/or downregulate) the expression and/or activity of CRF and/or CRF-BP and/or the CRF receptor are expected to have prophylactic and/or therapeutic utility, alone or in combination with agents acting on the orexin pathway. In certain embodiments this invention provides methods of prescreening and screening for agents that modulate the interaction, activity, and/or expression of orexin, the orexin receptor, CRF and/or CRF-BP and/or the CRF receptor. The prescreening and screening methods are described herein with respect to CRF and/or CRF-BP and/or the CRF receptor for ease of discussion; however, those skilled in the art readily appreciate that assays employing CRF are equally applicable to orexin and that assays employing the CRF receptor are equally applicable to the orexin receptor. Thus, for example, references to the use or detection of CRF2 receptor in the assays described below are understood as applying equally to the OXR1 receptor.

The methods typically involve direct assays for the interaction of CRF and/or CRF-BP and/or the CRF2 receptor or detecting the activity of CRF2 receptor or potentiation of NMDA receptors and/or detecting alterations in the expression level and/or activity level of CRF, CRF-BP, and/or CRF2 receptor genes or gene products caused by the treatment with one or more of the agent(s) in question. An elevated expression level or activity level produced by the agent as, e.g., compared to a negative control where the test agent is absent or at reduced concentration indicates that the agent upregulates activity or expression of the factor(s) in question. Conversely, decreased expression level or activity level resulting from treatment with the agent as compared to a negative control where the test agent is absent or at reduced concentration indicates that the agent down-regulates expression or activity of the factor(s).

A) Assaying for Modulators of Protein Interaction.

In certain embodiments, this invention pertains to assays for agents that modulate the interaction of CRF and/or CRF-BP and/or CRF2 receptor and thereby agonize or antagonize CRF activity at the CRF2 receptor. In certain embodiments, this involves contacting a cell, tissue, or organism with one or more test agents and evaluating the effect of the test agent(s) on the interaction of CRF, CRF-BP, and/or CRF2 receptor. Methods of screening for the effect of test agents on protein/protein interactions are well known to those of skill in the art. Such methods include, but are not limited to two-hybrid systems, gel-shift assays, and the like.

In a two-hybrid system, two chimeric molecules are created, one of which bears a nucleic acid binding region, the other of which bears an expression control element (e.g. a transactivation or repressor domain). The molecules each further comprise one of the two proteins whose interaction is to be assayed. The chimeric molecule comprising the DNA binding domain binds to a “substrate” nucleic acid. When the two proteins of interest interact/bind, i.e., the domain of the chimeric molecule recognizes and binds to its cognate binding partner on the second chimeric molecule thereby recruiting that molecule to the nucleic acid whereby the expression control element alters (e.g. activates) expression of a gene or cDNA comprising the underlying nucleic acid. This provides a detectable signal that is an indicator of protein/protein interaction. The effect of one or more test agent(s) on this interaction can then readily be evaluated. Two-hybrid systems are well known to those of skill in the art (see, e.g., Fields and Song (1989) Nature 340: 245-246).

In a gel-shift assay, one or more of the proteins whose binding is to be evaluated is labeled with a detectable label. Where the proteins bind to each other, the mobility of the complex thus formed is different than the mobility of the individual component proteins and can readily be detected (e.g. in an electrophoretic gel). The effect of one or more test agents on the formation of such complexes can then readily be detected.

These assays are intended to be illustrative and not limiting. Using the teaching provided herein, numerous other assays for evaluating the effect of one or more test agents on CRF, CRF-BP and CRF2R interaction can readily be provided.

B) Assaying for Modulators of Activity.

In one embodiment, the effect of one or more test agents on CRF-BP, CRF and/or CRF2 receptor activity can be directly evaluated. In one such approach, the test agent(s) are contacted to a neurological tissue preparation (e.g. a brain slice preparation) and the effect of the test agent on CRF potentiation of NMDA receptor currents is evaluated using electrophysiological techniques as described herein in Example 1.

C) Assaying for Modulators of Gene Expression.

Expression levels of a gene can be altered by changes in by the transcription of the gene product (i.e. transcription of mRNA), and/or by changes in translation of the gene product (i.e. translation of the protein), and/or by post-translational modification(s) (e.g. protein folding, glycosylation, etc.). Thus, preferred assays of this invention typically entail contacting a test cell, tissue, or animal with one or more test agents, and assaying for level of transcribed mRNA, level of translated protein, activity of translated protein, etc. Examples of such approaches are described below.

1) Nucleic-Acid Based Assays.

a. Target Molecules.

Changes in expression level can be detected by measuring changes in mRNA and/or a nucleic acid derived from the mRNA (e.g. reverse-transcribed cDNA, etc.). In order to measure the CRF, and/or CRF-BP, and/or CRF2 receptor expression level, it is desirable to provide a nucleic acid sample for such analysis. In preferred embodiments, the nucleic acid is found in or derived from a biological sample. The term “biological sample,” as used herein, refers to a sample obtained from an organism or from components (e.g., cells) of an organism. The sample may be of any biological tissue or fluid. Biological samples may also include organs or sections of tissues such as frozen sections taken for histological purposes.

The nucleic acid (e.g., mRNA or nucleic acid derived from mRNA) is, in certain preferred embodiments, isolated from the sample according to any of a number of methods well known to those of skill in the art. Methods of isolating mRNA are well known to those of skill in the art. For example, methods of isolation and purification of nucleic acids are described in detail in by Tijssen ed., (1993) Chapter 3 of Laboratory Techniques in Biochemistry and Molecular Biology: Hybridization With Nucleic Acid Probes, Part I. Theory and Nucleic Acid Preparation, Elsevier, N.Y. and Tijssen ed.

In a preferred embodiment, the “total” nucleic acid is isolated from a given sample using, for example, an acid guanidinium-phenol-chloroform extraction method, and polyA+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)).

Frequently, it is desirable to amplify the nucleic acid sample prior to assaying for expression level. Methods of amplifying nucleic acids are well known to those of skill in the art and include, but are not limited to, polymerase chain reaction (PCR, see, e.g., Innis, et al., (1990) PCR Protocols. A guide to Methods and Application. Academic Press, Inc. San Diego) ligase chain reaction (LCR) (see Wu and Wallace (1989) Genomics 4: 560, Landegren et al. (1988) Science 241: 1077, and Barringer et al. (1990) Gene 89: 117, transcription amplification (Kwoh et al. (1989) Proc. Natl. Acad. Sci. USA 86: 1173), self-sustained sequence replication (Guatelli et al. (1990) Proc. Nat. Acad. Sci. USA 87: 1874), dot PCR, and linker adapter PCR, etc.).

In a particularly preferred embodiment, where it is desired to quantify the transcription level (and thereby expression) of factor(s) of interest in a sample, the nucleic acid sample is one in which the concentration of the mRNA transcript(s), or the concentration of the nucleic acids derived from the transcript(s), is proportional to the transcription level (and therefore expression level) of that gene. Similarly, in hybridization-based assays, it is preferred that the hybridization signal intensity be proportional to the amount of hybridized nucleic acid. While it is preferred that the proportionality be relatively strict (e.g., a doubling in transcription rate results in a doubling in mRNA transcript in the sample nucleic acid pool and a doubling in hybridization signal), one of skill will appreciate that the proportionality can be more relaxed and even non-linear. Thus, for example, an assay where a 5-fold difference in concentration of the target mRNA results in a 3- to 6-fold difference in hybridization intensity is sufficient for most purposes.

Where more precise quantification is required, appropriate controls can be run to correct for variations introduced in sample preparation and hybridization as described herein. In addition, serial dilutions of “standard” target nucleic acids (e.g., mRNAs) can be used to prepare calibration curves according to methods well known to those of skill in the art. Of course, where simple detection of the presence or absence of a transcript or of large differences of changes in nucleic acid concentration is desired, no elaborate control or calibration is required.

In the simplest embodiment, the nucleic acid sample is the total mRNA or a total cDNA isolated and/or otherwise derived from a biological sample (e.g. a sample from a neural cell or tissue). The nucleic acid may be isolated from the sample according to any of a number of methods well known to those of skill in the art as indicated above.

b. Hybridization-Based Assays.

Using the known sequence of CRF, and/or CRF-BP, and/or CRF2 receptor, detecting and/or quantifying the transcript(s) can be routinely accomplished using nucleic acid hybridization techniques (see, e.g., Sambrook et al. supra). For example, one method for evaluating the presence, absence, or quantity of reverse-transcribed cDNA involves a “Southern Blot”. In a Southern Blot, the DNA (e.g., reverse-transcribed CRF, and/or CRF-BP, and/or CRF2 receptor mRNA), typically fragmented and separated on an electrophoretic gel, is hybridized to a probe specific for the nucleic acid encoding the CRF, and/or CRF-BP, and/or CRF2 receptor. Comparison of the intensity of the hybridization signal from the target-specific probe with a “control” probe (e.g. a probe for a “housekeeping gene”) provides an estimate of the relative expression level of the target nucleic acid.

Alternatively, the CRF, and/or CRF-BP, and/or CRF2 receptor mRNA can be directly quantified in a Northern blot. In brief, the mRNA is isolated from a given cell sample using, for example, an acid guanidinium-phenol-chloroform extraction method. The mRNA is then electrophoresed to separate the mRNA species and the mRNA is transferred from the gel to a nitrocellulose membrane. As with the Southern blots, labeled probes are used to identify and/or quantify the target mRNA. Appropriate controls (e.g. probes to housekeeping genes) provide a reference for evaluating relative expression level.

An alternative means for determining the CRF, and/or CRF-BP, and/or CRF2 receptor expression level is in situ hybridization. In situ hybridization assays are well known (e.g., Angerer (1987) Meth. Enzymol 152: 649). Generally, in situ hybridization comprises the following major steps: (1) fixation of tissue or biological structure to be analyzed; (2) prehybridization treatment of the biological structure to increase accessibility of target DNA and to reduce nonspecific binding; (3) hybridization of the mixture of nucleic acids to the nucleic acid in the biological structure or tissue; (4) post-hybridization washes to remove nucleic acid fragments not bound in the hybridization; and (5) detection of the hybridized nucleic acid fragments. The reagent used in each of these steps and the conditions for use vary depending on the particular application.

In some applications it is necessary to block the hybridization capacity of repetitive sequences. Thus, in some embodiments, tRNA, human genomic DNA, or Cot-1 DNA is used to block non-specific hybridization.

c. Amplification-Based Assays.

In another embodiment, amplification-based assays can be used to measure CRF, and/or CRF-BP, and/or CRF2 receptor expression (transcription) level. In such amplification-based assays, the target nucleic acid sequences (i.e., CRF, and/or CRF-BP, and/or CRF2 receptor nucleic acid(s)) act as template(s) in amplification reaction(s) (e.g. Polymerase Chain Reaction (PCR) or reverse-transcription PCR (RT-PCR)). In a quantitative amplification, the amount of amplification product will be proportional to the amount of template in the original sample. Comparison to appropriate (e.g. healthy tissue or cells unexposed to the test agent) controls provides a measure of the CRF, and/or CRF-BP, and/or CRF2 receptor transcript level.

Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. Detailed protocols for quantitative PCR are provided in Innis et al. (1990) PCR Protocols, A Guide to Methods and Applications, Academic Press, Inc. N.Y.). One approach, for example, involves simultaneously co-amplifying a known quantity of a control sequence using the same primers as those used to amplify the target. This provides an internal standard that may be used to calibrate the PCR reaction.

One preferred internal standard is a synthetic AW106 cRNA. The AW106 cRNA is combined with RNA isolated from the sample according to standard techniques known to those of skill in the art. The RNA is then reverse transcribed using a reverse transcriptase to provide copy DNA. The cDNA sequences are then amplified (e.g., by PCR) using labeled primers. The amplification products are separated, typically by electrophoresis, and the amount of labeled nucleic acid (proportional to the amount of amplified product) is determined. The amount of mRNA in the sample is then calculated by comparison with the signal produced by the known AW106 RNA standard. Detailed protocols for quantitative PCR are provided in PCR Protocols, A Guide to Methods and Applications, Innis et al. (1990) Academic Press, Inc. N.Y. The known nucleic acid sequence(s) for CRF, and/or CRF-BP, and/or CRF2 receptor are sufficient to enable one of skill to routinely select primers to amplify any portion of the gene.

d. Hybridization Formats and Optimization of Hybridization.

i. Array-Based Hybridization Formats.

In one embodiment, the methods of this invention can be utilized in array-based hybridization formats. Arrays are a multiplicity of different “probe” or “target” nucleic acids (or other compounds) attached to one or more surfaces (e.g., solid, membrane, or gel). In a preferred embodiment, the multiplicity of nucleic acids (or other moieties) is attached to a single contiguous surface or to a multiplicity of surfaces juxtaposed to each other.

In an array format a large number of different hybridization reactions can be run essentially “in parallel.” This provides rapid, essentially simultaneous, evaluation of a number of hybridizations in a single “experiment.” Methods of performing hybridization reactions in array-based formats are well known to those of skill in the art (see, e.g., Pastinen (1997) Genome Res. 7: 606-614; Jackson (1996) Nature Biotechnology 14:1685; Chee (1995) Science 274: 610; WO 96/17958, Pinkel et al. (1998) Nature Genetics 20: 207-211).

Arrays, particularly nucleic acid arrays can be produced according to a wide variety of methods well known to those of skill in the art. For example, in a simple embodiment, “low density” arrays can simply be produced by spotting (e.g. by hand using a pipette) different nucleic acids at different locations on a solid support (e.g. a glass surface, a membrane, etc.).

This simple spotting, approach has been automated to produce high density spotted arrays (see, e.g., U.S. Pat. No. 5,807,522). This patent describes the use of an automated system that taps a microcapillary against a surface to deposit a small volume of a biological sample. The process is repeated to generate high density arrays.

Arrays can also be produced using oligonucleotide synthesis technology. Thus, for example, U.S. Pat. No. 5,143,854 and PCT Patent Publication Nos. WO 90/15070 and 92/10092 teach the use of light-directed combinatorial synthesis of high density oligonucleotide arrays. Synthesis of high density arrays is also described in U.S. Pat. Nos. 5,744,305, 5,800,992 and 5,445,934.

ii. Other Hybridization Formats.

As indicated above, a variety of nucleic acid hybridization formats are known to those skilled in the art. For example, common formats include sandwich assays and competition or displacement assays. Such assay formats are generally described in Hames and Higgins (1985) Nucleic Acid Hybridization, A Practical Approach, IRL Press; Gall and Pardue (1969) Proc. Natl. Acad. Sci. USA 63: 378-383; and John et al. (1969) Nature 223: 582-587.

Sandwich assays are commercially useful hybridization assays for detecting or isolating nucleic acid sequences. Such assays utilize a “capture” nucleic acid covalently immobilized to a solid support and a labeled “signal” nucleic acid in solution. The sample will provide the target nucleic acid. The capture nucleic acid and signal nucleic acid probe hybridize with the target nucleic acid to form a “sandwich” hybridization complex. To be most effective, the signal nucleic acid should not hybridize with the capture nucleic acid.

Typically, labeled signal nucleic acids are used to detect hybridization. Complementary nucleic acids or signal nucleic acids may be labeled by any one of several methods typically used to detect the presence of hybridized polynucleotides. The most common method of detection is the use of autoradiography with ³H, ¹²⁵I, ³⁵S, ¹⁴C, or ³²P-labelled probes or the like. Other labels include ligands that bind to labeled antibodies, fluorophores, chemi-luminescent agents, enzymes, and antibodies which can serve as specific binding pair members for a labeled ligand.

Detection of a hybridization complex may require the binding of a signal-generating complex to a duplex of target and probe polynucleotides or nucleic acids. Typically, such binding occurs through ligand and anti-ligand interactions as between a ligand-conjugated probe and an anti-ligand conjugated with a signal.

The sensitivity of the hybridization assays may be enhanced through use of a nucleic acid amplification system that multiplies the target nucleic acid being detected. Examples of such systems include the polymerase chain reaction (PCR) system and the ligase chain reaction (LCR) system. Other methods recently described in the art are the nucleic acid sequence-based amplification (NASBAO, Cangene, Mississauga, Ontario) and Q Beta Replicase systems.

e. Optimization of Hybridization Conditions.

Nucleic acid hybridization simply involves providing a denatured probe and target nucleic acid under conditions where the probe and its complementary target can form stable hybrid duplexes through complementary base pairing. The nucleic acids that do not form hybrid duplexes are then washed away leaving the hybridized nucleic acids to be detected, typically through detection of an attached detectable label. It is generally recognized that nucleic acids are denatured by increasing the temperature or decreasing the salt concentration of the buffer containing the nucleic acids, adding chemical agents, or the raising of the pH. Under low stringency conditions (e.g., low temperature and/or high salt and/or high target concentration) hybrid duplexes (e.g., DNA:DNA, RNA:RNA, or RNA:DNA) will form even where the annealed sequences are not perfectly complementary. Thus, specificity of hybridization is reduced at lower stringency. Conversely, at higher stringency (e.g., higher temperature or lower salt) successful hybridization requires fewer mismatches.

One of skill in the art will appreciate that hybridization conditions may be selected to provide any degree of stringency. In a preferred embodiment, hybridization is performed at low stringency to ensure hybridization and then subsequent washes are performed at higher stringency to eliminate mismatched hybrid duplexes. Successive washes may be performed at increasingly higher stringency (e.g., down to as low as 0.25×SSPE at 37° C. to 70° C.) until a desired level of hybridization specificity is obtained. Stringency can also be increased by addition of agents such as formamide. Hybridization specificity may be evaluated by comparison of hybridization to the test probes with hybridization to the various controls that can be present.

In general, there is a tradeoff between hybridization specificity (stringency) and signal intensity. Thus, in a preferred embodiment, the wash is performed at the highest stringency that produces consistent results and that provides a signal intensity greater than approximately 10% of the background intensity. The hybridized array may be washed at successively higher stringency solutions and read between each wash. Analysis of the data sets thus produced will reveal a wash stringency above which the hybridization pattern is not appreciably altered and which provides adequate signal for the particular probes of interest.

In a preferred embodiment, background signal is reduced by the use of a blocking reagent (e.g., tRNA, sperm DNA, cot-1 DNA, etc.) during the hybridization to reduce non-specific binding. The use of blocking agents in hybridization is well known to those of skill in the art (see, e.g., Chapter 8 in P. Tijssen, supra.)

Methods of optimizing hybridization conditions are well known to those of skill in the art (see, e.g., Tijssen (1993) Laboratory Techniques in Biochemistry and Molecular Biology, Vol. 24: Hybridization With Nucleic Acid Probes, Elsevier, N.Y.).

Optimal conditions are also a function of the sensitivity of label (e.g., fluorescence) detection for different combinations of substrate type, fluorochrome, excitation and emission bands, spot size and the like. Low fluorescence background surfaces can be used (see, e.g., Chu (1992) Electrophoresis 13:105-114). The sensitivity for detection of spots (“target elements”) of various diameters on the candidate surfaces can be readily determined by, e.g., spotting a dilution series of fluorescently end labeled DNA fragments. These spots are then imaged using conventional fluorescence microscopy. The sensitivity, linearity, and dynamic range achievable from the various combinations of fluorochrome and solid surfaces (e.g., glass, fused silica, etc.) can thus be determined. Serial dilutions of pairs of fluorochromes in known relative proportions can also be analyzed. This determines the accuracy with which fluorescence ratio measurements reflect actual fluorochrome ratios over the dynamic range permitted by the detectors and fluorescence of the substrate upon which the probe has been fixed.

f. Labeling and Detection of Nucleic Acids.

The probes used herein for detection of CRF, and/or CRF-BP, and/or CRF2 receptor expression levels can be full-length or less than the full length of the CRF, and/or CRF-BP, and/or CRF2 receptor mRNA(s). Shorter probes are generally empirically tested for specificity. Preferred probes are sufficiently long so as to specifically hybridize with the target nucleic acid(s) under stringent conditions. The preferred size range is from about 20 bases to the length of CRF, and/or CRF-BP, and/or CRF2 receptor mRNA, more preferably from about 30 bases to the length of the CRF, and/or CRF-BP, and/or CRF2 receptor mRNA, and most preferably from about 40 bases to the length of CRF, and/or CRF-BP, and/or CRF2 receptor mRNA.

The probes are typically labeled, with a detectable label. Detectable labels suitable for use in the present invention include any composition detectable by spectroscopic, photochemical, biochemical, immunochemical, electrical, optical or chemical means. Useful labels in the present invention include biotin for staining with labeled streptavidin conjugate, magnetic beads (e.g., Dynabeads™), fluorescent dyes (e.g., fluorescein, texas red, rhodamine, green fluorescent protein, and the like, see, e.g., Molecular Probes, Eugene, Oreg., USA), radiolabels (e.g., ³H, ¹²⁵I, ³⁵S, ¹⁴C, or ³²P), enzymes (e.g., horse radish peroxidase, alkaline phosphatase and others commonly used in an ELISA), and colorimetric labels such as colloidal gold (e.g., gold particles in the 40-80 nm diameter size range scatter green light with high efficiency) or colored glass or plastic (e.g., polystyrene, polypropylene, latex, etc.) beads. Patents teaching the use of such labels include U.S. Pat. Nos. 3,817,837; 3,850,752; 3,939,350; 3,996,345; 4,277,437; 4,275,149; and 4,366,241.

A fluorescent label is preferred because it provides a very strong signal with low background. It is also optically detectable at high resolution and sensitivity through a quick scanning procedure. The nucleic acid samples can all be labeled with a single label, e.g., a single fluorescent label. Alternatively, in another embodiment, different nucleic acid samples can be simultaneously hybridized where each nucleic acid sample has a different label. For instance, one target could have a green fluorescent label and a second target could have a red fluorescent label. The scanning step will distinguish sites of binding of the red label from those binding the green fluorescent label. Each nucleic acid sample (target nucleic acid) can be analyzed independently from one another.

Suitable chromogens which can be employed include those molecules and compounds which absorb light in a distinctive range of wavelengths so that a color can be observed or, alternatively, which emit light when irradiated with radiation of a particular wave length or wave length range, e.g., fluorescers.

Desirably, fluorescent labels should absorb light above about 300 nm, preferably about 350 nm, and more preferably above about 400 nm, usually emitting at wavelengths greater than about 10 nm higher than the wavelength of the light absorbed. It should be noted that the absorption and emission characteristics of the bound dye can differ from the unbound dye. Therefore, when referring to the various wavelength ranges and characteristics of the dyes, it is intended to indicate the dyes as employed and not the dye which is unconjugated and characterized in an arbitrary solvent.

Detectable signal can also be provided by chemiluminescent and bioluminescent sources. Chemiluminescent sources include a compound which becomes electronically excited by a chemical reaction and can then emit light which serves as the detectable signal or donates energy to a fluorescent acceptor. Alternatively, luciferins can be used in conjunction with luciferase or lucigenins to provide bioluminescence.

Spin labels are provided by reporter molecules with an unpaired electron spin which can be detected by electron spin resonance (ESR) spectroscopy. Exemplary spin labels include organic free radicals, transitional metal complexes, particularly vanadium, copper, iron, and manganese, and the like. Exemplary spin labels include nitroxide free radicals.

The label can be added to the target (sample) nucleic acid(s) prior to, or after the hybridization. So called “direct labels” are detectable labels that are directly attached to or incorporated into the target (sample) nucleic acid prior to hybridization. In contrast, so called “indirect labels” are joined to the hybrid duplex after hybridization. Often, the indirect label is attached to a binding moiety that has been attached to the target nucleic acid prior to the hybridization. Thus, for example, the target nucleic acid may be biotinylated before the hybridization. After hybridization, an avidin-conjugated fluorophore will bind the biotin bearing hybrid duplexes providing a label that is easily detected. For a detailed review of methods of labeling nucleic acids and detecting labeled hybridized nucleic acids see Laboratory Techniques in Biochemistry and Molecular Biology, Vol. 24: Hybridization With Nucleic Acid Probes, P. Tijssen, ed. Elsevier, N.Y., (1993)).

Fluorescent labels are easily added during an in vitro transcription reaction. Thus, for example, fluorescein labeled UTP and CTP can be incorporated into the RNA produced in an in vitro transcription.

The labels can be attached directly or through a linker moiety. In general, the site of label or linker-label attachment is not limited to any specific position. For example, a label may be attached to a nucleoside, nucleotide, or analogue thereof at any position that does not interfere with detection or hybridization as desired. For example, certain Label-ON Reagents from Clontech (Palo Alto, Calif.) provide for labeling interspersed throughout the phosphate backbone of an oligonucleotide and for terminal labeling at the 3′ and 5′ ends. As shown for example herein, labels can be attached at positions on the ribose ring or the ribose can be modified and even eliminated as desired. The base moieties of useful labeling reagents can include those that are naturally occurring or modified in a manner that does not interfere with the purpose to which they are put. Modified bases include but are not limited to 7-deaza A and G, 7-deaza-8-aza A and G, and other heterocyclic moieties.

It will be recognized that fluorescent labels are not to be limited to single species organic molecules, but include inorganic molecules, multi-molecular mixtures of organic and/or inorganic molecules, crystals, heteropolymers, and the like. Thus, for example, CdSe—CdS core-shell nanocrystals enclosed in a silica shell can be easily derivatized for coupling to a biological molecule (Bruchez et al. (1998) Science, 281: 2013-2016). Similarly, highly fluorescent quantum dots (zinc sulfide-capped cadmium selenide) have been covalently coupled to biomolecules for use in ultrasensitive biological detection (Warren and Nie (1998) Science, 281: 2016-2018).

2) Polypeptide-Based Assays.

The CRF, and/or CRF-BP, and/or CRF2 receptor polypeptide(s) can be detected and quantified by any of a number of methods well known to those of skill in the art. These may include analytic biochemical methods such as electrophoresis, capillary electrophoresis, high performance liquid chromatography (HPLC), thin layer chromatography (TLC), hyperdiffusion chromatography, and the like, or various immunological methods such as fluid or gel precipitin reactions, immunodiffusion (single or double), immunoelectrophoresis, radioimmunoassay (RIA), enzyme-linked immunosorbent assays (ELISAs), immunofluorescent assays, Western blotting, and the like.

In one preferred embodiment, the CRF, and/or CRF-BP, and/or CRF2 receptor polypeptide(s) are detected/quantified in an electrophoretic protein separation (e.g. a 1- or 2-dimensional electrophoresis). Means of detecting proteins using electrophoretic techniques are well known to those of skill in the art (see generally, R. Scopes (1982) Protein Purification, Springer-Verlag, N.Y.; Deutscher, (1990) Methods in Enzymology Vol. 182: Guide to Protein Purification, Academic Press, Inc., N.Y.).

In another preferred embodiment, Western blot (immunoblot) analysis is used to detect and quantify the presence of polypeptide(s) of this invention in the sample. This technique generally comprises separating sample proteins by gel electrophoresis on the basis of molecular weight, transferring the separated proteins to a suitable solid support, (such as a nitrocellulose filter, a nylon filter, or derivatized nylon filter), and incubating the sample with the antibodies that specifically bind the target polypeptide(s).

The antibodies specifically bind to the target polypeptide(s) and may be directly labeled or alternatively may be subsequently detected using secondary labeled antibodies (e.g., labeled sheep anti-mouse antibodies) that specifically bind to a domain of the primary antibody.

In preferred embodiments, the CRF, and/or CRF-BP, and/or CRF2 receptor polypeptide(s) are detected using an immunoassay. As used herein, an immunoassay is an assay that utilizes an antibody to specifically bind to the analyte (e.g., the target polypeptide(s)). The immunoassay is thus characterized by detection of specific binding of a polypeptide of this invention to an antibody as opposed to the use of other physical or chemical properties to isolate, target, and quantify the analyte.

Any of a number of well-recognized immunological binding assays (see, e.g., U.S. Pat. Nos. 4,366,241; 4,376,110; 4,517,288; and 4,837,168) are well suited to detection or quantification of the polypeptide(s) identified herein. For a review of the general immunoassays, see also Asai (1993) Methods in Cell Biology Volume 37: Antibodies in Cell Biology, Academic Press, Inc. New York; Stites & Terr (1991) Basic and Clinical Immunology 7th Edition.

Immunological binding assays (or immunoassays) typically utilize a “capture agent” to specifically bind to and often immobilize the analyte (e.g., CRF, and/or CRF-BP, and/or CRF2 receptor proteins). In preferred embodiments, the capture agent is an antibody.

Immunoassays also often utilize a labeling agent to specifically bind to and label the binding complex formed by the capture agent and the analyte. The labeling agent may itself be one of the moieties comprising the antibody/analyte complex. Thus, the labeling agent may be a labeled polypeptide or a labeled antibody that specifically recognizes the already bound target polypeptide. Alternatively, the labeling agent may be a third moiety, such as another antibody, that specifically binds to the capture agent/polypeptide complex.

Other proteins capable of specifically binding immunoglobulin constant regions, such as protein A or protein G may also be used as the labeling agent. These proteins are normal constituents of the cell walls of streptococcal bacteria. They exhibit a strong non-immunogenic reactivity with immunoglobulin constant regions from a variety of species (see, generally Kronval, et al. (1973) J. Immunol., 111: 1401-1406, and Akerstrom (1985) J. Immunol., 135: 2589-2542).

Preferred immunoassays for detecting the target polypeptide(s) are either competitive or noncompetitive. Noncompetitive immunoassays are assays in which the amount of captured analyte is directly measured. In one preferred “sandwich” assay, for example, the capture agents (antibodies) can be bound directly to a solid substrate where they are immobilized. These immobilized antibodies then capture the target polypeptide present in the test sample. The target polypeptide thus immobilized is then bound by a labeling agent, such as a second antibody bearing a label.

In competitive assays, the amount of analyte (e.g. CRF, and/or CRF-BP, and/or CRF2 receptor protein) present in the sample is measured indirectly by measuring the amount of an added (exogenous) analyte displaced (or competed away) from a capture agent (antibody) by the analyte present in the sample. In one competitive assay, a known amount of, in this case, labeled polypeptide is added to the sample and the sample is then contacted with a capture agent. The amount of labeled polypeptide bound to the antibody is inversely proportional to the concentration of target polypeptide present in the sample.

In one particularly preferred embodiment, the antibody is immobilized on a solid substrate. The amount of target polypeptide bound to the antibody may be determined either by measuring the amount of target polypeptide present in an polypeptide/antibody complex, or alternatively by measuring the amount of remaining uncomplexed polypeptide.

The immunoassay methods of the present invention include an enzyme immunoassay (EIA) which utilizes, depending on the particular protocol employed, unlabeled or labeled (e.g., enzyme-labeled) derivatives of polyclonal or monoclonal antibodies or antibody fragments or single-chain antibodies that bind CRF, and/or CRF-BP, and/or CRF2 receptor, either alone or in combination. In the case where the antibody that binds the CRF, and/or CRF-BP, and/or CRF2 receptor polypeptide(s) is not labeled, a different detectable marker, for example, an enzyme-labeled antibody capable of binding to the monoclonal antibody which binds the CRF, and/or CRF-BP, and/or CRF2 receptor polypeptide, can be employed. Any of the known modifications of EIA, for example, enzyme-linked immunoabsorbent assay (ELISA), may also be employed. As indicated above, also contemplated by the present invention are immunoblotting immunoassay techniques such as Western blotting employing an enzymatic detection system.

The immunoassay methods of the present invention can also include other known immunoassay methods, for example, fluorescent immunoassays using antibody conjugates or antigen conjugates of fluorescent substances such as fluorescein or rhodamine, latex agglutination with antibody-coated or antigen-coated latex particles, haemagglutination with antibody-coated or antigen-coated red blood corpuscles, and immunoassays employing an avidin-biotin or strepavidin-biotin detection systems, and the like.

The particular parameters employed in the immunoassays of the present invention can vary widely depending on various factors such as the concentration of antigen in the sample, the nature of the sample, the type of immunoassay employed and the like. Optimal conditions can be readily established by those of ordinary skill in the art. In certain embodiments, the amount of antibody that binds the CRF, and/or CRF-BP, and/or CRF2 receptor polypeptide is typically selected to give 50% binding of detectable marker in the absence of sample. If purified antibody is used as the antibody source, the amount of antibody used per assay will generally range from about 1 ng to about 100 ng. Typical assay conditions include a temperature range of about 4° C. to about 45° C., preferably about 25° C. to about 37° C., and most preferably about 25° C., a pH value range of about 5 to 9, preferably about 7, and an ionic strength varying from that of distilled water to that of about 0.2M sodium chloride, preferably about that of 0.15M sodium chloride. Times will vary widely depending upon the nature of the assay, and generally range from about 0.1 minute to about 24 hours. A wide variety of buffers, for example PBS, may be employed, and other reagents such as salt to enhance ionic strength, proteins such as serum albumins, stabilizers, biocides and non-ionic detergents can also be included.

The assays of this invention are scored (as positive or negative or quantity of target polypeptide) according to standard methods well known to those of skill in the art. The particular method of scoring will depend on the assay format and choice of label. For example, a Western Blot assay can be scored by visualizing the colored product produced by the enzymatic label. A clearly visible colored band or spot at the correct molecular weight is scored as a positive result, while the absence of a clearly visible spot or band is scored as a negative. The intensity of the band or spot can provide a quantitative measure of target polypeptide concentration.

Antibodies for use in the various immunoassays described herein are commercially available or can be produced using standard methods well known to those of skill in the art.

It will also be recognized that antibodies can be prepared by any of a number of commercial services (e.g., Berkeley Antibody Laboratories, Bethyl Laboratories, Anawa, Eurogenetec, etc.).

3) Assay Optimization.

The assays of this invention have immediate utility in screening for agents that modulate the expression or activity of CRF, and/or CRF-BP, and/or CRF2 receptor by a cell, tissue or organism. The assays of this invention can be optimized for use in particular contexts, depending, for example, on the source and/or nature of the biological sample and/or the particular test agents, and/or the analytic facilities available. Thus, for example, optimization can involve determining optimal conditions for binding assays, optimum sample processing conditions (e.g. preferred PCR conditions), hybridization conditions that maximize signal to noise, protocols that improve throughput, etc. In addition, assay formats can be selected and/or optimized according to the availability of equipment and/or reagents. Thus, for example, where commercial antibodies or ELISA kits are available, it may be desired to assay protein concentration. Conversely, where it is desired to screen for modulators that alter transcription the CRF, and/or CRF-BP, and/or CRF2 receptor gene(s), nucleic acid based assays are preferred.

Routine selection and optimization of assay formats are well known to those of ordinary skill in the art.

III. Pre-Screening for Agents that Bind a CRF and/or CRF-BP, and/or CRF2R and/or Complexes Thereof.

In certain embodiments it is desired to pre-screen test agents for the ability to interact with (e.g. specifically bind to) CRF and/or CRF-BP, and/or CRF2R and/or complexes thereof or to a nucleic acid encoding CRF, CRF-BP, and/or CRF2R. Specifically binding test agents are more likely to interact with one or more of these components and thereby modulate CRF potentiation of NMDA receptors. Thus, in some preferred embodiments, the test agent(s) are pre-screened for binding to CRF and/or CRF-BP, and/or CRF2R and/or complexes thereof or to a nucleic acid encoding CRF, CRF-BP, and/or CRF2R before performing the more complex assays described above.

In one embodiment, such pre-screening is accomplished with simple binding assays. Means of assaying for specific binding or the binding affinity of a particular ligand for a nucleic acid or for a protein are well known to those of skill in the art. In preferred binding assays, the CRF and/or CRF-BP, and/or CRF2R and/or complexes thereof or the nucleic acid encoding CRF and/or CRF-BP and/or CRF2R is immobilized and exposed to a test agent (which can be labeled), or alternatively, the test agent(s) are immobilized and exposed to CRF and/or CRF-BP and/or CRF2R and/or complexes thereof or to a nucleic acid encoding CRF and/or CRF-BP and/or CRF2R (which can be labeled). The immobilized moiety is then washed to remove any unbound material and the bound test agent or bound protein or nucleic acid is detected (e.g. by detection of a label attached to the bound molecule). The amount of immobilized label is proportional to the degree of binding between the test agent and the CRF and/or CRF-BP and/or CRF2R and/or complexes thereof or to a nucleic acid encoding CRF and/or CRF-BP and/or CRF2R.

IV. Scoring the Assays.

As indicated above, methods of screening for modulators of orexin, orexin receptor, CRF and/or CRF-BP and/or CRF receptor expression, interaction, or activity typically involve contacting a cell, tissue, organism, animal with one or more test agents and evaluating changes in orexin, orexin receptor, CRF and/or CRF-BP and/or CRF receptor nucleic acid transcription and/or translation or orexin, orexin receptor, CRF and/or CRF-BP and/or CRF receptor protein activity or interaction. To screen for potential modulators, the assays described above are typically performed using biological samples from cells and/or tissues and/or organs and/or organisms exposed to one or more test agents. The orexin, orexin receptor, CRF and/or CRF-BP and/or CRF receptor expression, activity, or interaction is determined and, in a preferred embodiment, compared to the corresponding level(s) observed in “control” assays (e.g., the same assays lacking the test agent). A difference in the “test” level(s) as compared to the control level(s) indicates that the test agent is a “modulator” of orexin, orexin receptor, CRF and/or CRF-BP and/or CRF receptor expression, activity, or interaction and consequently a modulator of NMDA receptor mediated currents.

In a preferred embodiment, the assays of this invention are deemed to show a positive result, e.g. elevated expression and/or activity and/or interaction of orexin, orexin receptor, CRF and/or CRF-BP and/or CRF receptor, when the measured protein or nucleic acid level, protein activity, or protein interaction is greater than the level measured or known for a control sample (e.g. either a level known or measured for a normal healthy cell, tissue or organism mammal of the same species not exposed to the or putative modulator (test agent) or a “baseline/reference” level determined in a different tissue and/or at a different time for the same individual). In a particularly preferred embodiment, the assay is deemed to show a positive result when the difference between sample and “control” is statistically significant (e.g. at the 85% or greater, preferably at the 90% or greater, more preferably at the 95% or greater and most preferably at the 98% or greater confidence level).

V. High Throughput Screening.

The assays of this invention are also amenable to “high-throughput” modalities. Conventionally, new chemical entities with useful properties (e.g., modulation of orexin, orexin receptor, CRF and/or CRF-BP and/or CRF receptor expression, activity, or interaction) are generated by identifying a chemical compound (called a “lead compound”) with some desirable property or activity, creating variants of the lead compound, and evaluating the property and activity of those variant compounds. However, the current trend is to shorten the time scale for all aspects of drug discovery. Because of the ability to test large numbers quickly and efficiently, high throughput screening (HTS) methods are replacing conventional lead compound identification methods.

In one preferred embodiment, high throughput screening methods involve providing a library containing a large number of compounds (candidate compounds) potentially having the desired activity. Such “combinatorial chemical libraries” are then screened in one or more assays, as described herein, to identify those library members (particular chemical species or subclasses) that display a desired characteristic activity. The compounds thus identified can serve as conventional “lead compounds” or can themselves be used as potential or actual therapeutics.

A) Combinatorial Chemical Libraries

The use of combinatorial chemical libraries assists greatly in the generation of new chemical compound leads. A combinatorial chemical library is a collection of diverse chemical compounds generated by either chemical synthesis or biological synthesis by combining a number of chemical “building blocks” such as reagents. For example, a linear combinatorial chemical library such as a polypeptide library is formed by combining a set of chemical building blocks called amino acids in every possible way for a given compound length (i.e., the number of amino acids in a polypeptide compound). Millions of chemical compounds can be synthesized through such combinatorial mixing of chemical building blocks. For example, one commentator has observed that the systematic, combinatorial mixing of 100 interchangeable chemical building blocks results in the theoretical synthesis of 100 million tetrameric compounds or 10 billion pentameric compounds (Gallop et al. (1994) 37(9): 1233-1250).

Preparation and screening of combinatorial chemical libraries is well known to those of skill in the art. Such combinatorial chemical libraries include, but are not limited to, peptide libraries (see, e.g., U.S. Pat. No. 5,010,175, Furka (1991) Int. J. Pept. Prot. Res., 37: 487-493, Houghton et al. (1991) Nature, 354: 84-88). Peptide synthesis is by no means the only approach envisioned and intended for use with the present invention. Other chemistries for generating chemical diversity libraries can also be used. Such chemistries include, but are not limited to: peptoids (PCT Publication No WO 91/19735, 26 Dec. 1991), encoded peptides (PCT Publication WO 93/20242, 14 Oct. 1993), random bio-oligomers (PCT Publication WO 92/00091, 9 Jan. 1992), benzodiazepines (U.S. Pat. No. 5,288,514), diversomers such as hydantoins, benzodiazepines and dipeptides (Hobbs et al., (1993) Proc. Nat. Acad. Sci. USA 90: 6909-6913), vinylogous polypeptides (Hagihara et al. (1992) J. Amer. Chem. Soc. 114: 6568), nonpeptidal peptidomimetics with a Beta-D-Glucose scaffolding (Hirschmann et al., (1992) J. Amer. Chem. Soc. 114: 9217-9218), analogous organic syntheses of small compound libraries (Chen et al. (1994) J. Amer. Chem. Soc. 116: 2661), oligocarbamates (Cho, et al., (1993) Science 261:1303), and/or peptidyl phosphonates (Campbell et al., (1994) J. Org. Chem. 59: 658) (see, generally, Gordon et al., (1994) J. Med. Chem. 37:1385, nucleic acid libraries (see, e.g., Strategene, Corp.), peptide nucleic acid libraries (see, e.g., U.S. Pat. No. 5,539,083) antibody libraries (see, e.g., Vaughn et al. (1996) Nature Biotechnology, 14(3): 309-314) and PCT/US96/10287), carbohydrate libraries (see, e.g., Liang et al. (1996) Science, 274: 1520-1522, and U.S. Pat. No. 5,593,853), and small organic molecule libraries (see, e.g., benzodiazepines, Baum (1993) C&EN, Jan 18, page 33, isoprenoids U.S. Pat. No. 5,569,588, thiazolidinones and metathiazanones U.S. Pat. No. 5,549,974, pyrrolidines U.S. Pat. Nos. 5,525,735 and 5,519,134, morpholino compounds U.S. Pat. No. 5,506,337, benzodiazepines 5,288,514, and the like).

Devices for the preparation of combinatorial libraries are commercially available (see, e.g., 357 MPS, 390 MPS, Advanced Chem Tech, Louisville Ky., Symphony, Rainin, Woburn, Mass., 433A Applied Biosystems, Foster City, Calif., 9050 Plus, Millipore, Bedford, Mass.).

A number of well known robotic systems have also been developed for solution phase chemistries. These systems include automated workstations like the automated synthesis apparatus developed by Takeda Chemical Industries, LTD. (Osaka, Japan) and many robotic systems utilizing robotic arms (Zymate II, Zymark Corporation, Hopkinton, Mass.; Orca, Hewlett-Packard, Palo Alto, Calif.) which mimic the manual synthetic operations performed by a chemist. Any of the above devices are suitable for use with the present invention. The nature and implementation of modifications to these devices (if any) so that they can operate as discussed herein will be apparent to persons skilled in the relevant art. In addition, numerous combinatorial libraries are themselves commercially available (see, e.g., ComGenex, Princeton, N.J., Asinex, Moscow, Ru, Tripos, Inc., St. Louis, Mo., ChemStar, Ltd, Moscow, RU, 3D Pharmaceuticals, Exton, Pa., Martek Biosciences, Columbia, Md., etc.).

B) High Throughput Assays of Chemical Libraries.

Any of the assays for agents that modulate expression, activity or interaction of orexin, orexin receptor, CRF and/or CRF-BP and/or CRF receptor are amenable to high throughput screening. As described above, having determined that orexin, orexin receptor, CRF and/or CRF-BP and/or CRF receptor are associated with potentiation of NMDA receptors, it is believe that modulators can have significant therapeutic value. Certain preferred assays detect increases of transcription (i.e., increases of mRNA production) by the test compound(s), increases of protein expression by the test compound(s), or binding to the gene (e.g., gDNA, or cDNA) or gene product (e.g., mRNA or expressed protein) by the test compound(s). Alternatively, the assay can detect inhibition of the characteristic activity of the orexin, orexin receptor, CRF and/or CRF-BP and/or CRF receptor.

High throughput assays for the presence, absence, or quantification of particular nucleic acids or protein products are well known to those of skill in the art. Binding assays are similarly well known. Thus, for example, U.S. Pat. No. 5,559,410 discloses high throughput screening methods for proteins, U.S. Pat. No. 5,585,639 discloses high throughput screening methods for nucleic acid binding (i.e., in arrays), while U.S. Pat. Nos. 5,576,220 and 5,541,061 disclose high throughput methods of screening for ligand/antibody binding.

In addition, high throughput screening systems are commercially available (see, e.g., Zymark Corp., Hopkinton, Mass.; Air Technical Industries, Mentor, Ohio; Beckman Instruments, Inc. Fullerton, Calif.; Precision Systems, Inc., Natick, Mass., etc.). These systems typically automate entire procedures including all sample and reagent pipetting, liquid dispensing, timed incubations, and final readings of the microplate in detector(s) appropriate for the assay. These configurable systems provide high throughput and rapid start up as well as a high degree of flexibility and customization. The manufacturers of such systems provide detailed protocols of the various high throughput assays. Thus, for example, Zymark Corp. provides technical bulletins describing screening systems for detecting the modulation of gene transcription, ligand binding, and the like.

VI. Kits.

In still another embodiment, this invention provides kits for practice of the assays or use of the compositions described herein. In one preferred embodiment, the kits comprise one or more containers containing antibodies and/or nucleic acid probes and/or substrates suitable for detection of orexin, orexin receptor (e.g., OXR1), CRF and/or CRF-BP and/or CRF receptor (e.g., CRF2) expression and/or activity and/or interaction levels. The kits can optionally include any reagents and/or apparatus to facilitate practice of the assays described herein. Such reagents include, but are not limited to buffers, labels, labeled antibodies, labeled nucleic acids, filter sets for visualization of fluorescent labels, blotting membranes, and the like.

In another embodiment, the kits can comprise a container containing an orexin, orexin receptor, CRF and/or CRF-BP and/or CRF receptor protein(s), and/or a vector encoding an orexin, orexin receptor, CRF and/or CRF-BP and/or CRF receptor, and/or a cell comprising such a vector.

In addition, the kits can optionally include instructional materials containing directions (i.e., protocols) for the practice of the assay methods of this invention or the administration of the compositions described here along with counterindications. While the instructional materials typically comprise written or printed materials they are not limited to such. Any medium capable of storing such instructions and communicating them to an end user is contemplated by this invention. Such media include, but are not limited to electronic storage media (e.g., magnetic discs, tapes, cartridges, chips), optical media (e.g., CD ROM), and the like. Such media may include addresses to internet sites that provide such instructional materials.

VII. Modulator Databases.

In certain embodiments, the agents that score positively in the assays described herein (e.g. show an ability to inhibit and/or to increase the expression and/or activity, and/or interaction of orexin, orexin receptor, CRF and/or CRF-BP and/or CRF receptor) can be entered into a database of putative modulators of NMDA currents.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### EXAMPLES

### Example 1

### Orexin A in the VTA is Critical for the Induction of Synaptic Plasticity and Behavioral Sensitization to Cocaine

Summary

Dopamine neurons in the ventral tegmental area (VTA) represent a critical site of synaptic plasticity induced by addictive drugs. Orexin/hypocretin-containing neurons in the lateral hypothalamus project to the VTA, and behavioral studies have suggested that orexin neurons play a critical role in motivation, feeding and adaptive behaviors. However, the role of orexin signaling in neural plasticity is poorly understood. The present study shows that in vitro application of orexin A induces potentiation of N-methyl-D-aspartate receptor-(NMDAR) mediated neurotransmission via a PLC/PKC-dependent insertion of NMDARs in VTA dopamine neuron synapses. Furthermore, in vivo administration of an orexin 1 receptor antagonist blocks locomotor sensitization to cocaine and occludes cocaine-induced potentiation of excitatory currents in VTA dopamine neurons. These results provide in vitro and in vivo evidence for a critical role for orexin signaling in the VTA in neural plasticity relevant to addiction.

Abbreviations:

AMPAR α-aspartate-3-hydroxy-5-methyl-4 isoxazole propionate receptor

D-AP5 D (−)-2-Amino-5-phosphonopentanoic acid

EPSC excitatory post-synaptic current

FITC fluorescein isothiocyanate

LH lateral hypothalamus

LTP long-term potentiation

NMDAR N-methyl-D-aspartate receptor

OXR1 orexin receptor 1

OXR2 orexin receptor 2

PLC phospholipase C

PKC protein kinase C

TH tyrosine hydroxylase

TR texas red

VTA ventral tegmental area

Introduction

Drugs of abuse can alter synaptic plasticity in the mesolimbic dopamine system, a region that is implicated in a variety of addictive behaviors. In particular, long lasting changes at excitatory synapses in the nucleus accumbens and the VTA result from in vivo administration of drugs of abuse (Fitzgerald et al., 1996; Thomas et al., 2001; Ungless et al., 2001; Borgland et al., 2004). The lateral hypothalamus (LH) sends a substantial projection to the VTA (Fadel and Deutch, 2002; Baldo et al., 2003) and is a critical element in motivation and reward circuits activated by drugs of abuse, including cocaine (Wise, 1996).

Orexins (hypocretins) are neuropeptides synthesized in neurons of the LH that can elicit arousal, feeding and appetitive behaviors (DeLecea et al., 1998; Sakurai et al., 1998). There are two known orexins, A and B, whose actions are mediated by two G protein-coupled receptors termed orexin receptor type 1 (OXR1) and type 2 (OXR2); OXR1 shows higher affinity for orexin A, while OXR2 shows equal affinity for the two ligands (Sakurai et al., 1998).

Dopamine neurons in the VTA are critical for a similar repertoire of motivated behaviors. In addition, excitatory synaptic transmission in VTA dopamine neurons is an important locus of neural plasticity induced by psychostimulant administration. Several lines of evidence suggest that orexin modulates dopaminergic neurotransmission. First, terminals of LH orexin neurons are apposed to dendrites and somata of dopaminergic VTA neurons (Fadel and Deutch, 2002). Second, the dopamine receptor antagonist haloperidol blocks hyper-locomotion and stereotypy induced by intracerebroventricular orexin (Nakamura et al., 2000). Finally, orexin increases the firing rate of VTA neurons (Korotkova et al., 2003). Taken together, these results suggest that endogenous orexin signaling in dopamine neurons is an important substrate for the expression of motivated behaviors. However, the contribution of orexin signaling to VTA neural plasticity had not been investigated.

Glutamatergic transmission in the VTA plays a key role in neural plasticity relevant to addiction (Carlezon and Nestler, 2002; Kauer, 2004). In particular, the induction of behavioral sensitization, a behavioral model used to assess addictive substances, is dependent upon activation of N-methyl-D-aspartate receptors (NMDARs) in the VTA (Vanderschuren and Kalivas, 2000). NMDARs mediate long-term plasticity at a variety of excitatory synapses, and their activation promotes burst firing in VTA neurons and optimizes dopamine release (Komendantov et al., 2004). To elucidate potential contributions of orexin signaling to neural plasticity in the VTA, the effects of orexin A signaling on excitatory synapses of VTA dopamine neurons were examined.

Results

Orexin A potentiates NMDAR EPSCs in Dopamine Neurons.

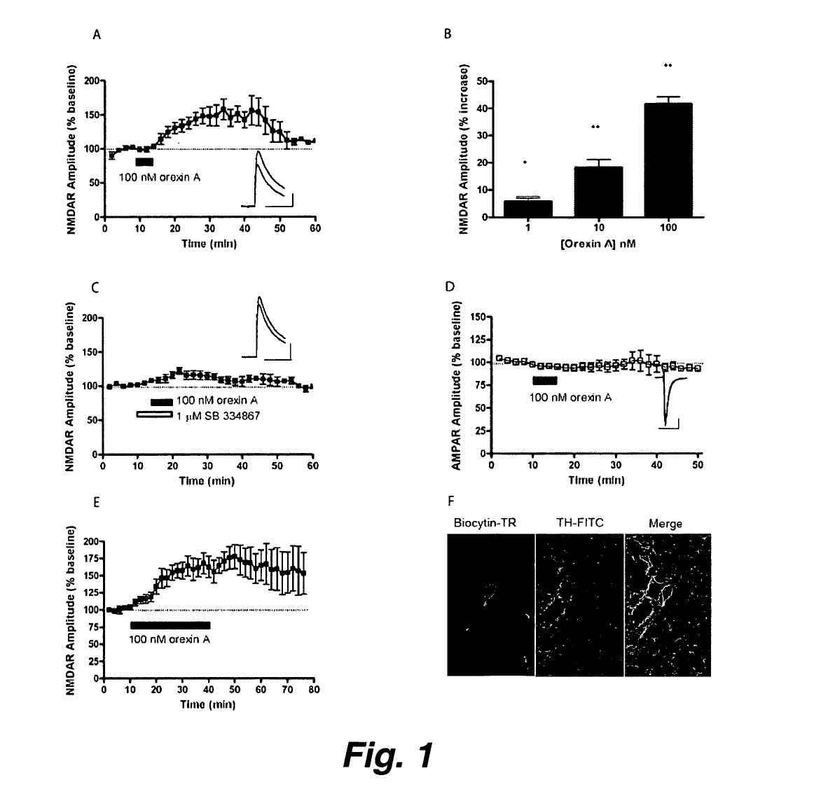

Excitatory postsynaptic currents (EPSCs) were recorded from VTA neurons in rat midbrain slices. EPSCs were evoked while holding neurons in voltage clamp at +40 mV. Measurements were taken 20 ms after the stimulus artifact, a time point at which the glutamatergic EPSC is mediated purely by NMDARs. Bath application of orexin A for 5 min resulted in a 49±9% potentiation of NMDAR-mediated EPSCs (FIG. 1A, n=12, 10/12 neurons had potentiation greater than 10%). This potentiation was concentration-dependent (FIG. 1B, 1 nM: 5±1%, n=6; 10 nM: 18±3%, n=6; 100 nM: 42±5%, n=6) and was significantly inhibited by the selective OXR1 receptor antagonist SB 334867 (FIG. 1C, 1 μM, n=8, p<0.01) although not completely blocked, suggesting the presence of OXR2 as well. AMPAR-mediated EPSCs recorded at −70 mV were not changed by orexin A (FIG. 1D, n=8). Application of orexin A (100 nM) for 30 min resulted in a 74±16% potentiation of NMDAR EPSCs that lasted longer than 40 min after washout (FIG. 1E, n=6). Orexin A-induced potentiation of NMDAR EPSCs, but not AMPAR EPSCs, suggests that orexin-mediated potentiation occurs through a post-synaptic mechanism.

The VTA is composed of a heterogeneous collection of cell types, distinguished in part by neurotransmitter content. In addition to principal neurons, which are mostly dopaminergic, there are secondary and tertiary neurons that are mostly GABAergic (Cameron et al., 1997; Margolis et al., 2003). To determine whether the neurons showing orexin A-induced potentiation were indeed dopaminergic, we filled neurons with biocytin while recording EPSCs and subsequently processed slices for tyrosine hydroxylase (TH). In TH-containing neurons, orexin A potentiated NMDAR responses in 7/8 neurons (FIG. 1F). Of the 2 neurons that did not express TH, neither responded to orexin A. Thus, orexin A-induced potentiation of NMDAR-mediated EPSCs is found primarily in VTA dopamine-containing neurons.

Orexin A Potentiates NMDAR EPSCs Via a PKC/PLC-Dependent Mechanism.

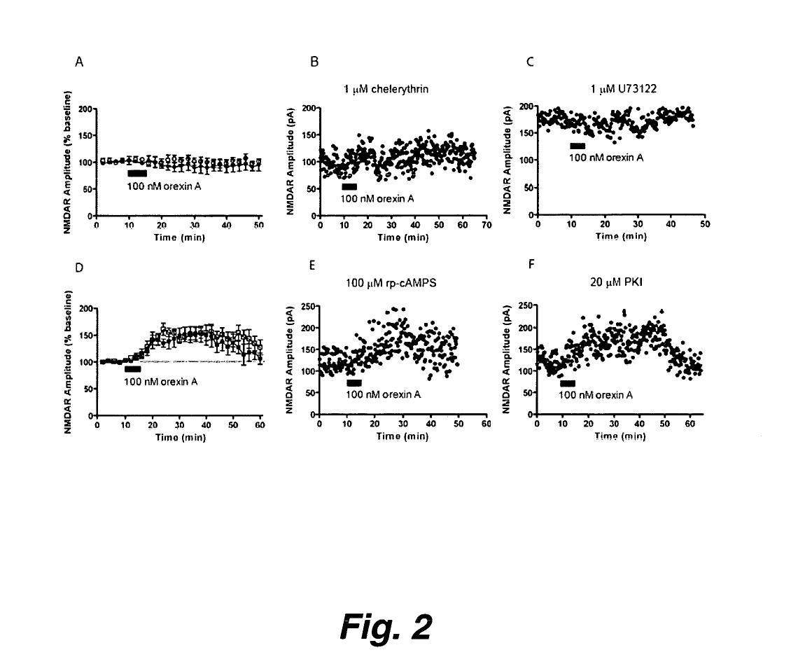

The aim of the next set of experiments was to identify the intracellular pathway through which OXR1 activation leads to NMDAR potentiation. The patch pipette was loaded with the PLC inhibitor, U-73122 (1 μM, FIG. 2A, C, n=6) or the PKC inhibitor, chelerythrin (1 μM, FIG. 1A, B, n=6). In both cases, the potentiation of NMDAR-mediated EPSCs by 100 nM orexin A was completely blocked. Next, the protein kinase A inhibitor, PKI (20 μM, FIG. 2A, F, n=8) or Rp-cAMPs, a blocker of cAMP (100 μM, FIG. 2A, E, n=5) was added to the patch pipette. In these experiments, robust potentiation of NMDAR-mediated EPSCs occurred after orexin A application. Taken together, these data indicate that orexin A potentiates NMDARs in VTA dopamine neurons through a PLC/PKC-dependent pathway.

Orexin A Modulates NMDAR Subunit Composition.

NMDARs are composed of an obligatory NR1 subunit and at least one NR2A, B, C or D subunit (Cull-Candy et al., 2001). Changes in NMDAR subunit composition confer distinct gating and pharmacological properties on heteromeric NMDARs (Cull-Candy et al., 2001) and a switch in subunit composition may alter synaptic function (Liu et al., 2004; Erreger et al., 2005). Therefore, it was of interest to determine which type of NR2 subunit mediates orexin A-induced potentiation of NMDA responses.

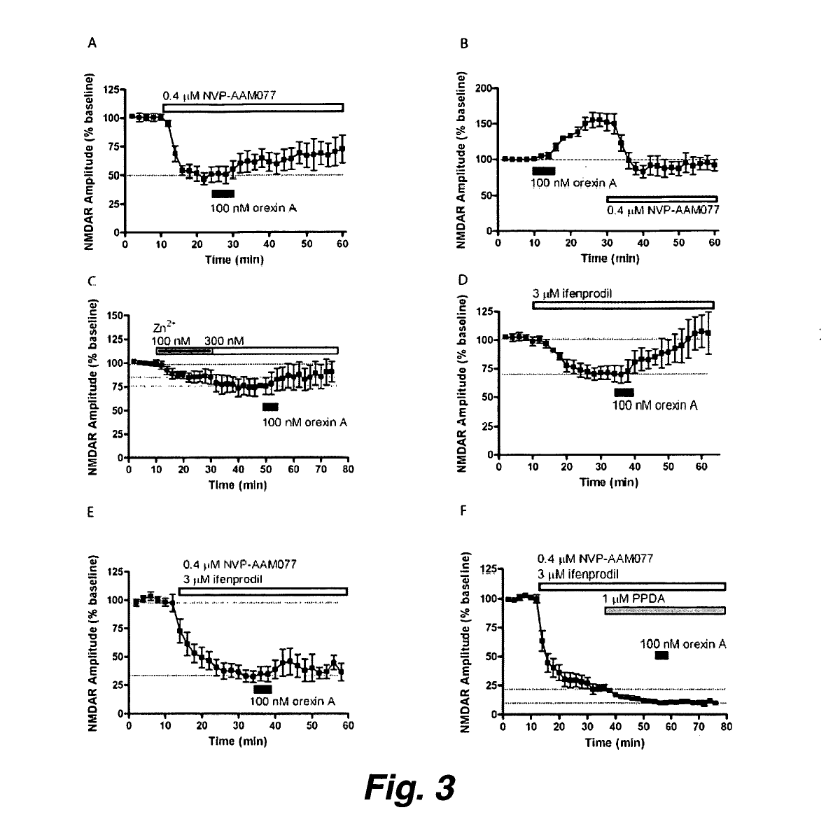

Bath application of the NR2A/C antagonist, NVP-AAM077 (0.4 μM; Liu et al., 2004; PEAQX, Feng et al., 2004) inhibited NMDAR EPSCs by 54±7% and did not cause any further decrease of the current over the duration of application (40 min, n=6). The ability of 100 nM orexin A to potentiate NMDAR-mediated EPSCs was significantly reduced in the presence of NVP-AAM077 (FIG. 3A, n=7, maximum potentiation=20±6%, p<0.05). To determine if the NR2A/C antagonist could block expression of orexin A-mediated potentiation of NMDAR EPSCs, NVP-AAM077 (0.4 μM) was applied during the peak increase of NMDAR EPSCs induced by 100 nM orexin A application. Accordingly, NVP-AAM077 blocked orexin-mediated potentiation of NMDAR EPSCs (FIG. 3B, n=6, remaining current=87±11%). Further, the effects of orexin A were tested in the presence of Zn²⁺, a high-affinity selective inhibitor of NR1-NR2A receptors (Paoletti et al., 1997). Zn²⁺ inhibited NMDAR EPSCs by 13±8% (100 nM, FIG. 3C, n=6) and by 29±9% (300 nM, FIG. 3C, n=5, p<0.05). Potentiation of NMDARs was significantly reduced in the presence of Zn²⁺ (FIG. 3C, n=5, maximum potentiation=14±4%, p<0.05). To determine the potential involvement of NR2B-containing subunits in orexin A-mediated potentiation of NMDAR EPSCs, orexin A responses were measured in the presence of the bath-applied NR2B antagonist, ifenprodil (3 μM). The remaining current after ifenprodil application was 71±6%. In the absence of NR2B-containing NMDAR, orexin A (100 nM) potentiated NMDAR-mediated EPSCs by 36% to a maximum amplitude of 107±16% (FIG. 3D, n=6) of baseline EPSCs. When both NR2A/C and NR2B inhibitors were co-applied, orexin A (100 nM) potentiation of NMDAR EPSCs was largely inhibited (FIG. 3E, n=6, orexin potentiation: 32±5% to 45±12%, p>0.05). To determine if the residual potentiation observed in the presence of NVP-AAM077 or Zn²⁺ could be blocked, the effects of orexin A were recorded after co-application of the NR2A/C and NR2B inhibitors in addition to the NR2C/D-preferring antagonist, PPDA (1 μM; Feng, et al., 2004). PPDA further inhibited NMDAR EPSCs by an additional 10% (remaining current 14±2.2%, n=6, FIG. 3F) and orexin A (100 nM) did not alter the remaining current in the presence of these blockers.

Orexin A Translocates NMDARs to the Synapse.

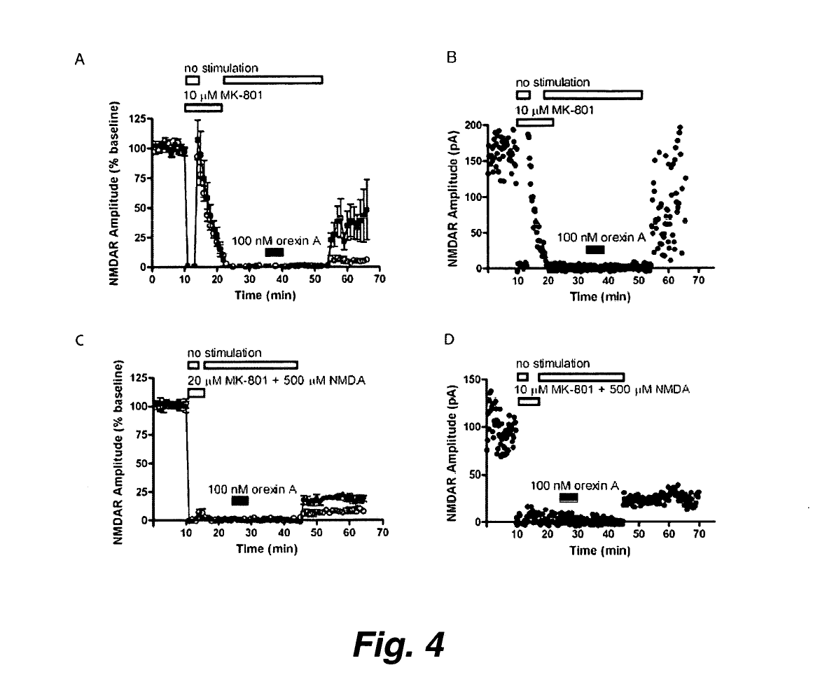

Activation of PKC is implicated in NMDAR-dependent long-term potentiation (Malinow et al., 1989) and it modulates NMDAR trafficking to the membrane (Lan et al., 2001; Scott et al., 2001; Fong et al., 2002). NMDARs can translocate from intracellular or extrasynaptic pools to synaptic sites (Lan et al., 2001; Tovar and Westbrook, 2002). Therefore, orexin A-mediated potentiation of NMDAR EPSCs might be due to movement of NMDARs from intracellular or extrasynaptic sites to the synapse. Because MK-801 is an activity-dependent and irreversible NMDAR antagonist (Rosemund et al., 1993), this agent was used to block only synaptic NMDARs that opened in response to synaptically-released glutamate. MK-801 (10 μM; Tovar and Westbrook, 2002) was applied to VTA slices in the absence of stimulation to equilibrate bath concentration. MK-801 progressively blocked NMDAR-EPSCs during the 0.1 Hz stimulation. Approximately 5% of the current remained after MK-801 application. The unbound MK-801 was then washed out in the absence of stimulation for 40 min in control neurons (n=5). In a separate group of slices, orexin A (100 nM, n=6) was applied for 5 min during the washout period. Next, synaptic activity was stimulated, with the result that the maximal NMDAR amplitude in orexin A-treated neurons was significantly greater than in controls (FIG. 4A,B, maximum current orexin: 40±17%, control: 5±1%, p<0.05). In another set of experiments, NMDA (500 μM) was first co-applied with MK-801 (20 μM) to block all synaptic and extrasynaptic NMDARs. Then, orexin A was applied for 5 min during the washout of MK-801 and NMDA in the absence of stimulation (FIG. 4C,D). After 15 min washout of orexin, synaptic activity was stimulated, with the result that the maximal NMDAR amplitude in orexin A-treated neurons was again significantly greater than in controls (FIG. 4C,D, maximum current orexin: 20±2%, n=6; control: 7±2%, n=6, p<0.05). These data indicate that orexin A potentiates NMDAR EPSCs, at least partially, by promoting movement of intracellular NMDARs to the synapse.

Orexin A Causes a Late Phase AMPAR-Mediated Plasticity and Facilitates Cocaine-Induced Potentiation of Excitatory Inputs to VTA Neurons.

Psychostimulants produce a form of experience-dependent plasticity known as behavioral sensitization, whereby animals exhibit a long-lasting increase in the locomotor-activating effects of the drug. This drug-induced locomotor effect is accompanied by increased motivation for drug intake and enhanced drug reward (Shippenberg and Heidbreder, 1995; Nestler, 2001; Kim et al., 2004). Behavioral sensitization is mediated by increased synaptic output of VTA dopamine neurons and alterations of dopamine and glutamate effects in the nucleus accumbens and prefrontal cortex (Vanderschuren and Kalivas, 2000). Potentiation of NMDARs is critical for the development of cocaine-mediated behavioral sensitization, and long-term plasticity at excitatory synapses can be observed with cocaine treatment (Vanderschuren and Kalivas, 2000). This long-term plasticity is evident as a persistent increase in the ratio of AMPA to NMDA-mediated synaptic currents of VTA neurons (Ungless et al., 2001; Saal et al., 2003; Borgland et al., 2004).

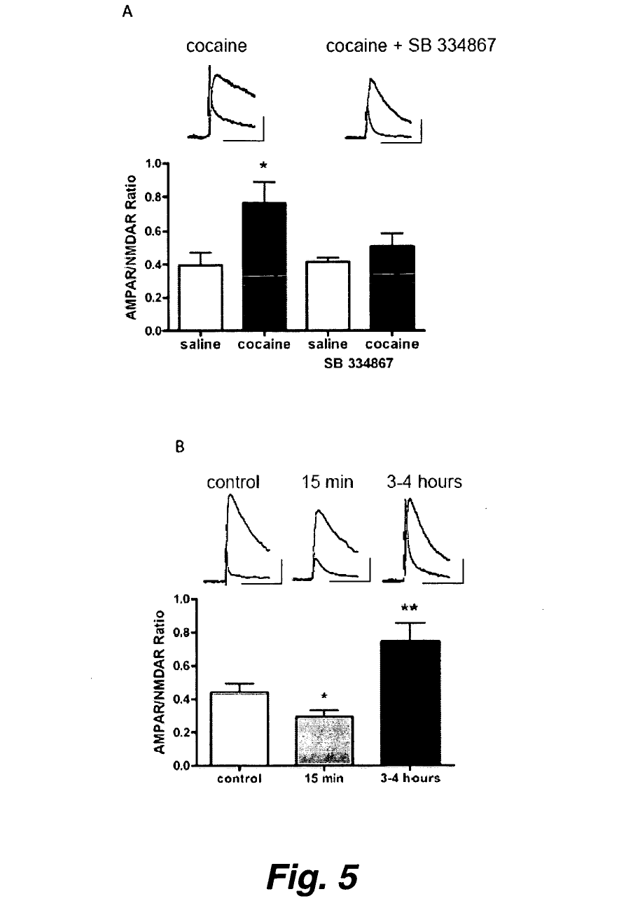

The next study investigated whether orexin signaling in the VTA is required for the long-term plasticity associated with the development of behavioral sensitization. Long-term synaptic plasticity at excitatory synapses on VTA neurons was examined 24 hr after a 5-day treatment of cocaine (15 mg/kg) or saline (0.9%), a time course shown to induce long-lasting locomotor sensitization to cocaine (Thomas et al., 2001), with or without the OXR1 antagonist, SB 334867 (10 mg/kg, FIG. 5). To assay for synaptic plasticity, the relative contributions of AMPARs and NMDARs to EPSCs recorded in VTA dopamine cells were compared. Consistent with the potentiation observed following acute and 7-day cocaine treatments (Borgland et al., 2004), 5-day treatment with cocaine potentiated the AMPAR/NMDAR ratio compared to saline controls (FIG. 5A, saline: 0.4±0.07, n=4; cocaine: 0.8±0.1, n=4, p<0.05). This potentiation of synaptic plasticity was blocked by 5-day treatment with the OXR1 antagonist, SB 334867, administered 15 min prior to cocaine or saline (FIG. 5A, saline+SB334867: 0.4±0.02, n=5 vs. cocaine+SB334867: 0.5±0.08, n=11, p>0.05). Since OXR1 antagonists block cocaine-mediated potentiation of the AMPAR/NMDAR ratio, orexin A may either facilitate cocaine-mediated synaptic plasticity or enhance synaptic transmission itself. A 5-min orexin A application was performed to determine whether this caused changes in AMPAR/NMDAR ratio either 15 min or 3-4 hours after application (FIG. 5B). Orexin A (100 nM) decreased AMPAR/NMDAR ratio 15 min after treatment (control: 0.44±0.06, n=8 vs. 15 min: 0.29±0.04, n=8, p<0.05), and significantly increased it after 3-4 hours (0.75±0.1, n=8, p<0.01, FIG. 5B).

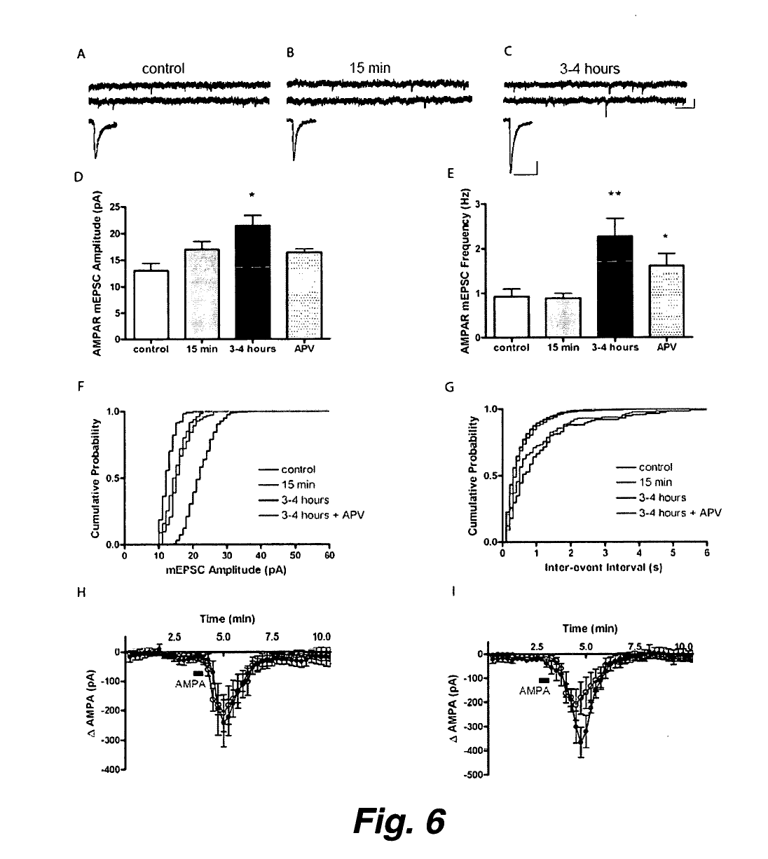

Next, to investigate whether AMPAR function and/or number was modified with orexin A treatment, AMPAR-mediated miniature EPSCs (mEPSCs) were examined (a standard method for determining the locus of synaptic change). Compared to untreated neurons, orexin A (100 nM) caused a significant increase in both amplitude (FIG. 6C,D,F) and frequency (FIG. 6C,E,G) 3-4 hours after application (24±1.6 pA; 2.4±0.5 Hz; n=7 vs control: 13±1.3 pA; 0.93±0.16 Hz; n=7; FIG. 6A), but not at 15 min (15±1.7 pA; 0.9±0.1 Hz; n=6, p<0.05, FIG. 6B). Interestingly, the increase in AMPAR mEPSC amplitude observed 3-4 hours after orexin A application was significantly reduced when the NMDAR antagonist APV (50 μM) was applied 3 min prior and during orexin application (16±0.6 pA, FIG. 6D, n=7, p>0.05). However, frequency remained elevated over controls 3-4 hours after orexin A application (1.6±0.3 Hz, FIG. 6E, n=7, p<0.05), suggesting that orexin application may also produce a distinct presynaptic effect.

When AMPA (2 μM for 30 sec) in the presence of cyclothiazide (100 μM; to reduce AMPAR desensitization) was superfused onto neurons 15 min after orexin A (100 nM) treatment, there was no significant increase in AMPAR-induced current (−243±79 pA, n=5) compared with untreated neurons (−210±64, n=9, FIG. 6H, p>0.05). AMPAR-induced currents significantly increased 3-4 hours after orexin A (100 nM) treatment (−367±63 pA, n=6, p<0.05, FIG. 6I, t-test of peak control vs. peak orexin).

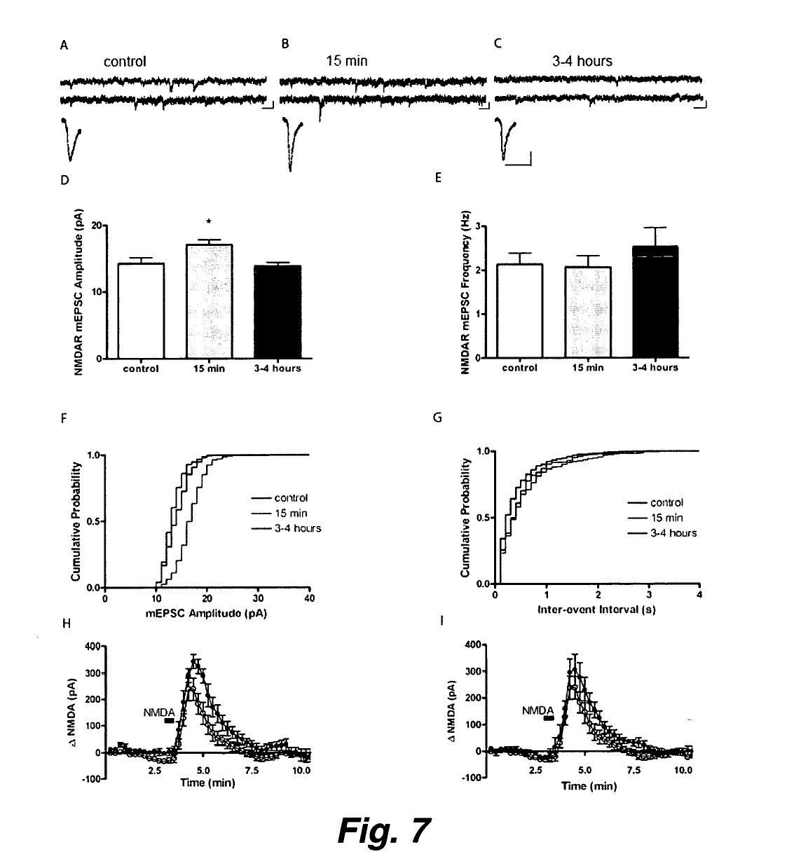

To examine changes in number or function of NMDARs after orexin treatment NMDAR mEPSCs were recorded in buffer without Mg²⁺, and in the presence CNQX (10 μM), glycine (20 μM) and lidocaine (500 μM). Orexin A (100 nM) caused a significant increase in amplitude in NMDAR mEPSCs 15 min after application (17.1±0.7 pA, n=8, FIG. 7B,D,F) compared to untreated neurons (14.2±0.9 pA, n=8, FIG. 7A,D,F) or at 3-4 hours after orexin A application (13.9±0.5 pA, n=7, FIG. 7C,D,F). These results show that the frequency of NMDAR mEPSCs was not significantly altered by orexin A treatment (control: 2.1±0.3, 15 min: 2.1±0.3, 3-4 hours: 2.5±0.4, p>0.05, FIG. 7E,G).

As a final test to determine at what time point after orexin treatment NMDARs were altered, NMDA (50 μM, 30 sec) was bath-applied, and the change in holding current was recorded. There was a significant increase in NMDA-induced current 15 min after orexin A (100 nM) application (orexin A: 344±27 pA, n=7; control: 245±43, n=9, p<0.05, FIG. 7H), but not after 3-4 hours (orexin A: 305±59, n=6; p>0.05, FIG. 7I). Taken together, these results indicate that the number or function or both of AMPARs, is altered 3-4 hours after orexin A exposure, suggesting that orexin A may cause a late-phase long-term potentiation.

Orexin Signaling in the Ventral Tegmental Area is Required for Behavioral Sensitization to Cocaine.

Because orexin A potentiated NMDAR-mediated synaptic responses in dopamine neurons of the VTA, and synaptic plasticity at excitatory synapses after chronic cocaine treatment was blocked with co-injections of the OXR1 antagonist, it was possible that orexin receptor activation contributed to the development of behavioral sensitization to cocaine.

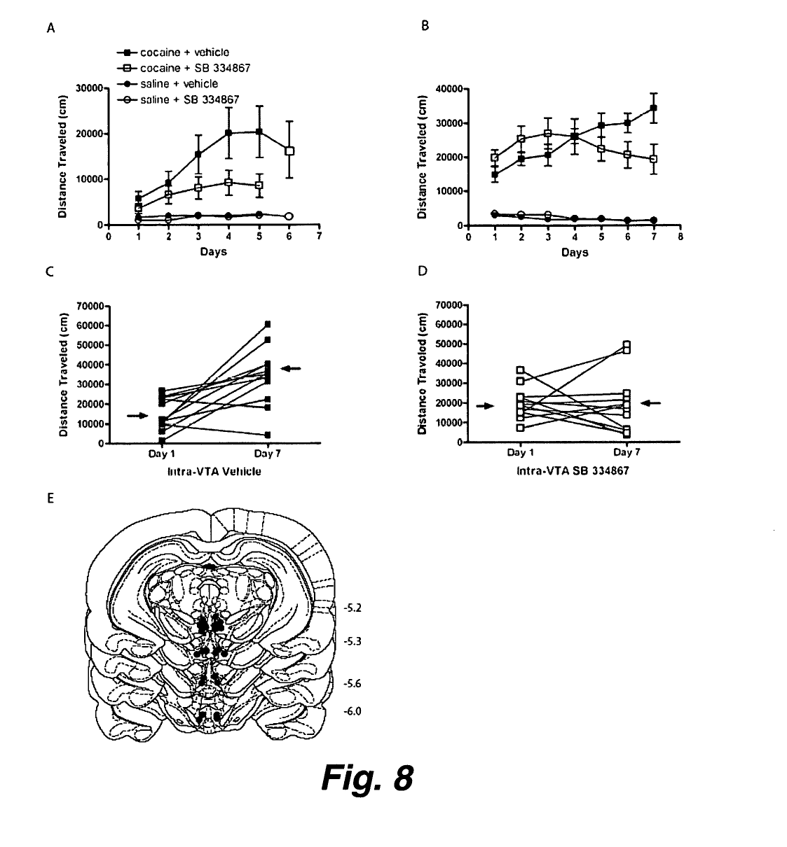

To determine if orexin signaling plays a role in behavioral sensitization to cocaine, either the OXR1 antagonist SB 334867 (10 mg/kg, i.p.) or vehicle was injected 15 min prior to injecting cocaine (15 mg/kg, i.p) or saline (0.9%), and then the rats were placed in an open field chamber to measure locomotor activity for 1 hour each day for 5 days. Interestingly, SB 334867 significantly blocked the development of sensitization as the distance traveled on day 5 was 8504±2564 cm compared to 20347±5643 cm in cocaine-only-treated rats (FIG. 8A, n=13, p<0.05). There was no significant difference in locomotor activity of cocaine-treated rats on day 1 between SB334867 (3579±1079 cm, n=11) and vehicle- (5692±1579 cm, n=13) administered rats (p>0.05). Further, SB 334867 did not reduce baseline locomotor activity (FIG. 8A, n=13, p>0.05).

The foregoing studies provided evidence that orexin A signaling is required for acquisition of cocaine sensitization. However, these experiments had not answered the question of whether orexin A contributes to the expression of cocaine sensitization. Therefore, to determine if expression of cocaine sensitization requires release of orexin acting at the OXR1 receptor, the OXR1 antagonist SB 334867 (10 mg/kg) was injected with cocaine (15 mg/kg) on day 6 into either cocaine-sensitized or saline-treated rats. Locomotor activity on day 6 was not significantly reduced by SB 334867 (FIG. 8A, n=7 in each group, p>0.05), indicating that while orexin signaling is required for the development of cocaine sensitization it is not required for its expression.

The in vitro experiments indicated that VTA was an important site of action for orexin-induced plasticity. Thus, to determine if OXR1 antagonist blockade of locomotor sensitization to cocaine occurred through a direct action in the VTA, the cocaine sensitization experiments were repeated using a 7-day sensitization paradigm, but the OXR1 antagonist was microinjected (6 μg/hemisphere) directly into the VTA prior to cocaine injection (15 mg/kg). FIG. 8B shows that daily intra-VTA injection of the OXR1 antagonist abolished the development of locomotor sensitization to cocaine (FIG. 8B; p>0.05 in SB 334867-treated group, comparing day 7 and day 1 locomotor activity). Control animals injected with vehicle solution showed robust sensitization (FIG. 8B; p<0.01, locomotor activity was significantly higher on days 4-7 relative to day 1). Mean locomotor distance was 34162±4477 cm on day 7 in control animals (FIG. 8C), compared with 19274±4559 cm in OXR1 antagonist-treated animals (FIG. 8D), the latter value not differing significantly from the locomotor distance traveled on day 1. Similar to systemic injections (FIG. 8A), a single intra-VTA injection of the OXR1 antagonist did not affect expression of pre-existing cocaine sensitization in this control group (p>0.05; data not shown). Taken together, these results indicate that OXR1 activation at dopaminergic neurons in the VTA is required for the development of behavioral sensitization as well as for the induction of synaptic plasticity at excitatory synapses associated with sensitization.

Discussion

The data presented here establish a novel function for orexin signaling as a critical substrate for plasticity of synaptic inputs to dopamine neurons of the VTA. Four observations support this conclusion. First, in vitro application of orexin A induces potentiation of NMDAR responses in VTA dopamine neurons. Second, the OXR1 receptor antagonist, SB 334867, blocks induction of cocaine-induced potentiation of excitatory inputs onto VTA neurons. Third, orexin A causes late-phase increases in AMPAR-mediated synaptic transmission. Fourth, microinjection of SB 334867 directly into the VTA blocks the development of cocaine-induced locomotor sensitization. These data provide evidence that orexin signaling pathways play an important role in drug-induced neural plasticity contributing to cocaine addiction.

Orexin A Enhances Synaptic Strength in VTA Dopamine Neurons.

Orexin A potentiated NMDAR EPSCs in dopamine neurons via activation of OXR1 receptors and stimulation of PKC/PLC signal transduction pathways. These results are consistent with orexin A-mediated PLC and PKC activation of Ca²⁺ signaling, observed in isolated VTA neurons (Uramura et al., 2001) through OXR1, that are coupled to Gq-type Ga-proteins (Zhu et al., 2003). The orexin A-mediated synaptic plasticity resulted from the translocation of NMDARs to the synapse. Because there was an increase in NMDAR-evoked EPSCs after orexin treatment compared to controls after blocking all synaptic and extrasynaptic NMDARs, it appears that the orexin-mediated potentiation of NMDARs is mediated, at least in part, by movement of NMDARs from intracellular compartments to the synapse. Movement of NMDARs to synaptic sites could provide a mechanism for rapidly enhancing synaptic strength (Tovar and Westbrook, 2002). This is consistent with orexin A potentiation of synaptic strength by movement of NMDARs into the synapse with a time course on the order of minutes.

Experiments with orexin A application in the presence of NMDAR subunit inhibitors suggests that NR2A-containing NMDARs play a significant role in enhancing NMDAR-mediated responses. In rats aged P21 or older, mRNA and protein for NR2A, NR2B, and to a lesser extent NR2C and NR2D, has been observed in midbrain (Monyer et al., 1994; Dunah et al., 1996), however, NR2A was not observed in the rat substantia nigra (Jones and Gibb, 2005; Standaert et al., 1994). NMDAR subunit composition in rat VTA has not been reported. VTA punches have been found to contain protein for NR2A, NR2B, and NR2C (Schilstrom and Bonci, 2002). NR2D could not be detected due to the lack of selectivity of the antibody. The selectivity of the NR2A/C antagonist has been questioned (Berberich et al., 2005; Weitlauf et al., 2005) because pre-application of 0.4 μM, NVP-AAM077 has been demonstrated to inhibit NR2B-containing NMDARs (Weitlauf et al., 2005). However, three lines of evidence suggest that orexin A can recruit NMDAR subtypes. First, NVP-AAM077 blocked NMDAR potentiation post-orexin A application. Secondly, Zn²⁺, a selective inhibitor of NR2A-containing NMDARs at nanomolar concentrations, blocked orexin-mediated potentiation of NMDARs. Finally, the NR2B subunit antagonist, ifenprodil, exerted no significant effect on orexin A-mediated potentiation of NMDAR EPSCs. The residual orexin-mediated NMDAR potentiation observed in the presence of NVP-AAM077 or Zn²⁺ was blocked by co-application of ifenprodil, NVP-AAM077, and the NR2C/D-preferring antagonist, PPDA. These data suggest that although orexin A primarily potentiates NR2A-containing NMDAR receptors, it can also potentiate other NR2 subunits to a lesser extent. Thus, orexin A appears to be enhancing synaptic plasticity in the VTA primarily by promoting increased NR2A-containing NMDARs at the synapse.

These results indicate that acute orexin A application produces a PLC/PKC-dependent potentiation of NMDAR-mediated responses, while AMPAR-mediated responses are unaffected. Further, in vivo exposure to cocaine produces a delayed potentiation of AMPARs but not NMDARs (Ungless et al., 2001; Borgland et al., 2004; present study), which is blocked by the OXR1 antagonist, SB 334867. Therefore, it is possible that the orexin-mediated potentiation of NMDARs facilitates the induction of synaptic plasticity, which involves an increase in AMPAR-mediated synaptic transmission. Although there was no change in AMPAR-evoked EPSCs, mEPSCs or AMPAR current changes at 15 min after orexin application, by 3-4 hours a robust increase in AMPAR-mediated synaptic transmission had developed. This late-phase increase in post-synaptic AMPAR-mediated synaptic transmission was blocked when the NMDAR antagonist APV was co-administered with orexin A. The increase in AMPAR mEPSC frequency was not blocked by APV, suggesting that an NMDAR-independent delayed presynaptic effect may also occur. Consistent with these results, orexin A has previously been shown to enhance long-term potentiation when applied directly to the dentate gyrus in anesthetized rats (Wayner et al., 2004). Increased synaptic efficacy may be due to insertion of new AMPARs into the synaptic membrane (Malinow et al., 2000; Lu et al., 2001) or other structural or functional modifications at existing AMPAR channels (Benke et al., 1998; Derkach et al., 1999). Additionally, late-phase long-term potentiation occurring 3-5 hours after stimulation is dependent on post-synaptic protein synthesis (Nayak et al., 1998; Grosshans et al., 2001). Therefore, the delayed increase in AMPAR-mediated synaptic transmission after orexin A application may represent late-phase long-term potentiation.

Orexin may Play a Critical Role in Addiction.

The present study demonstrates that orexin A produces long-lasting plasticity at excitatory synapses of dopamine neurons in the VTA. Synaptic plasticity in the VTA has been suggested to play an important role in the behavioral consequences of in vivo exposure to drugs of abuse (Robinson and Berridge, 1993; Overton and Clark, 1997; Ungless et al., 2001; Saal et al., 2003). From a functional perspective, NMDARs perform two major roles in VTA dopamine neurons: they are necessary for the induction of in vitro long-term potentiation (Bonci and Malenka, 1999; Overton et al., 1999) and they promote burst firing of dopamine neurons (Johnson et al., 1992; Overton and Clark, 1992; Komendantov et al., 2004). Previous studies have suggested that burst firing of VTA dopamine neurons encodes the occurrence of salient stimuli (Schultz, 2002). As a consequence, burst firing increases extracellular dopamine in the projection areas more efficiently than when dopamine neurons fire regularly-spaced trains of action potentials (Gonon, 1988; Komendantov et al., 2004). Although dopamine neurons generally fire in a pacemaker-like fashion in vitro, burst firing patterns, similar to what has been observed in vivo, can be reproduced by bath-application of NMDA (Komendantov et al., 2004). Thus, an increase in synaptic efficacy, including the synaptic potentiation observed in this study, may contribute to the increase in dopamine cell firing rate. This change could enhance reinforcement, as it has been proposed that dopamine neuronal firing represents a teaching signal (Garris et al., 1999; Schultz, 2002). Accordingly, orexin A has been demonstrated to increase firing rate and in some cases cause burst firing of VTA dopamine neurons in rat brain slices (Korotkova et al., 2003), although it remains to be determined whether this firing is dependent on potentiation of glutamatergic synapses. Orexin potentiation of NMDAR currents in dopamine neurons may represent a crucial process underlying stimulation of locomotor activity to achieve goal-directed behavior. For example, fasted animals increase wakefulness and locomotor activity via activation of orexin neurons (Yamanaka et al., 2003), and this locomotor activation may be dopamine-dependent (Wisor et al., 2001). The present work has demonstrated that orexin A signaling is required for the acquisition of cocaine sensitization, a paradigm for craving, suggesting that blocking orexin signaling in the VTA, may perturb cocaine's motivational significance and reduce the rat's drive for cocaine seeking.

Animals with lesions in the LH exhibit hypophagia and somnolence (Levitt and Teitelbaum, 1975; Bemardis and Bellinger, 1996), suggesting important roles for the LH in the control of motivated behavior. The LH has also been implicated in motivation associated with reward (Wise 1996; Di Leone et al., 2003). Direct electrical stimulation of the LH is so intensely rewarding to rats that they will starve themselves when limited access to such stimulation is given concurrently with limited access to food (Routtenberg and Lindy, 1965). Furthermore, drugs of abuse potentiate the rewarding effects of LH self-stimulation (Wise 1996). Recent studies have shown that some of these LH functions may require orexin neurons, as orexin knock-out mice show reduced morphine dependence (Georgescu et al., 2003). Additionally, activation of orexin neurons is strongly linked to preferences for cues associated with drug and food reward, as both activation of orexin neurons and intra-VTA administration of orexin A leads to a reinstatement of an extinguished conditioned place preference to morphine (Harris et al., 2005). Therefore, it is a strong possibility that orexin A-induced synaptic plasticity in the VTA is mediating reward-seeking behaviors. The findings presented here establish a potential mechanism for the novel role of orexin signaling in plasticity related to addiction. These results demonstrate that orexin A induces synaptic plasticity in dopaminergic VTA neurons; moreover, this form of synaptic plasticity is likely an important substrate of behaviors relevant to addiction, as we show that activation of OXR1 receptors in the VTA is necessary for the development of cocaine-mediated behavioral sensitization. Thus, orexin receptors provide novel pharmacotherapeutic targets for motivational disorders such as addiction.

Experimental Procedures

Electrophysiology

All of the electrophysiological recordings were performed in rats ranging from P21 to P30. Briefly, rats were anesthetized with halothane and sacrificed. Horizontal sections of the VTA (230 μM) were prepared with a vibratome (Leica, Nussloch, Germany). Slices were placed in a holding chamber and allowed to recover for at least 1 hour before being placed in the recording chamber and superfused with bicarbonate-buffered solution (ACSF) saturated with 95% O₂/5% CO₂ and containing (in mM): 119 NaCl, 1.6 KCl, 1.0 NaH₂PO₄, 1.3 MgCl₂, 2.5 CaCl₂, 26.2 NaHCO₃ and 11 glucose (at 32-34° C.). Picrotoxin (100 μM) was added to block GABAA receptor-mediated inhibitory postsynaptic currents. Cells were visualized using infrared differential interference contrast video microscopy. Whole-cell voltage clamp recordings were made using an Axopatch 1D amplifier (Axon Instruments, Union City, Calif.). Electrodes (2.8-4.0 MΩ) contained (in mM): 120 Cesium Methansulfonate, 20 HEPES, 0.4 EGTA, 2.8 NaCl, 5 TEA-Cl, 2.5 MgATP, and 0.25 NaGTP, pH 7.2-7.3 (270-285 mOsm). Series resistance (10-40 MΩ) and input resistance were monitored on-line with a 4-mV depolarizing step (50 ms) given just after every afferent stimulus. Dopaminergic VTA neurons were identified by the presence of a large Iₕ current (Lacey et al., 1990; Johnson and North, 1992) and, in some cases, tyrosine hydroxylase labeling. A bipolar stimulating electrode was placed 100-300 μm rostral to the recording electrode and was used to stimulate excitatory afferents at 0.1 Hz. Neurons were voltage-clamped at −70 mV and +40 mV to record AMPAR- and NMDAR-mediated EPSCs. EPSCs were filtered at 2 kHz, digitized at 5-10 kHz and collected on-line using Igor Pro software (Wavemetrics, Lake Oswego, Oreg.). NMDAR or AMPAR traces were constructed by averaging 12 EPSCs (120 sec) elicited at +40 mV or at −70 mV. NMDAR amplitude was measured 20 ms after the stimulus artifact when the EPSC is primarily NMDAR-mediated.

To calculate the AMPAR/NMDAR ratio, an average of 12 EPSCs at +40 mV was computed before and after application of the NMDAR blocker, AP5 (50 μM) for 5 min. NMDAR responses were calculated by subtracting the average response in the presence of AP5 (AMPAR only) from that seen in its absence; the peak of the AMPAR EPSC was divided by the peak of the NMDAR EPSC to yield an AMPAR/NMDAR ratio.

AMPAR mEPSCs were recorded in cells voltage-clamped at −70 mV in lidocaine (500 μM), APV (50 μM) and sucrose (100 mM). NMDAR mEPSCs were recorded at −40 mV in Mg2+-free external solution with lidocaine (500 μM), glycine (20 μM), and CNQX (10 μM). mEPSCs were collected using Clampex (Axon Instruments) and analyzed using Mini Analysis Program (Synaptosoft). Detection criteria were set at >10 pA, <1 ms rise-time, and <3 ms decay-time for AMPAR mEPSCs and >10 pA, <1 ms rise-time, and <10 ms decay-time for NMDAR mEPSCs. mEPSC traces were recorded while sampling every 10 μs, the images were filtered at 5 kHz. Averaged mEPSCs were constructed using Mini Analysis Program (Synaptosoft).

Treatment Regimen and Locomotor Activity

Male Sprague-Dawley Rats (P21-P30; Charles River, N.C.) were singly housed with food and water available ad libitum. A 12 hr light/dark cycle was used, with the lights on at 7:00 A.M. All cocaine injections and behavioral testing were performed during the light cycle for consecutive days. The four treatment groups consisted of rats given 5 daily i.p. injections of SB 334867 (10 mg/kg) 15 min preceding i.p. injection of cocaine (15 mg/kg) or saline (0.9% NaCl), or i.p. injections of vehicle (10% DMSO and 25% β-hydroxy cyclodextrin in saline) 15 min preceding i.p. injection of cocaine or saline. All animals were habituated to the photocell boxes (Med Associates, St. Albans, Vt.) for 2 hours prior to the start of the experiment. All animals were further habituated to the photocell boxes for 30 min prior to 60 min testing session. Locomotor activity was measured in 17″×17″ chambers lined with three 16-beam I/R arrays. A 50 ms scanning rate was used for measurement of beams broken. Distance traveled was measured using Open Field Activity Software (Med Associates, Inc) and analyzed for estimates of locomotion based on the movement of a given distance and resting delay (movement in a given period of time).

Intra-VTA Injections and Locomotor Activity

Male Sprague-Dawley rats (250 grams) were implanted with bilateral cannulae directed at the VTA (AP, −5.2; ML, ±0.5, DV −8.0). Following recovery from surgery, animals were tested in a cocaine sensitization assay similar to that used for systemic administration of the OXR1 antagonist, but with two changes: a 7-day sensitization paradigm was used, and the OXR1 antagonist SB 334867 was microinjected directly into the VTA (6 μg/hemisphere in 300 nL solution composed of 10% DMSO in water). All other methods for habituation and data collection were identical to that described above. Rats were deeply anesthetized with pentobarbital, and perfused with a 10% formaldehyde solution. Brains were cryoprotected in 25% sucrose, 40 μm coronal sections were cut on a freezing microtome, and sections were mounted, dried and coverslipped. Injection sites were located under a light microscope and recorded on atlas figures adapted from Paxinos and Watson (1997).

Data Analysis

All values are expressed as mean±s.e.m. Statistical significance was assessed using two-tailed Student's t-tests or a one-way ANOVA for multiple group comparisons. A Bonferroni post-hoc test following an ANOVA was used to test significant differences between multiple groups.

### Example 2

### CRF and Orexin Antagonists Reduce Ethanol Consumption and Cocaine Reinstatement in Rats

Methods

Stereotaxic Implantation of Cannulae

Agents in these in vivo studies were administered to either the lateral ventricle (icv) or ventral tegmental area (VTA) in rats by cannulae, which were implanted as follows. One week after the rats achieved baseline drinking and lever pressing levels in the 2-bottle choice and ethanol self-administration paradigms described below, they were anesthetized with 75 mg/kg pentobarbital sodium and placed in a stereotaxic frame. The head was shaved and scrubbed with an antiseptic solution of betadine/2% H₂O₂, followed by 2.5% topical xylocalne jelly. The skull was exposed and two to three holes were drilled for implantation of sterile miniature skull screws (⅛″, Small Parts, Inc.). The microinjection guide cannula (25 gauge, stainless steel, Plastics One) was then drilled into the skull directly above the VTA or lateral ventricle. The coordinates for the intracerebroventricle guide cannulae were as follows: 0.8 mm, anterior-posterior, 1.5 mm medial-lateral, and −3.5 mm dorso-ventral −3.5 mm. The coordinates for VTA guide cannulae were as follows: 5.6 mm posterior to bregma, 2.2 mm lateral at an angle of 12° toward the midline, and 6.7 mm ventral to the skull surface according to the atlas of Paxinos and Watson (1986). The cannula was anchored to the skull with stainless-steel screws and dental cement. The guide cannula was then stereotaxically lowered below the skull surface to a depth 2.5 mm above the desired injection location and secured with dental acrylic resin. A dummy cannula was then inserted into the guide cannulae. The wound was treated with 2.5% topical xylocalne jelly and bacitracin ointment and closed with 3-0 silk. All subjects were then returned to their home cages with ad libitum access to food and water and allowed to recover for at least 5 days prior to any behavioral manipulation. Infusions of vehicle (either phosphate buffered saline for CRF, CRF (6-33), and orexin or DMSO for the orexin antagonist) or CRF peptide agonists and antagonists or orexin were delivered in a volume of 0.5 μl.

Voluntary Ethanol Consumption Using Limited-Access Two-Bottle Choice

This procedure measures voluntary ethanol consumption in Long Evans rats, a species that has been shown to have a high preference for ethanol (Khanna, et al., 1990). These experiments test the effects of the CRF system on voluntary ethanol consumption using a limited-access paradigm. Rats were housed individually in ventilated Plexiglas cages equipped with two bottle grommets on one end of each cage. Fluids were presented daily in 100-ml graduated glass cylinders with stainless steel drinking spouts inserted through the front of the cage. Prior to training, rats were given at least one week to acclimatize to the individual housing conditions and handling. During this period, water was the only fluid available. Rats were then given concurrent access to a solution containing 10% (v/v) ethanol+10% (w/v) sucrose and a separate water bottle. Over the next 12 days, the sucrose concentration was gradually decreased (i.e. 5, 2, and finally 0%). Measurements were taken to the nearest gram. Animal weights were measured daily in order to calculate the gram per kilogram intake. Ethanol preference (%) was calculated as the grams of ethanol consumed divided by the total fluid consumption (grams of ethanol+grams of water). The data was corrected for evaporation and spillage by subtracting the mean fluid loss measured in four drinking tubes placed on empty cages. The position of the tubes (left/right) was alternated to control for side preferences.

When drinking was stable at 10% ethanol (usually 2-3 weeks following removal of the sucrose), guide cannulae are surgically implanted into the lateral ventricle as described above. For the week following the surgery, the rats had continuous access to 10% ethanol and water. On the second week post-surgery, the rats were shifted to a 2-hour limited access procedure with ethanol available after the onset of the dark cycle (Smith et al., 1999). These two-hour limited access sessions were conducted four nights per week (i.e., Mon.-Thurs.) for two weeks. Drug administrations were begun after the rats had reached stable drinking levels of the 10% v/v ethanol solution (2-3 weeks following surgery) within the two-hour access period.

Operant Cocaine or Food Self-Administation

Rats were cannulated with an intra-venous cannula to their jugular vein. After one week to 10 days of recovery, the animals were trained to lever press for cocaine (0.5 mg/infusion, 2 second infusion) in Coulburn self-administration boxes. A tone and light above the active lever signalled during a press for cocaine and then a 20 second timeout followed the active lever press. Both active and inactive lever presses were recorded.

After 3-4 weeks of self-administration of cocaine (0.5 mg/infusion) ramping from fixed ratio of 1 lever press (FR1) to receive a reward to a stable fixed ratio of 5 lever presses to receive a reward (FR5) schedule, animals lever pressed for cocaine on a progressive ratio schedule for 4 days. On the second and third day, each animal received a vehicle injection (i.p.), and on the fourth day the animals received an injection (i.p.) of the OXR1 antagonist: SB334867, 10 mg/kg. As a control, orexin modulation of food self-administration was examined under the same paradigm. For food self-administration, animals were trained to an FR5 schedule for food pellets. Both cocaine and food trained animals received 20 g food per day, an amount that is not restrictive, but does not increase the mass of the rats.

Results and Discussion

The CRF Antagonist CRF (6-33) Inhibits the Consumption of Ethanol in the Development of Alcohol Dependence and Relapse

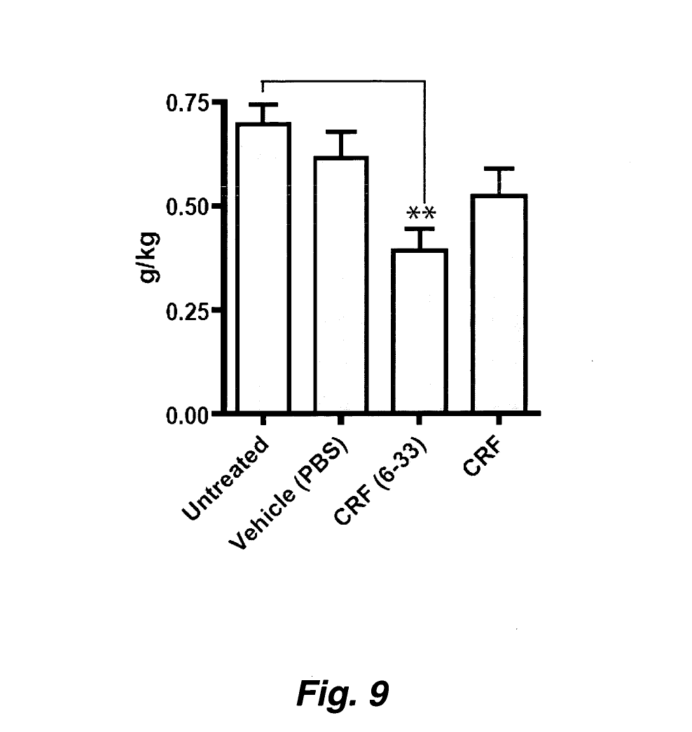

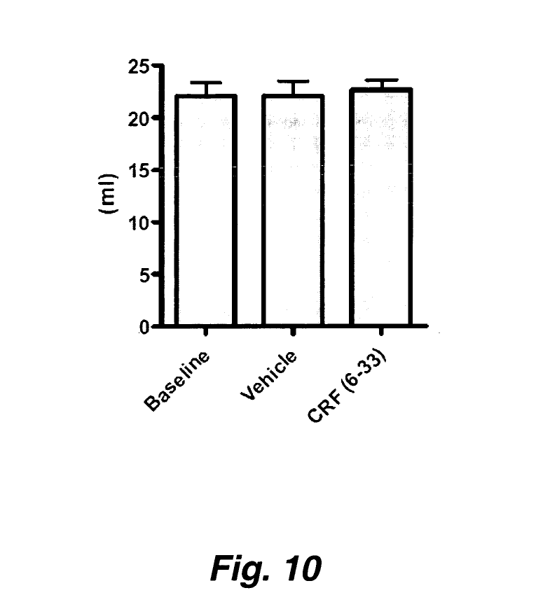

The effects of CRF (5 μg) and the CRF inhibitor CRF (6-33) (5 μg) on limited access (30 min) 10% ethanol drinking were measured using a limited-access two-bottle choice procedure. ICV CRF (6-33) peptide significantly reduced 10% ethanol consumption compared to CRF alone (FIG. 9, n=14) with no effect on water consumption (FIG. 10, n=14). These data indicate that inhibition of the CRF pathway can modulate alcohol-mediated behaviors in rats. These results are consistent with the recent finding that CRF antagonists injected into the VTA have been shown to reduce footshock stress-induced relapse to cocaine-seeking behaviors (Wang B, Shaham Y, Zitzman D, Azari S, Wise R A, You Z B. Cocaine experience establishes control of midbrain glutamate and dopamine by corticotropin-releasing factor: a role in stress-induced relapse to drug seeking. J. Neurosci. 2005 Jun. 1; 25(22):5389-96).

The OXR1 Antagonist SB 334867 Reduces Cocaine Reinforcement

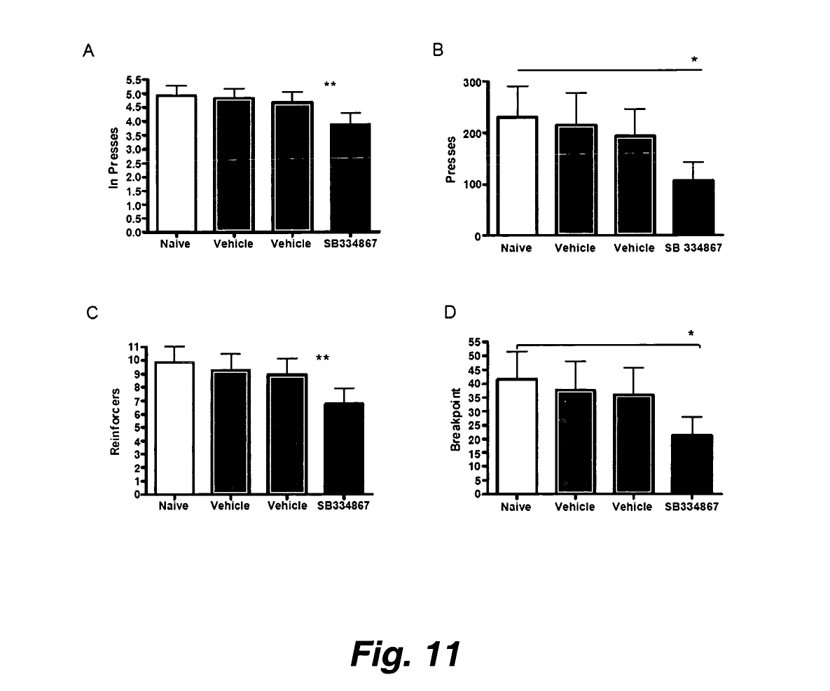

Rats were trained to lever press for cocaine (0.5 mg/I.V. infusion on an fixed ratio (one lever press to receive the cocaine [the reinforcer]; FR1) or fixed ratio of 5 lever presses (FR5) schedule of reinforcement and subsequently a progressive ratio schedule, where the number of lever presses progressively increased. Rats were given vehicle on the 2^{nd} and 3^{rd} day of progressive ratio testing and then SB 334867 (10 mg/kg, i.p.) on the 4^{th} day. FIG. 11A shows that the natural log of active lever presses for cocaine was reduced after SB334867 administration (p<0.01, n=12). FIG. 11B shows that raw active lever presses was reduced after SB334867 administration (p<0.05, n=12). FIG. 11C shows that the total number of cocaine infusions was decreased after SB334867 administration (p<0.01, n=12). FIG. 11D shows that the breakpoint was reduced after SB334867 administration (p<0.05, n=12).

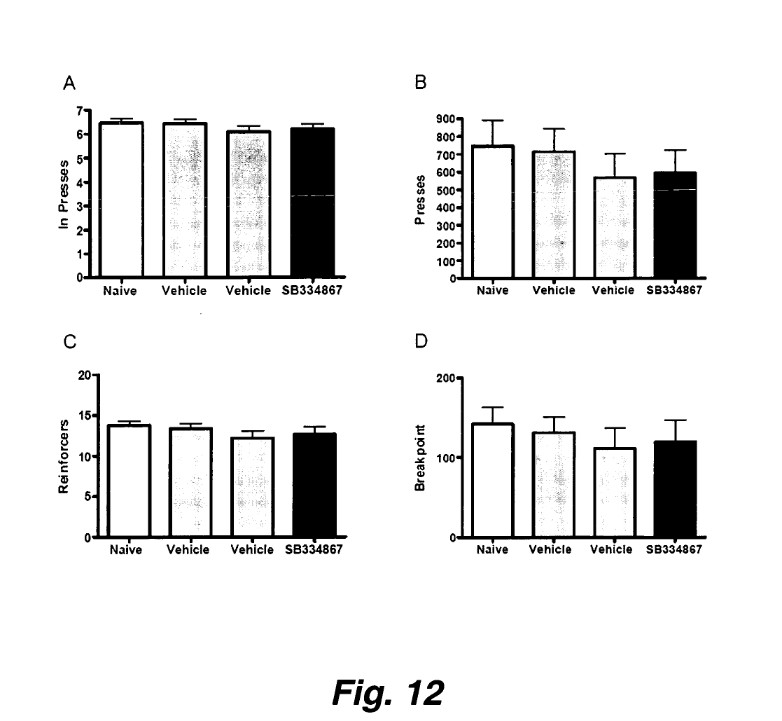

This effect of the OXR1 antagonist was not observed with respect to reinforcement for food. Rats were trained to lever press for food on an FR1 or FR3 schedule, and subsequently a progressive ratio schedule. Rats were given vehicle on the 2^{nd} and 3^{rd} day of progressive ratio testing and then SB 334867 (10 mg/kg, i.p.) on the 4^{th} day. FIG. 12A indicates that the natural log of active lever presses for food (p<0.05, n=10). FIG. 12B shows that raw active lever presses (p<0.05, n=10). FIG. 12C shows that the total number of food pellets received was not altered after SB334867 administration (p<0.05, n=10). FIG. 12D shows that the breakpoint was unaltered after SB334867 administration (p<0.05, n=10).

### Example 3

### Orexin A Potentiates the Effect of CRF on NMDAR-Mediated EPSCs

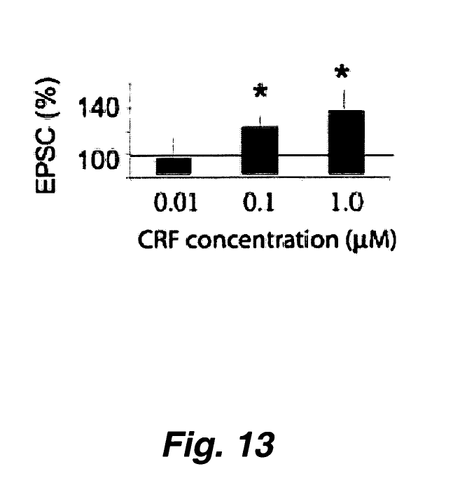

CRF increased NMDAR eEPSCs in a concentration dependent manner in mice. (FIG. 13) There was no potentiation of NMDARs after application of 10 nM CRF. Ungless et al., 2003 Neuron 39: 401-7.

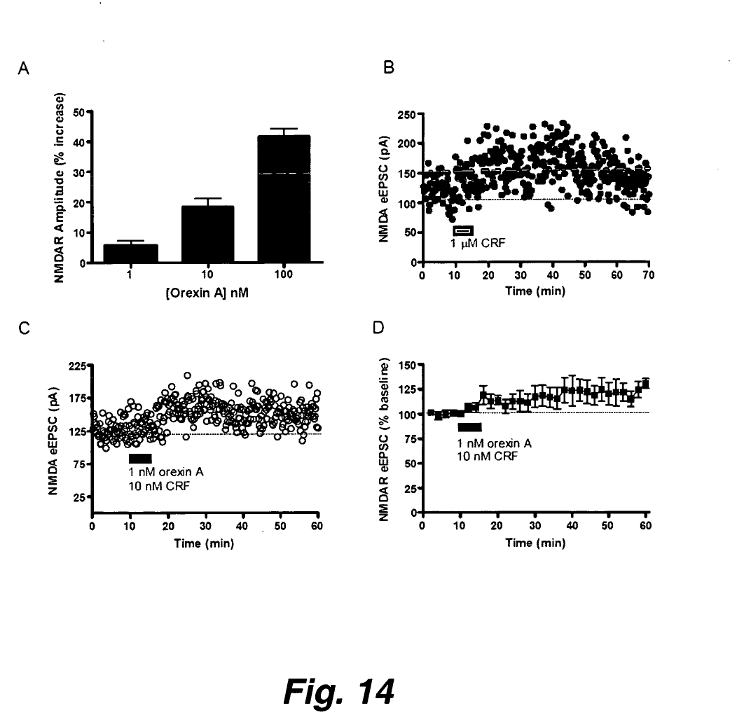

The effects of Orexin A and CRF on NMDAR-mediated EPSCs in the rat VTA were measured as described in Example 1. FIG. 14A indicates that Orexin A at 1 nM potentiates NMDARs 5.7±1.6% (n=6). FIG. 14B shows an example of a trace trace of a 5 min application of CRF (1 μM) on NMDAR eEPSCs in rats. FIG. 14C shows an example of a trace of NMDAR eEPSCs after a 5 min co-application of orexin A (1 nM) with CRF (10 nM). FIG. 14D shows that the application of orexin A (1 nM) with CRF (10 nM) significantly potentiates NMDAR eEPSCs to a maximum of 123±12% (p<0.05, n=7).

### REFERENCES

Baldo, B. A., Daniel, R. A, Berridge, C. W., and Kelley, A. E. (2003). Overlapping distributions of orexin/hypocretin- and dopamine-β-hydroxylase immunoreactivity fibers in rat brain regions mediating arousal, motivation, and stress. J. Comp. Neurol. 464, 220-237.
Benke, T. A., Luthi, A., Isaac, J. T., and Collingridge, G. L. (1998). Modulation of AMPA receptor unitary conductions by synaptic activity. Nature 393, 793-797.
Berberich, S., Punnakkal, P., Jensen, V., Pawlak, V., Seeburg, P. H., Hvalby, O., and Kohr, G. (2005). Lack of NMDA receptor subtype selectivity for hippocampal long-term potentiation. J. Neurosci. 25, 6907-6910.
Bernardis, L. L., and Bellinger, L. L. (1996). The lateral hypothalamic area revisited: ingestive behavior. Neurosci. Biobehav. Rev. 20, 189-287.
Bonci, A., and Malenka, R. C. (1999). Properties and plasticity of excitatory synapses on dopaminergic and GABAergic cells in the ventral tegmental area. J. Neurosci. 19, 3723-3730.
Borgland, S. L., Malenka, R., and Bonci, A. (2004). Acute and chronic cocaine-induced potentiation of synaptic strength in the VTA: electrophysiological and behavioral correlates in individual rats. J. Neurosci. 24, 7482-7490.
Cameron, D. L., Wessendorf, M. W., and Williams, J. T. (1997). A subset of ventral tegmental area neurons is inhibited by dopamine, 5-hydroxytryptamine and opioids. Neuroscience 77, 155-166.
Carlezon, W. A., and Nestler, E. J. (2002). Elevated levels of GluR1 in the midbrain: a trigger for sensitization to drugs of abuse? Trends Neurosci. 25, 610-615.
Cull-Candy, S., Brickley, S., and Farrant, M. (2001). NMDA receptor subunits: diversity, development and disease. Curr. Opin. Neurobiol. 11, 327-335.
Derkach, V., Barria, A., and Soderling, T. R. (1999). Ca2+/calmodulin-kinase II enhances channel conductance of alpha-amino-3-hydroxy-5-methyl-4-isoxazolepropionate type glutamate receptors. Proc. Natl. Acad. Sci. USA 96, 3269-3274.
DeLecea, L., Kilduff, T. S., Peyron, C., Gao, X-B., Foye, P. E., Danielson, P. E., Fukuhara, C., Battenberg, E. L., Gautvik, V. T., Bartless, F. S., Frankel, W. N., van den Pol, A. N., Bloom, F. E., Gautvik, K. M., and Sutcliffe, J. G. (1998). The hypocretins: hypothalamus-specific peptides with neuroexcitatory activity. Proc. Natl. Acad. Sci. USA 95, 322-327.
DiLeone, R. J., Georgescu, D., and Nestler, E. J. (2003). Lateral hypothalamic neuropeptides in reward and drug addiction. Life Sci. 73, 759-768.
Dunah, A. W., Yasuda, R. P., Wang, Y-H, Luo, J., Davila-Garcia, M. I., Gbadegesin, M., Vicini, S., and Wolfe, B. B. (1996). Regional and Ontogenic Expression of the NMDA receptor subunit NR2D protein in rat using a subunit-specific antibody. J. Neurochem. 67, 2335-2345.
Erreger K., Dravid, S. M., Banke, T. G., Wyllie, D. J., and Traynelis, S. F. (2005). Subunit-specific gating controls rat NR1/NR2A and NR1/NR2B NMDA channel kinetics and synaptic signaling profiles. J. Physiol. (Lond). 563, 345-358.
Fadel, J., and Deutch, A. Y. (2002). Anatomical substrates of orexin-dopamine interactions: lateral hypothalamic projections to the ventral tegmental area. Neuroscience 111, 379-387.
Feng, B., Tse, H. W., Skifter, D. A., Morley, R., Jane, D. E., and Monaghan, D. T. (2004). Structure-activity analysis of a novel NR2C/NR2D-preferring NMDA receptor antagonist: 1-(phenantrene-2carbonyl) piperazine-2,3-dicarboxylic acid. Br. J. Pharmacol. 141, 508-516.
Fitzgerald, L. W., Ortiz, J., Hamedani, A. G., and Nestler, E. J. (1996). Drugs of abuse and stress increase the expression of GluR1 and NMDAR1 glutamate receptor subunits in the rat ventral tegmental area: common adaptations among cross-sensitizing agents. J. Neurosci. 16, 274-282.
Fong, D K., Rao, A., Crump, F. T., and Craig, A. M. (2002). Rapid synaptic modeling by protein kinase C: reciprocal translocation of NMDA receptors and calcium/calmodulin dependent kinase II. J. Neurosci. 22, 2153-2164.
Garris, P. A., Kilpatrick, M., Bunin, M. A., Michael, D., Walker, Q. D., and Wightman, R. M. (1999). Dissociation of dopamine release in the nucleus accumbens from intracranial self-stimulation. Nature 398, 67-69.
Georgescu, D., Zachariou, V., Barrot, M., Mieda, M., Willie, J. T., Eisch, A. J., Yanagisawa, M., Nestler, E. J., and DiLeone, R. J. (2003). Involvement of lateral hypothalamic peptide orexin in morphine dependence and withdrawal. J. Neurosci. 23, 3106-3111.
Gonon, F. G. (1988). Non-linear relationship between impulse flow and dopamine released by rat midbrain dopaminergic neurons as studied by in vivo electrochemistry. Neuroscience 24, 19-28.
Grosshans, D. R., Clayton, D. A., Coultrap, S. J., and Browning, M. D. (2001). LTP leads to rapid surface expression of NMDA but not AMPA receptors in adult rat CA1. Nat. Neurosci. 5, 27-33.
Harris, G. C., Wimmer, W., and Aston Jones, G. (2005). A role for lateral hypothalamic orexin neurons in reward seeking. Nature 437, 556-559.
Johnson, S. W., and North, R. A. (1992). Two types of neurone in the rat ventral tegmental area and their synaptic inputs. J. Physiol. 450, 455-468.
Johnson, S. W., Seutin, V., and North, R. A. (1992). Burst firing in dopamine neurons induced by N-methyl-D-aspartate: role of electrogenic sodium pump. Science 258, 665-667.
Jones, S., and Gibb, A. J. (2005). Functional NR2B- and NR2D-containing NMDA receptor channels in rat substantia nigra dopamine neurones. J. Physiol. 569, 209-221.
Kauer, J. A. (2004). Learning mechanisms in addiction: synaptic plasticity in the ventral tegmental area as a result of exposure to drugs of abuse. Annu. Rev. Physiol. 66, 447-475.
Kim, J. A., Pollak, K. A., Hjelmstad, G. O., and Fields, H. L. (2004). A single cocaine exposure enhances both opioid reward and aversion through a ventral tegmental area-dependent mechanism. Proc. Natl. Acad. Sci. USA 101, 5664-5669.
Komendantov, A. O., Komendantova, O. G., Johnson, S. W., and Canavier, C. (2004). A modeling study suggests complementary roles for GABAA and NMDA receptors and the SK channel in regulating the firing pattern in midbrain dopamine neurons. J. Neurophysiol. 91, 346-357.
Korotkova, T. M., Sergeeva, O. A., Eriksson, K. S., Haas, H. L., and Brown, R. E. (2003). Excitation of ventral tegmental area dopaminergic and nondopaminergic neurons by orexin/hypocretins. J. Neurosci. 23, 7-11.
Lacey, M. G., Mercuri, N. B., and North, R. A. (1990). Actions of cocaine on rat dopaminergic neurons in vitro. Br. J. Pharmacol. 99, 731-735.
Lan, J-Y., Skeberdis, V. A., Jover, T., Grooms, S. Y., Lin, Y., Araneda, R. C., Zhen, X., Bennett, M. V. L., and Zukin, R. S. (2001). Protein kinase C modulates NMDA receptor trafficking and gating. Nat. Neurosci. 4, 382-390.
Levitt, D. R., and Teitelbaum, P. (1975). Somnolence, akinesia, and sensory activation of motivated behavior in the lateral hypothalamic syndrome. Proc. Natl. Acad. Sci. USA. 72, 2819-2823.
Liu, L., Wong, T. P., Pozza, M. F., Ligenhoehl, K., Wang, Y., Sheng, M., Auberson, Y. P., and Wang, Y. T. (2004). Role of NMDA receptor subtypes in governing the direction of hippocampal synaptic plasticity. Science 304, 1021-1024.
Lu W., Man H., Ju, W., Trimble, W. S., Macdonald, J. S., and Wang, Y. T. (2001). Activation of synaptic NMDA receptors induces membrane insertion of new AMPA receptors and LTP in cultured hippocampal neurons. Neuron 29, 243-254.
Malinow, R., Schulman, H., and Tsien, R. W. (1989). Inhibition of postsynaptic PKC or CaMKII blocks induction but not expression of LTP. Science 245, 862-866.
Malinow, R., Mainen, Z. F., and Hiyashi, Y. (2000). LTP mechanisms: from silence to four lane traffic. Curr. Opin. Neurobiol. 10, 352-357.
Margolis, E. B., Hjelmstad, G. O., Bonci, A., and Fields, H. L. (2003). k-Opioid agonists directly inhibit midbrain dopaminergic neurons. J. Neurosci. 23, 9981-9986.
Monyer, H., Burnashev, N., Laurie, D. J., Sakmann, B., and Seeburg, P. H. (1994). Developmental and regional expression in the rat brain and functional properties of four NMDA receptors. Neuron. 12, 529-540.
Nakamura, T., Uramura, K., Nambu, T., Yada, T., Goto, K., Yanagisawa, M., and Sakurai, T. (2000). Orexin-induced hyperlocomotion and stereotypy are mediated by the dopaminergic system. Brain Res. 873, 181-187.
Nayak, A., Zastrow, D. J., Lickteig, R., Zahniser, N. R., and Browning, M. D. (1998). Maintenance of late-phase LTP is accompanied by PKA-dependent increase in AMPA receptor synthesis. Nature 394, 680-683.
Nestler, E. J. (2001). Molecular basis of long-term plasticity underlying addiction. Nature Rev. 2, 119-128.
Overton, P. G., and Clark, D. (1992). Iontophroetically administered drugs acting at the N-methyl-D-aspartate receptor modulate burst firing in A9 dopamine neurons in the rat. Synapse 10, 131-140.
Overton, P. G., and Clark, D. (1997). Burst firing in midbrain dopaminergic neurons. Brain Res. Brain Res. Rev. 25, 312-334.
Overton, P. G., Richards, C. D., Berry, M. S., and Clark, D. (1999). Long-term potentiation at excitatory amino acid synapses on midbrain dopamine neurons. Neuroreport 10, 221-226.
Paoletti, P., Ascher P., and Neyton, J. (1997). High-affinity zinc inhibition of NMDA NR1-NR2A receptors. J. Neurosci. 17, 5711-5725.
Paxinos, G., and Watson, C. (1997) The rat brain in sterotaxic coordinates. New York: Academic Press.
Robinson, T. E., and Berridge, K. C. (1993). The neural basis of drug craving; an incentive-sensitization theory of addiction. Brain Res. Rev. 18, 247-291.
Rosemund, C., Clements, J. D., and Westbrook, G. L. (1993). Non-uniform probability of glutamate release at a hippocampal synapse. Science 262, 754-757.
Routtenberg, A., and Lindy, J. (1965). Effects of the availability of rewarding septal and hypothalamic stimulation on bar pressing for food under conditions of deprivation. J. Comp. Physiol. Psychol. 65, 158-161.
Saal, D., Dong, Y., Bonci, A., and Malenka, R. C. (2003). Drugs of abuse and stress trigger a common synaptic adaptation in dopamine neurons. Neuron 37, 577-582.
Sakurai, T., Amemiya, A., Ishii, M., Matsuzaki, I., Chemelli, R. M., Tanaka, H., Williams, S. C., Richardson, J. A., Kozlowski, G. P., Wilson, S., Arch, J. R., Buckingham, R. E., Haynes, A. A. C., Carr, S. A., Annan, R. S., McNulty, D. E., Liu, W. S., Terrett, J. A., Elshourbagy, N. A., Bergsma, D. J., and Yanagisawa, M. (1998). Orexin and orexin receptors: a family of hypothalamic neuropeptides and G protein-coupled receptors that regulate feeding behavior. Cell 92, 573-585.
Schilstrom, B., and Bonci, A. (2002). Altered function of NMDA receptors in VTA dopamine neurons in vitro following a brief exposure to cocaine. Proc. Soc. Neurosci. 897.16.
Schultz, W. (2002). Getting formal with dopamine reward. Neuron 36, 241-263.
Scott, D. B., Blanpied, T. A., Swanson, G. T., Zhang, C., and Ehlers, M. D. (2001). An NMDA receptor ER retention signal regulated by phosphorylation and alternative splicing. J. Neurosci. 21, 3063-3072.
Shippenberg, T. S., and Heidbreder, C. (1995). Sensitization to the conditioned rewarding effects of cocaine: pharmacological and temporal characteristics. J. Pharmacol. Exp. Ther. 273, 808-815.
Standaert, D. G., Testa, C. M., Young, A. B, and Penney, J. B. (1994). Organization of N-Methyl-D-Aspartate glutamate receptor gene expression in the basal ganglia of the rat. J. Comp. Neurol. 343, 1-16.
Thomas, M. J., Beurrier, C., Bonci, A. and Malenka, R. C. (2001). Long-term depression in the nucleus accumbens: a neural correlate of behavioural sensitization to cocaine. Nat. Neurosci. 4, 1217-1223.
Tovar, K. R., and Westbrook, G. L. (2002). Mobile NMDA receptors at hippocampal synapses. Neuron 34, 255-264.
Ungless, M. A., Whistler, J. L., Malenka, R. C., and Bonci, A. (2001). A single cocaine exposure in vivo induces long-term potentiation in dopamine neurons. Nature 411, 583-587.
Uramura, K., Funahashi, H., Muroya, S., Shioda, S., Takigawa, M., and Yada, T. (2001). Orexin A activates phospholipase C- and protein Kinase C-mediated Ca²⁺ signaling in dopamine neurons of the ventral tegmental area. NeuroReport 12, 1885-1889.
Vanderschuren, L. J., and Kalivas, P. W. (2000). Alterations in dopaminergic and glutamatergic transmission in the induction and expression of behavioral sensitization: a critical review of preclinical studies. Psychopharmacol. 151, 99-120.
Wayner, M. J., Armstrong, D. L., Phelix, C. F., and Oomura, Y. (2004). Orexin-A (Hypocretin-1) and leptin enhance LTP in the dentate gyrus of rats in vivo. Peptides 25, 991-996.
Weitlauf, C., Honse Y., Auberson, Y., Mishina, M., Lovinger, D., and Winder, D. (2005). Activation of NR2A-containing NMDA receptors is not obligatory for NMDA-receptor dependent LTP. J. Neurosci. 25, 8386-8390.
Wise, R. (1996). Addictive drugs and brain stimulation reward. Annu. Rev. Neurosci. 19, 319-340.
Wisor, J. P., Nishino, S., Sora, I., Uhl, G. H., Mignot, E., and Edgar, D. M. (2001). Dopaminergic role in stimulant-induced wakefulness. J. Neurosci. 21, 1787-1794.
Yamanaka, A., Beuckmann, C. T., Willie, J. T., Hara, J., Tsujino, N., Mieda, M., Tomnaga, M., Yagami, K., Sugiyama, F., Goto, K., Yanagisawa, M., and Sakurai, T. (2003). Hypothalamic orexin neurons regulate arousal according to energy balance in mice. Neuron 38, 701-713.
Zhu, Y., Miwa, Y., Yamanaka, A., Yada, T., Shibahara, M., Abe, Y., Sakurai, T., and Goto, K. (2003). Orexin receptor type-1 couples exclusively to pertussis toxin-insensitive G-proteins, while orexin receptor type-2 couples to both pertussis toxin-sensitive and -insensitive G-proteins. J. Pharmacol. Sci. 92, 259-266.

1. A method of modulating a N-methyl-D-aspartate receptor (NMDAR)-mediated current, said method comprising administering to a mammal, an orexin receptor agonist or antagonist in a concentration sufficient to alter said NMDAR-mediated current.

2. The method of claim 1, wherein said orexin receptor agonist or antagonist is selective for the orexin receptor type 1 (OXR1).

3. The method of claim 1, wherein said method comprises administering an orexin receptor antagonist to downregulate said NMDAR-mediated current.

4. The method of claim 1, wherein said orexin receptor antagonist is selected from the group consisting of tetrahydroisoquinolines, aroyl piperazine derivatives, 1-(2-methylbenzoxazol-6-yl)-3-[1,5]naphthyridin-4-yl urea hydrochloride (SB-334867-A), N-(6,8-difluoro-2-methyl-4-quinolinyl)-N′-[4-(dimethylamino) phenyl]urea (SN-408124), phenyl urea derivatives, and phenyl thiourea derivatives.

5. The method of claim, wherein said method comprises administering an orexin receptor agonist to upregulate said NMDAR-mediated current.

6. The method of claim, wherein said orexin receptor agonist is selected from the group consisting of orexin A, orexin B, and [Ala¹¹,D-Leu¹⁵]-orexin B.

7. The method of claim, wherein said mammal is a mammal not being treated for an eating disorder.

8. The method of claim, wherein said method further comprises administering a CRF receptor agonist or antagonist.

9. A method of mitigating a symptom of substance abuse in a mammal, said method comprising administering to the mammal, an orexin receptor antagonist in a concentration sufficient to reduce or prevent a symptom of substance abuse, wherein said substance of abuse is selected from the group consisting of an opioid, a psychostimulant, a sedative-hypnotic drug, a cannabinoid, an empathogen, a dissociative drug, alcohol, and nicotine.

10. (canceled)

11. A method of mitigating a symptom of substance abuse in a mammal, said method comprising administering to the mammal, an orexin receptor antagonist in a concentration sufficient to reduce or prevent a symptom of substance abuse, wherein said symptom is selected from the group consisting of reward, incentive salience, craving, preference, seeking, and/or intake (self-administration) of said substance of abuse; relapse; and a symptom of withdrawal.

12. A method of mitigating a symptom of substance abuse in a mammal, said method comprising administering to the mammal, an orexin receptor antagonist in a concentration sufficient to reduce or prevent a symptom of substance abuse, wherein said method further comprises administering to said mammal a corticotrophin-releasing factor (CRF) receptor antagonist.

13-17. (canceled)

18. A method of modulating a N-methyl-D-aspartate receptor (NMDAR)-mediated current in a dopaminergic neuron, said method comprising modulating binding between orexin and the orexin receptor type 1 (OXR1).

19. A method of modulating the activity of corticotrophin-releasing factor (CRF) on a dopaminergic neuron, said method comprising modulating binding between orexin and the orexin receptor type 1 (OXR1)

20. A method of modulating a N-methyl-D-aspartate receptor (NMDAR)-mediated current in a mammal, said method comprising administering to said mammal an orexin receptor agonist or antagonist in conjunction with a corticotrophin-releasing factor (CRF) receptor agonist or antagonist.

21-27. (canceled)

28. A composition comprising an orexin receptor agonist or antagonist combined with a CRF receptor agonist or antagonist.

29-33. (canceled)

34. A method of screening for an agent that modulates orexin potentiation of N-methyl-D-aspartate receptor (NMDAR)-mediated currents, said method comprising:
contacting a cell with a test agent; and
detecting the expression or activity of an orexin receptor type 1 (OXR1);
wherein an alteration of expression or activity of an OXR1 receptor as compared to a control indicates that said test agent is an agent that modulates orexin potentiation of NMDAR-mediated currents.

35-49. (canceled)

50. A method of screening for an agent that modulates the activity of orexin on a dopaminergic neuron, said method comprising:
contacting a test agent with an orexin and/or an orexin receptor type 1 (OXR1);
and detecting an increase or decrease in interaction between said orexin and said OXR1 receptor where an increase or decrease in said interaction, as compared to a control, indicates that said test agent modulates the activity of orexin on a dopaminergic neuron.

51-57. (canceled)
